(19) United States
(12) Patent Application Publication
(10) Pub. No.: US 2008/0268044 A1
(43) Pub. Date: Oct. 30, 2008

(54) Novel Process and Formulations

(75) Inventors: John Robert George Appleby, Hertfordshire (GB); Philip Blatcher, Hertfordshire (GB); Ann M. Diederich, Durham, NC (US); Lesley Anne Humphries, Hertfordshire (GB); Jiri Kasparec, Collegeville, PA (US); As'ad Abu Khalil, Essex (GB); Richard S. Lloyd, Hertfordshire (GB); Paul G. Spoors, King of Prussia, PA (US); John J. Taggart, King of Prussia, PA (US); Lois E. Vernon, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 12/093,191

(22) PCT Filed: Nov. 15, 2006
(86) PCT No.: PCT/US06/60898

§ 371 Date: May 9, 2008

Related U.S. Application Data
(60) Provisional application No. 60/736,679, filed on Nov. 15, 2005.

(51) Int. Cl. C07D 487/04; A61K 31/519; A61K 9/20; A61P 1/00; A61P 9/00; A61P 11/00

(52) U.S. Cl. 424/464; 514/264.1; 544/279

(57) ABSTRACT

The present invention provides for a novel process of making 6-carboxylic acid derivatives of pyrido[2,3-d]pyrimidin-7-one's, as well as a novel process for making 8-(2,6-difluorophenyl)-4-(4-fluoro-2-methylphenyl)-2-{[2-hydroxy-1-(hydroxymethyl)ethyl]-amino}pyrido[2,3-d]pyrimidin-7(8H)-one, and salts thereof.

### FIELD OF THE INVENTION

The present invention relates to a novel process, tablet formulations, polymorphic forms, and to a sustained-release tablet composition for oral delivery of pyrido[2,3-d]pyrimidin-7-one derivatives, exemplified by a water soluble salt of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one.

### BACKGROUND OF THE INVENTION

Many active pharmaceutical agents, including drugs and prodrugs, have been formulated as orally deliverable dosage forms providing sustained release (otherwise known as slow release, extended release or modified release) of such agents over a period of time effective to permit once daily administration. A well-known system for formulating such dosage forms involves a matrix comprising a hydrophilic polymer wherein the active agent is dispersed; the active agent is released over a period of time in the gastrointestinal tract upon dissolution or erosion of the matrix. Sustained-release dosage forms comprising such a matrix system are conveniently prepared as compressed tablets, described herein as “matrix tablets”.

Drugs and prodrugs having relatively low solubility in water, present challenges to the formulator wishing to provide a sustained-release dosage form. The compound 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one is a p38 kinase inhibitor useful in treatment of p38 kinase mediated diseases. A genus covering this compound, uses and methods of synthesis may be found in International Application Number: PCT/US01/50493, International Published Number WO 02/059083 A2 published on Aug. 1, 2002, including page 13, lines 31 to 36 which lists specific salts of the genus of Formula (I), whose entire disclosure is incorporated by reference herein.

A twice daily dosing regimen for immediate-release 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one tablets is currently in development, but patient compliance would be much improved if a once-daily regimen were possible. A once-daily regimen would be especially useful in enhancing compliance among elderly patients.

Amongst the many patents and applications covering erodable matrix tablets are U.S. Pat. No. 6,197,339 disclosing a sustained-release tablet comprising a pharmaceutically active agent, (R)-5,6-dihydro-5-(methylamino)-4H-imidazo[4,5-ij]-quinolin-2(-1H)-one (Z)-2-butenedioate (1:1) (sumanirole maleate) in a matrix comprising hydroxypropylmethylcellulose (HPMC) and starch. Starches disclosed to be suitable therein include pregelatinized starch.

US 2004/0192690 discloses sustained release formulations of lamotrigine, or a pharmaceutically acceptable derivative thereof, including matrix tablets formulated with HPMC, as well as other modified release formulations.

It is an object of the present invention therefore to provide a sustained-release tablet composition of a water-soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one that is suitable for once-daily oral administration.

It is also an object of the present invention to provide for improved synthesis of key intermediates in the processes of making 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, suitable for commercial development.

### SUMMARY OF THE INVENTION

One embodiment of the invention provides for a pharmaceutical composition in a form of an orally deliverable tablet comprising a water-soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, in particular the tosylate salt.

One embodiment of the invention provides for a pharmaceutical composition in a form of an orally deliverable modified release tablet comprising a water-soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one. In one embodiment the water soluble salt is the tosylate salt. In another embodiment the tablet provides for day-long therapeutic effect in a mammal when administered once daily.

Another embodiment of the invention is a pharmaceutical composition in a form of an orally deliverable modified release tablet comprising 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one 4-methylbenzenesulfonate (tosylate salt).

Another embodiment of the invention is a formulation of a water soluble salt of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one in a hydrophilic matrix tablet. In one embodiment the water soluble salt is the tosylate salt.

It is also an object of the invention to provide a modified release composition comprising a water soluble salt of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one, having sufficient hardness to withstand a high-speed tableting operation, in particular to resist erosion during application of a coating layer if one is necessary.

Another embodiment of the present invention is the novel tosylate salt of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one; pharmaceutical compositions comprising the tosylate salt and a pharmaceutically acceptable carrier or diluent, and the use of the tosylate salt for the treating a condition or disease state mediated by p38 kinase activity or mediated by cytokines produced by the activity of the p38 kinase.

Another embodiment of the present invention are the novel polymorphic Forms, Forms 1 to 4 of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate, pharmaceutical compositions comprising these polymorphic forms, alone or in combination or mixtures thereof, and a pharmaceutically acceptable carrier or diluent; and the use of these polymorphic forms of the tosylate salt for treating a condition or disease state mediated by p38 kinase activity or mediated by cytokines produced by the activity of the p38 kinase.

Another embodiment of the present invention are the novel intermediates of Formula (II) shown below:

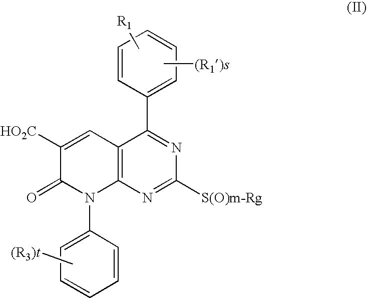

wherein
R₁ is independently selected from hydrogen, C(Z)N(R_{10′})(CR₁₀R₂₀)ᵥR_{b}, C(Z)O(CR₁₀R₂₀)ᵥR_{b}, N(R_{10′})C(Z)(CR₁₀R₂₀)ᵥR_{b}, N(R_{10′})C(Z)N(R_{10′})(CR₁₀R₂₀)ᵥR_{b}, or N(R_{10′})OC(Z)(CR₁₀R₂₀)ᵥR_{b};
R_{1′} is independently selected at each occurrence from hydrogen, halogen, C₁₋₄ alkyl, halo-substituted-C₁₋₄ alkyl, cyano, nitro, (CR₁₀R₂₀)_{v′}NR_{d}R_{d′}, (CR₁₀R₂₀)_{v′}C(O)R₁₂, SR₅, S(O)R₅, S(O)₂R₅, or (CR₁₀R₂₀)_{v′}OR₁₃;
R₃ is independently selected at each occurrence from hydrogen, halogen, C₁₋₄alkyl, or halosubstituted C₁₋₄alkyl;
R₄ and R₁₄ are each independently selected at each occurrence from hydrogen or C₁₋₄ alkyl, or R₄ and R₁₄ together with the nitrogen to which they are attached form a heterocyclic ring of 5 to 7 members, which ring optionally contains an additional heteroatom selected from NR₉;
R₅ is independently selected at each occurrence from hydrogen, C₁₋₄ alkyl, C₂₋₄ alkenyl, C₂₋₄ alkynyl or NR₄R₁₄, excluding the moieties SR₅ being SNR₄R₁₄, S(O)₂R₅ being SO₂H and S(O)R₅ being SOH;
R₉ and R_{9′} are independently selected at each occurrence from hydrogen, or C₁₋₄ alkyl;
R₁₂ is independently selected at each occurrence from hydrogen, C₁₋₄ alkyl, halo-substitutedC₁₋₄ alkyl, C₂₋₄ alkenyl, C₂₋₄ alkynyl, C₃₋₇cycloalkyl, C₃₋₇cycloalkylC₁₋₄ alkyl, C₅₋₇cycloalkenyl, C₅₋₇cycloalkenylC₁₋₄alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R₁₃ is independently selected at each occurrence from hydrogen, C₁₋₄ alkyl, halo-substituted C₁₋₄ alkyl, C₂₋₄ alkenyl, C₂₋₄ alkynyl, C₃₋₇ cycloalkyl, C₃₋₇cycloalkyl C₁₋₄ alkyl, C₅₋₇ cycloalkenyl, C₅₋₇cycloalkenyl C₁₋₄ alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R_{d} and R_{d′} are each independently selected at each occurrence from hydrogen, C₁₋₄ alkyl, C₃₋₆cycloalkyl, C₃₋₆cycloalkyl C₁₋₄alkyl moiety, and wherein each of these moieties, excluding hydrogen, may be optionally substituted; or R_{d} and R_{d′} together with the nitrogen which they are attached form an optionally substituted heterocyclic ring of 5 to 6 members, which ring optionally contains an additional heteroatom selected from oxygen, sulfur or NR_{9′};
R_{b} is hydrogen, C₁₋₁₀ alkyl, C₃₋₇ cycloalkyl, C₃₋₇ cycloalkyl C₁₋₁₀ alkyl, aryl, arylC₁₋₁₀alkyl, heteroaryl, heteroarylC₁₋₁₀ alkyl, heterocyclic, or heterocyclylC₁₋₁₀ alkyl moiety, which moieties, excluding hydrogen, may all be optionally substituted;
R_{g} is C₁₋₁₀ alkyl, or aryl;
m is 0 or an integer having a value of 1, or 2;
s is an integer having a value of 1, 2, 3 or 4; and
t is an integer having a value of 1, 2, 3 or 4.
v is 0 or an integer having a value of 1 or 2;
v′ is independently selected at each occurrence from 0 or an integer having a value of 1 or 2;
Z is independently selected from oxygen or sulfur;
R₁₀ and R₂₀ are independently selected at each occurrence from hydrogen or C₁₋₄ alkyl; and
R_{10′} is independently selected at each occurrence from hydrogen or C₁₋₄alkyl.

Another embodiment of the present invention is the novel process of making a compound of Formula (II) by cyclization of a compound of Formula (IV)

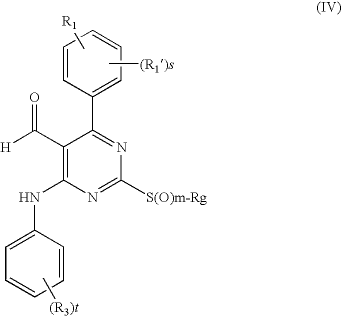

wherein R₁, R_{1′}, R₃, s and t are as described above for Formula (II), m is 0, 1 or 2 and R_{g} is a C₁₋₁₀ alkyl or aryl, with a condensation agent selected from meldrums acid or malonic acid, in an organic solvent, and with a base to yield a compound of Formula (II).

Another aspect of the invention is the novel decarboxylation of a compound of Formula (II) with a thioacid, or a salt of a thioacid, to yield a compound of Formula (III), as shown below:

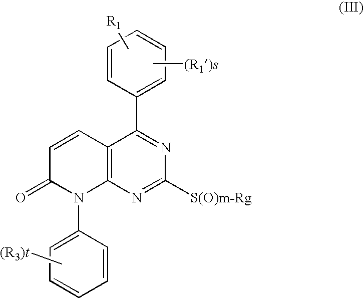

wherein
R₁ is independently selected from hydrogen, C(Z)N(R_{10′})(CR₁₀R₂₀)ᵥR_{b}, C(Z)O(CR₁₀R₂₀)ᵥR_{b}, N(R_{10′})C(Z)(CR₁₀R₂₀)ᵥR_{b}, N(R_{10′})C(Z)N(R₁₀)(CR₁₀R₂₀)R_{b}, or N(R_{10′})OC(Z)(CR₁₀R₂₀)ᵥR_{b};
R_{1′} is independently selected at each occurrence from hydrogen, halogen, C₁₋₄ alkyl, halo-substituted-C₁₋₄ alkyl, cyano, nitro, (CR₁₀R₂₀)_{v′}NR_{d}R_{d′}, (CR₁₀R₂₀)ᵥ, C(O)R₁₂, SR₅, S(O)R₅, S(O)₂R₅, or (CR₁₀R₂₀)ᵥ, OR₁₃;
R₃ is independently selected at each occurrence from hydrogen, halogen, C₁₋₄alkyl, or halosubstituted C₁₋₄alkyl;
R₄ and R₁₄ are each independently selected at each occurrence from hydrogen or C₁₋₄ alkyl, or R₄ and R₁₄ together with the nitrogen to which they are attached form a heterocyclic ring of 5 to 7 members, which ring optionally contains an additional heteroatom selected from NR₉;
R₅ is independently selected at each occurrence from hydrogen, C₁₋₄ alkyl, C₂₋₄ alkenyl, C₂₋₄ alkynyl or NR₄R₁₄, excluding the moieties SR₅ being SNR₄R₁₄, S(O)₂R₅ being SO₂H and S(O)R₅ being SOH;
R₉ and R_{9′} are independently selected at each occurrence from hydrogen, or C₁₋₄ alkyl;
R₁₂ is independently selected at each occurrence from hydrogen, C₁₋₄ alkyl, halo-substitutedC₁₋₄ alkyl, C₂₋₄ alkenyl, C₂₋₄ alkynyl, C₃₋₇cycloalkyl, C₃₋₇cycloalkylC₁₋₄ alkyl, C₅₋₇cycloalkenyl, C₅₋₇cycloalkenylC₁₋₄alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R₁₃ is independently selected at each occurrence from hydrogen, C₁₋₄ alkyl, halo-substituted C₁₋₄ alkyl, C₂₋₄ alkenyl, C₂₋₄ alkynyl, C₃₋₇ cycloalkyl, C₃₋₇cycloalkyl C₁₋₄ alkyl, C₅₋₇ cycloalkenyl, C₅₋₇cycloalkenyl C₁₋₄ alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R_{d} and R_{d′} are each independently selected at each occurrence from hydrogen, C₁₋₄ alkyl, C₃₋₆cycloalkyl, C₃₋₆cycloalkyl C₁₋₄alkyl moiety, and wherein each of these moieties, excluding hydrogen, may be optionally substituted; or R_{d} and R_{d′} together with the nitrogen which they are attached form an optionally substituted heterocyclic ring of 5 to 6 members, which ring optionally contains an additional heteroatom selected from oxygen, sulfur or NR_{9′};
R_{b} is hydrogen, C₁₋₁₀ alkyl, C₃₋₇ cycloalkyl, C₃₋₇ cycloalkyl C₁₋₁₀ alkyl, aryl, arylC₁₋₁₀alkyl, heteroaryl, heteroarylC₁₋₁₀ alkyl, heterocyclic, or heterocyclylC₁₋₁₀ alkyl moiety, which moieties, excluding hydrogen, may all be optionally substituted;
R_{g} is C₁₋₁₀ alkyl, or aryl;
m is 0 or an integer having a value of 1, or 2;
s is an integer having a value of 1, 2, 3 or 4; and
t is an integer having a value of 1, 2, 3 or 4.
v is 0 or an integer having a value of 1 or 2;
v′ is independently selected at each occurrence from 0 or an integer having a value of 1 or 2;
Z is independently selected from oxygen or sulfur;
R₁₀ and R₂₀ are independently selected at each occurrence from hydrogen or C₁₋₄ alkyl; and
R_{10′} is independently selected at each occurrence from hydrogen or C₁₋₄alkyl.

Another aspect of the present invention is a novel one pot, in situ synthesis to make a compound of Formula (III) as described herein, by cyclization of a compound of Formula (IV) as described herein with a condensation agent selected from meldrums acid or malonic acid, in an organic solvent, and with a base to yield a compound of Formula (II) as described herein, and then decarboxylating a compound of Formula (II) with a thioacid derivative, or a salt of a thioacids derivative, to yield a compound of Formula (III).

### BRIEF DESCRIPTION OF THE DRAWINGS

FIG. 1a provides for dissolution profiles of the formulations of Example 1, as a 2.5 and as a 5 mg immediate release tablet acquired using the paddle apparatus of the USP (USP II, Chapter <711>).

FIG. 1b provides for a dissolution profiles of the formulations of Examples 2 to 4, acquired using the reciprocating cylinder apparatus of the USP (USPIII, Chapter <711>).

FIG. 2 provides for the dissolution profiles for the formulations of Examples 2 to 4, acquired using the basket apparatus of USP I, Chapter <711>.

FIG. 3 demonstrates graphically the pK profiles as obtained in humans for the formulations of Examples 1 to 4. The immediate release formulation is a 7.5 mg dose.

FIG. 4 provides for the dissolution profiles for the formulations of Examples 5 to 6, acquired using the basket apparatus of the USP (USP I, chapter <711>).

FIG. 5 provides XRPD data for polymorphic Form 1 of the 4-methyl-benzenesulphonate (tosylate) salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one.

FIG. 6 provides XRPD data for polymorphic Form 2 of the 4-methyl-benzenesulphonate (tosylate) salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one.

FIG. 7 provides XRPD data for polymorphic Form 3 of the 4-methyl-benzenesulphonate (tosylate) salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one.

FIG. 8 provides XRPD data for polymorphic Form 4 of the 4-methyl-benzenesulphonate (tosylate) salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one.

FIG. 9 provides for a Differential Scanning Calorimetry (DSC) thermogram of Form 1.

FIG. 10 provides for a Differential Scanning Calorimetry (DSC) thermogram of Form 2.

FIG. 11 provides for a Differential Scanning Calorimetry (DSC) thermogram of Form 3.

FIG. 12 provides for a Differential Scanning Calorimetry (DSC) thermogram of Form 4.

FIG. 13 provides for an FT-IR spectrum of Form 1, with data presented as 4000-700 cm⁻¹ (top figure, FIG. 13 (a)) and 2000-700 cm⁻¹ (bottom figure, FIG. 13 (b)).

FIG. 14 provides for an FT-IR spectrum of Form 2, with data presented as 4000-700 cm⁻¹ (top figure, FIG. 14 (a)) and 2000-700 cm⁻¹ (bottom figure, FIG. 14 (b)).

FIG. 15 provides for an FT-IR spectrum of Form 3, with data presented as 4000-700 cm⁻¹ (top figure, FIG. 15 (a)) and 2000-700 cm⁻¹ (bottom figure, FIG. 15 (b)).

FIG. 16 provides for an FT-IR spectrum of Form 4, with data presented as 4000-700 cm⁻¹ (top figure, FIG. 16 (a)) and 2000-700 cm⁻¹ (bottom figure, FIG. 16 (b)).

FIG. 17 provides for a Differential Scanning Calorimetry (DSC) thermogram of amorphous material of the 4-methyl-benzenesulphonate (tosylate) salt of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one; using Perkin Elmer Thermal Analysis, demonstrating a peak of 63.89° C., delta H=3.070 J/gm; area=5.149 mJ; Onset 58.20° C.

### DETAILED DESCRIPTION OF THE INVENTION

There is now provided a sustained-release pharmaceutical composition in a form of an orally deliverable tablet comprising a water-soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, dispersed in a matrix comprising a hydrophilic polymer and additional pharmaceutically acceptable excipients.

Suitable water-soluble pharmaceutically acceptable salts include but are not limited to the tosylate, the hydrochloride, the hydrobromide, and the sulphate. The tosylate is preferred for use in the immediate release (IR) and modified release (MR) dosage forms as disclosed herein.

It will be understood that mention of a water-soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one herein embraces racemates, enantiomers, polymorphs, hydrates and solvates thereof.

The tosylate, hydrochloride, hydrobromide, and the sulphate salt forms have similar solubilities and stability. The sulphate salt is less stable than the other salt forms. The hydrobromide and the tosylate salts appear to have less complicated thermal profiles.

There is further provided a method of treatment of a subject having a condition or disorder for which a p38 kinase inhibitor is indicated, the method comprising orally administering to the subject a sustained-release pharmaceutical composition in a form of a tablet comprising a water-soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one dispersed in a matrix comprising a hydrophilic polymer and additional pharmaceutically acceptable excipients.

The compound 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate is useful for the treatment, including prophylaxis, of a condition or disease state mediated by p38 kinase activity or mediated by cytokines produced by the activity of the p38 kinase.

The compound 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate can be used in the manufacture of a medicament for the prophylactic or therapeutic treatment of any disease state in a human, or other mammal, which is exacerbated or caused by excessive or unregulated cytokine production by such mammal's cell, such as but not limited to monocytes and/or macrophages.

Suitable CSBP/RK/p38 kinase mediated diseases include psoriatic arthritis, Reiter's syndrome, gout, traumatic arthritis, rubella arthritis, acute synovitis, rheumatoid arthritis, rheumatoid spondylitis, osteoarthritis, gouty arthritis and other arthritic condition, sepsis, septic shock, endotoxemia, endotoxic shock, gram negative sepsis, toxic shock syndrome, cerebral malaria, meningitis, ischemic and hemorrhagic stroke, neurotrauma/closed head injury, asthma, adult respiratory distress syndrome, chronic pulmonary inflammatory disease, chronic obstructive pulmonary disease, chronic heart failure, silicosis, pulmonary sarcososis, bone resorption disease, osteoporosis, restenosis, cardiac and brain and renal reperfusion injury, congestive heart failure, coronary arterial bypass grafting (CABG) surgery, thrombosis, glomerulonephritis, chronic renal failure, diabetes, diabetic retinopathy, macular degeneration, graft vs. host reaction, allograft rejection, inflammatory bowel disease, Crohn's disease, ulcerative colitis, irritable bowel syndrome, neurodegenerative disease, muscle degeneration, diabetic retinopathy, Alzheimer's disease, Parkinson's disease, Huntington's disease, amyotrophic lateral sclerosis, epilepsy, multiple sclerosis, macular degeneration, tumor growth and metastasis, angiogenic disease, influenza induced pneumonia, eczema, contact dermatitis, psoriasis, sunburn, conjunctivitis, allergic rhinitis, allergic conjunctivitis, psychiatric disorders, aneurism, stroke, graft vs. host reaction, allograft rejections, systemic cachexia, cachexia secondary to infection or malignancy, cachexia secondary to acquired immune deficiency syndrome (AIDS), malaria, leprosy, infectious arthritis, leishmaniasis, Lyme disease, spondylitis, and non articular inflammatory conditions, for example, herniated/ruptured/prolapsed intervertebral disk syndrome, bursitis, tendonitis, tenosynovitis, fibromyalgic syndrome and, other inflammatory conditions associated with ligamentous sprain and regional musculoskeletal strain, pain, for example that associated with inflammation and/or trauma, thrombosis, angiogenesis, and cancer including breast cancer, colon cancer, lung cancer or prostatic cancer.

P38 inhibitors have also been found to be useful in chronic diseases which have an inappropriate angiogenic component are various ocular neovasularizations, such as diabetic retinopathy and macular degeneration. Other chronic diseases which have an excessive or increased proliferation of vasculature are tumor growth and metastasis, atherosclerosis, and certain arthritic conditions.

Preferred diseases include rheumatoid arthritis, acute or chronic inflammatory disease states such as the inflammatory reaction induced by endotoxin or inflammatory bowel disease, Crohn's disease, ulcerative colitis, irritable bowel syndrome, atherosclerosis, neuropathic pain, chronic obstructive pulmonary disease (COPD), asthma, cystic fibrosis, and multiple myeloma.

Accordingly, the present invention provides a method of treating a CSBP kinase mediated disease in a mammal in need thereof, preferably a human, which comprises administering to said mammal, an effective amount of S-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate.

In order to use 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate in therapy, it will normally be formulated into a pharmaceutical composition in accordance with standard pharmaceutical practice. This invention, therefore, also relates to a pharmaceutical composition comprising an effective, non-toxic amount of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one, tosylate and a pharmaceutically acceptable carrier or diluent. A description of formulations and compositions may be found in WO 02/059083 A2 published on Aug. 1, 2002. A suitable pharmaceutical textbook includes Remington's Pharmaceutical Sciences.

8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate may conveniently be administered by any of the routes conventionally used for drug administration, for instance, orally, topically, parenterally or by inhalation. The tosylate may be administered in conventional dosage forms prepared by combining it with standard pharmaceutical carriers according to conventional procedures. The compound may also be administered in conventional dosages in combination with a known, second therapeutically active compound. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof.

For all methods of use disclosed herein for the tosylate salt, the daily oral dosage regimen will preferably be from about 0.1 to about 30 mg/kg of total body weight, preferably from about 0.5 mg to 15 mg. The daily parenteral dosage regimen about 0.1 to about 30 mg/kg of total body weight, preferably from about 0.5 mg to 15 mg/kg. The daily topical dosage regimen will preferably be from 0.1 mg to 50 mg, administered one to four, preferably two or three times daily. The daily inhalation dosage regimen will preferably be from about 0.01 mg/kg to about 1 mg/kg per day. It will also be recognized by one of skill in the art that the optimal quantity and spacing of individual dosages of the tosylate salt will be determined by the nature and extent of the condition being treated, the form, route and site of administration, and the particular patient being treated, and that such optimums can be determined by conventional techniques.

The term “water-soluble” herein means having solubility of at least about 0.5 mg/ml over the pH range. Unless otherwise specified, “solubility” herein means solubility in water at 20-25° C. at any physiologically acceptable pH, for example at any pH in the range of about 1 to about 8. In the case of a salt, reference herein to solubility in water pertains to the salt, not to the free base form of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one. The compound 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate has been found to have a solubility in water of 0.7 mg/ml at pH 2.9.

The term “orally deliverable” herein means suitable for oral, including peroral and intra-oral (e.g., sublingual or buccal) administration, but tablets of the present invention are adapted primarily for peroral administration, i.e., for swallowing, typically whole or broken, with the aid of water or other drinkable fluid.

A “subject” herein is an animal of any species, preferably mammalian, most preferably human. Conditions and disorders in a subject for which a particular agent is said herein to be “indicated” are not restricted to conditions and disorders for which the agent has been expressly approved by a regulatory authority, but also include other conditions and disorders known or believed by a physician to be amenable to treatment with the agent.

“Treatment” herein embraces prophylactic treatment unless the context requires otherwise. The term “treatment” as used herein includes the treatment of established disorders and also includes the prophylaxis thereof unless the context requires otherwise.

When used herein the term “pharmaceutically acceptable salt” means a salt which upon administration to the recipient such a human is capable of providing (directly or indirectly) the active compound or an active metabolite thereof to said human.

As used herein, the term “sustained release” or “modified release” refers to the gradual but continuous release over any extended period of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one after oral ingestion, over a 24 hour period in a mammal, preferably a human. The release starts when the formulation reaches the stomach and starts to disintegrate/dissolve/erode. The release will continue over a period of time and may continue throughout the small intestine and after the formulation reaches the large intestine up through the colon.

Suitably, for a composition corresponding to Example 2, an in vivo release between 1 and 2 hours of about 15 to about 55% is anticipated, with preferably a release of about 20 to 50%, and more suitably about a 25 to 45% release. Between 2 and 3 hours a release of about 35 to about 75% release is anticipated with preferably a 40 to 70% release, and more preferably a 45 to 65% release. Between 3 and 4 hours, a release of about 60 to about 100% is anticipated, preferably a 65 to 95% release, and more preferably a 70 to 90% release.

Suitably, for a composition corresponding to Example 3, an in vivo release between 1 and 2 hours of about 5 to about 35% is anticipated, with preferably a release of about 10 to 35%, and more suitably between 15 to 30% release. Between 2 and 4 hours a release of about 20 to about 65% is anticipated, with preferably a 25 to 60% release, and more preferably a 25 to 55% release. Between 5 and 7 hours, a release of about 65 to about 100% is anticipated, preferably a 70 to 95% release, and more preferably a 70 to 90% release.

Suitably, for a composition corresponding to Example 4, an in vivo release between 1 and 3 hours of about 0 to about 30% is anticipated, with preferably a release of 5 to 30%, more preferably a release of 10 to 25%. Between 4 and 8 hours a release of about 25 to 65% release is anticipated, with preferably a 30 to 55% release, and more preferably 30 to 50% release. Between 12 and 16 hours, a release of about 70 to about 100% is anticipated, preferably 75 to 95% release, and more preferably 75 to 90% release.

Suitably for a composition corresponding to Example 5, an in vivo release between 1 and 3 hours of about 0 to 30% is anticipated, with preferably a release of 5 to 30%, more preferably a release of 10 to 25%. Between 4 and 8 hours a release of about 20 to 60% release is anticipated, with preferably a 25 to 50% release, and more preferably 30 to 45% release. Between 12 and 16 hours, a release of about 60 to 100% is anticipated, preferably 65 to 95% release, and more preferably 70 to 90% release.

Suitably for a composition corresponding to Example 6, an in vivo release between 1 and 3 hours of about 0 to 25% is anticipated, with preferably a release of 5 to 20%, more preferably a release of 10 to 20%. Between 4 and 8 hours a release of about 15 to 50% release is anticipated, with preferably a 20 to 45% release, and more preferably a 25% to 45% release. Between 14 and 18 hours, a release of about 60 to 100% is anticipated, preferably 65 to 95% release, and more preferably 70 to 90% release.

When used herein “substantially all” means more than 85%, preferably more than 90%.

As used herein, the term “substantially pure” when used is reference to the tosylate salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one refers to a product which is greater than about 90% pure. Preferably, “substantially pure” refers to a product which is greater than about 95% pure, more preferably greater than about 97% pure, and most preferable about 99% pure. This means the product does not contain any more than about 10%, 5%, 3% or 1% respectively of any other compound, or impurity or any other polymorphic form of the tosylate salt than the one desired, e.g. Form 1, 2, 3, or 4.

Administration of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, or a pharmaceutically acceptable salt thereof, over a time period, suitably up to 18 hours, delivers it gradually to the sites where it is readily absorbed but with a slower rise in serum concentrations and reduced post-dosing peaks to mitigate potential dosing related adverse events (AE's) and yet provide sufficient minimum plasma/serum concentrations (Cmin) to maintain efficacy.

A formulation which achieves an area under the curve (AUC) equivalent to the conventional instant/immediate release (IR) tablet (90% confidence interval (CI) for the geometric least squares (GLS) mean ratio should fall within the range 80-125% compared to the reference IR product) is termed “bioequivalent”. A sustained release formulation would likely not be deemed by the Food and Drug Administration (FDA) to be bioequivalent to the IR tablets if the points estimate and the associated 90% Confidence Interval for Cmax do not fall within the limit of 80-125% relative to the IR product with the AUC remaining within the 80-125% range compared with the reference IR product. Suitably, the formulations will be formulated such that the release of the active substance is predominantly in the stomach, small intestine and into the colon.

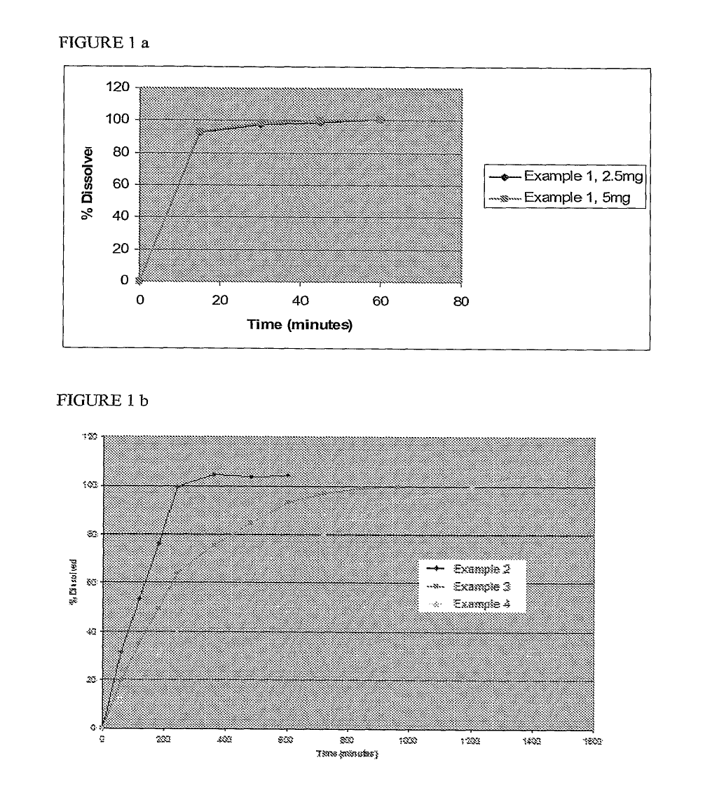

A conventional, immediate release tablet dosage form of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate is expected to dissolve 80% within 45 minutes. The dissolution profile was measured in a standard dissolution assay, for instance <724> Dissolution Test, paddle apparatus, (USP II, chapter <711>), at 37.0+/−0.5 C.°, using 0.01M hydrochloric acid (500 ml) and a rotation speed of 75 rpm. The profiles for a 2.5 and a 5 mg IR tablet as shown in Example 1 are illustrated in FIG. 1a.

The sustained release formulation when administered in vivo may provide an in vivo “Area Under the Curve” (AUC) value which is equivalent to that of the existing instant release IR tablet, for instance at least 80%, preferably at least 90% to 110%, more preferably about 100%, but not exceeding 125% of that of the corresponding dosage of a water soluble salt of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one, taken as a conventional (immediate release) formulation, over the same dosage period, thereby maximizing the absorption of a water soluble salt of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one from the sustained release formulation. Suitably, the water soluble salt used in the immediate release or the sustained release formulation is the tosylate salt of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one.

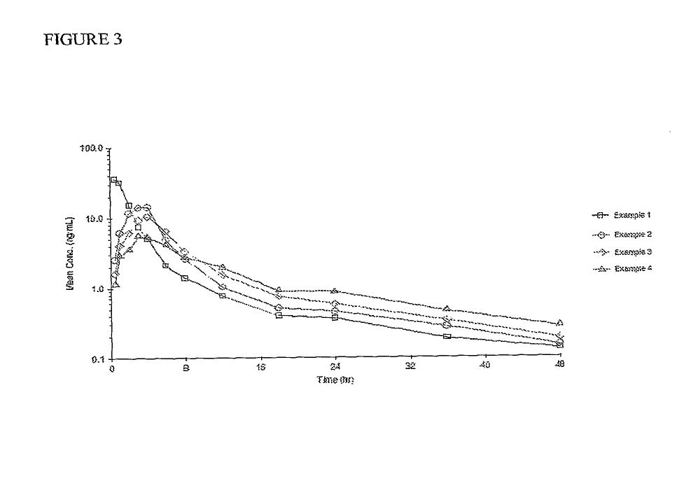

The pharmacokinetic profile for a dosage of the present invention may be readily determined from a single dosage bioavailability study in human volunteers. Plasma concentrations of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate may then be readily determined in blood samples taken from patients according to procedures well known and documented in the art. Similar pharmacokinetic profiles have been determined for formulations corresponding to Examples 1 to 4 herein, shown in FIG. 3.

Similarity factor (f2) is a recognized method for the determination of the similarity between the dissolution profiles of a reference and a test compound. Similarity factor (f2) is a logarithmic transformation of the sum of squared error. The similarity factor (f2) is 100 when the test and reference profiles are identical and approaches zero as the dissimilarity increases. The similarity factor has also been adapted to apply to the determination of the similarity between the dissolution profiles of a reference and test compound as they relate to modified release formulations, such as those exemplified herein.

The f2 similarity factor has been adopted in the SUPAC guidelines and by the FDA guidance on dissolution testing of immediate release dosage forms (FDA Guidance for Industry, Dissolution Testing of Immediate Release Solid Oral Dosage Forms, FDA, (CDER), August 1997 (Dissolution Tech. 4, 15-22, 1997).

The f2 similarity factor has been adopted in the FDA in the SUPAC guidelines for modified release solid oral dosage forms (FDA Guidance for Industry, SUPAC-MR: Modified Release Solid Oral Dosage Forms, Scale-Up and Postapproval Changes: Chemistry, Manufacturing, and Controls; In Vitro Dissolution Testing and In Vivo Bioequivalence Documentation; CDER; September 1997). The FDA Guidance for Industry on Dissolution Testing of Immediate Release Solid Oral Dosage Forms may be found at http://www.fda.gov/cder/guidance/1713bp1.pdf.

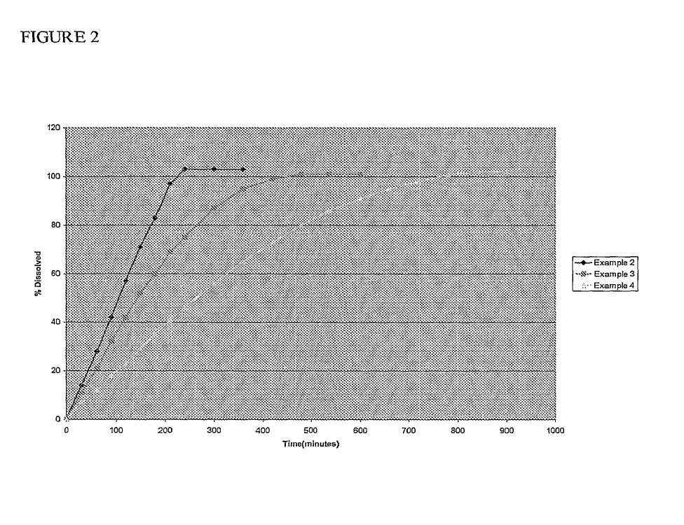

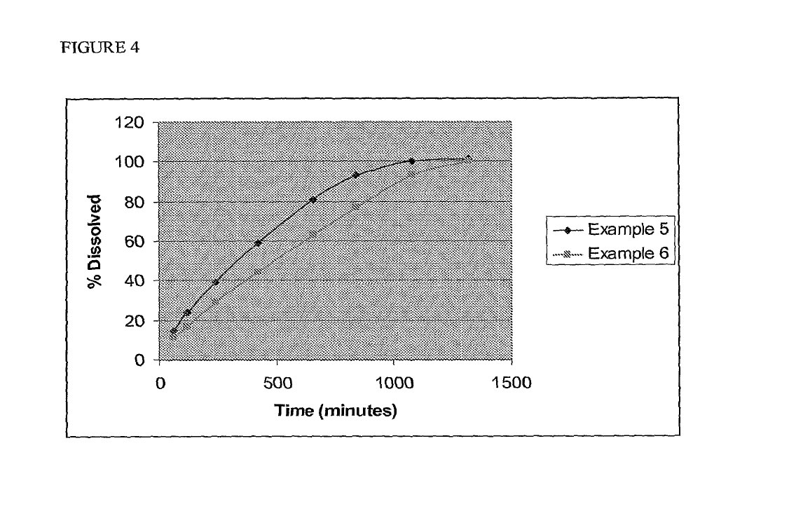

One embodiment of the invention is a sustained release composition comprising a water-soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one which has an in vitro dissolution profile generated using the basket apparatus of the USP (USP I, Chapter <711>) wherein the similarity factor (f2) is between 50 and 100 when calculated using one of the examples in FIG. 2 or FIG. 4 as the reference profile.

The person skilled in the art will appreciate that a therapeutically effective amount to be determined will depend on the patient's age, size, severity of disease and other medication.

Suitably, the sustained release formulations are a (un)coated or coated tablet or caplet.

One aspect of the invention is a formulation comprising 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one or a pharmaceutically acceptable derivative thereof, and a release retarding excipient, which allows for sustained release of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one or a pharmaceutically acceptable derivative thereof. Suitably the pharmaceutically acceptable derivative thereof is a water soluble salt, and the water soluble salt is preferably a tosylate salt.

Suitable release retarding excipients include release-retarding polymers which may be swellable or not in contact with water or aqueous media such as the stomach contents; polymeric materials which form a gel on contact with water or aqueous media; polymeric materials which have both swelling and gelling characteristics in contact with water or aqueous media and pH sensitive polymers, for instance polymers based upon methacrylic acid copolymers such as the Eudragit™ polymers, for example Eudragit L™ which may be used either alone or with a plasticizer.

These sustained release formulations are often referred to in the art, as “matrix formulations” where by the drug is incorporated into a polymer matrix system, preferably a one which hydrates in the environmental fluids of the intestinal tract, and is released from the matrix via diffusion or erosion.

Release retarding polymers which may be swellable or not include, inter alia, cross-linked sodium carboxy methylcellulose, hydroxypropyl cellulose, cross-linked hydroxypropyl cellulose, hydroxyethyl cellulose, high-molecular weight hydroxypropyl methylcellulose, carboxymethylamide, potassium methacrylatedivinylbenzene co-polymer, polymethylmethacrylate, cross-linked polyvinylpyrrolidone, hydroxyethyl cellulose, or high-molecular weight polyvinylalcohols etc., and combinations or mixtures thereof.

A release retarding polymer may also be referred to herein as a hydrophilic polymer which is a polymeric material having a sufficient number and distribution of hydrophilic substituents such as hydroxy and carboxy groups to impart hydrophilic properties to the polymer as a whole. Suitable hydrophilic polymers include, without limitation, methylcellulose, hydroxypropylmethylcellulose (HPMC or hypromellose), carmellose (carboxymethylcellulose) sodium, xanthan gum and carbomer (polyacrylic acid). More than one such polymer, in combinations or mixtures thereof can optionally be used.

In one embodiment of the invention HPMC is the hydrophilic polymer.

Release retarding gellable polymers include methyl cellulose, carboxy methylcellulose, low-molecular weight hydroxypropyl methylcellulose, hydroxyethyl cellulose, low-molecular weight polyvinylalcohols, polyoxyethyleneglycols, non-cross linked polyvinylpyrrolidone, or xanthan gum etc., and combinations or mixtures thereof. More than one such polymer, in combinations or mixtures with other exemplified polymers herein can optionally be used.

Release retarding polymers simultaneously possessing swelling and gelling properties include medium-viscosity hydroxypropylmethylcellulose and medium-viscosity polyvinylalcohols.

Suitably, the release retarding polymer used has a molecular weight in the range 5 to 95 thousand, more preferably in the range 10 to 50 thousand.

The release-retarding polymer is suitably present in the formulation from about 15 to about 50% w/w. In another embodiment of the invention the release-retarding polymer is present in an amount of about 20% to about 45% w/w.

One aspect of the invention is that the release-retarding polymer is a commercially available grade of hydroxypropylmethyl cellulose, or is hydroxyethyl cellulose.

Examples of suitable commercial polymers which may be used include but are not limited to Methocel K4M™, Metolose 90SH™, Methocel E5M™, Methocel E50™, Methocel E4M™, Methocel E10M™, Methocel E100M™, Methocel K15M™, Methocel K100M™ and Methocel K100LV™, or POLYOX WSR N-80, Walocel HM 3PA 2910™ and Walocel HM 15PA 2910™, and combinations or mixtures thereof.

When the release retarding polymer is hydroxypropyl methylcellulose, it is suitably present in an amount of from about 15% to about 50%, dependent upon the HPMC grade. In one embodiment the HPMC is present in an amount of about 20% to about 45% w/w, again dependent upon the HPMC grades used, and which may be a blend of available grades. As noted, various types and grades of HPMC are available. The hydroxypropyl methylcellulose may be hydroxypropyl methylcellulose type 2208, suitably meeting specifications set forth in a standard pharmacopoeia such as USP 28. HPMC type 2208 contains 19-24% by weight methoxy and 4-12% by weight hydroxypropoxy substituents. HPMC's have nominal viscosity ranging from about 100 to about 100,000 GP; illustratively a suitable HPMC type 2208 is one having a nominal viscosity of about 4,000 cP, with a measured viscosity of about 3,000 to about 5,600 cP. Such an HPMC is available, for example, as Methocel K4M Premium from Dow Chemical Co., and substantially equivalent products are available from other manufacturers, for example, as Metolose 90 SH from Shinetsu.

Other hydroxypropyl methylcellulose polymers include type 2208 at USP 100 cP, hydroxypropyl methylcellulose 2208 USP 4,000 cP, hydroxypropyl methylcellulose 2208 USP 15,000 cP, hydroxypropyl methylcellulose 2208 USP 100,000 cP, hydroxypropyl methylcellulose 2910 USP 4,000 cP, hydroxypropyl methylcellulose 2910 USP 10,000 cP, or mixtures thereof.

In one embodiment it is preferred that the hydroxypropyl methylcellulose be hydroxypropyl methylcellulose 2208 USP 4,000 cP or hydroxypropyl methylcellulose 2910 USP 4,000 cP. The hydroxypropyl methylcellulose can be any of the hydroxypropyl methylcelluloses individually or as a mixture. The centepoid values (2% in water at 20 C) for HPMC K100 and K4M are 80-120 and 3000-5600 respectively.

Other known release-retarding polymers, also referred to herein as a “natural release-retarding polymer”, which may be incorporated include hydrocolloids such as natural or synthetic gums, cellulose derivatives other than those listed above, carbohydrate-based substances such as acacia, gum tragacanth, locust bean gum, guar gum, agar, pectin, carragenen, soluble and insoluble alginates, chitosans, carboxypolymethylene, casein, zein, and the like, and proteinaceous substances such as gelatin. More than one such polymer, in combinations or mixtures with other exemplified polymers herein can optionally be used. These polymers may be used alone or in combination with the hydrophilic or gellable polymers.

The natural release-retarding polymer is optionally present in the formulation in an amount of about 0.1% to about 50% w/w.

One embodiment of the invention is the use of release-retarding polymers Methocel E4M Grade, and/or Methocel K100LV. In one embodiment of the invention when the release-retarding polymer is Methocel K100LV or equivalent grade, the polymer is suitably present at about 15 to about 50% w/w. In one embodiment the polymer is present from about 20% to about 45%. In another embodiment from about 30 to about 45% w/w.

In another embodiment of the invention when the release-retarding polymer is Methocel K4M, the polymer is suitably present from about 15 to about 50% w/w. In one embodiment the polymer is present from about 20% to about 45%. In another embodiment the polymer is present from about 20% to about 29%.

The sustained release formulation may also include diluents including but not limited to bulk sweeteners, such as a sugar, e.g. dextrose, sucrose, lactose, confectionery sugar, or powdered sugar, and combinations or mixture thereof; or a polyol, such as mannitol, sorbitol, xylitol, maltitol, maltose and polydextrose, and combinations or mixtures thereof. The diluent may also suitably be a combination of at least one bulk sweetener and at least one polyol. Such diluents may be present in an amount of about 20 to about 70% by weight. In one embodiment of the invention the diluent is present from about 25 to about 55% w/w.

The formulation may also include a binding agent, such as a starch. Suitably starches for use herein may be from any suitable botanical source, for example corn, wheat, rice, tapioca, potato, etc., and include modified versions thereof, such as modified corn starch, modified wheat starch, Starch 1500, or pregelatinized starch; alone or in combination or mixtures thereof. Some starches have a relatively high ratio of amylose to amylopectin, containing for example at least about 20%. Pregelatinized starch is a type of modified starch that has been processed to render the starch more flowable and directly compressible. Partially or wholly pregelatinized starches can be used. The starch is present in an amount from about 3% to about 10% w/w of the tablet weight.

Other suitable binding agents include low viscosity cellulosic derivatives, including but not limited to a carbomer, hydroxypropylmethylcellulose (HPMC), hydroxypropylcellulose (HPC), MCC, carboxymethylcellulose (CMC), hydroxyethylcellulose (HEC), or methylcellulose (MC), in combination or mixtures thereof. The cellulosic is present in an amount from about 1% to about 10% of the tablet weight.

Another suitable binding agent is a natural gum such as gum arabic, accacia, carrageenan, guar gum, or tragacanth, in combination or mixtures thereof. The gum is present in an amount from about 1% to about 10% of the tablet weight.

Other alternative binding agents include povidone (PVP), poloxamer, PEG, or a polymethacrylate, in combination or mixtures thereof. The alternative binding agents are present in an amount from about 1% to about 10% of the tablet weight. It is also recognized that the bulk sweeteners noted above may also function as a binding agent, such as maltodextrin, mannitol, sorbitol, or polydextrose. All of the above noted binding agents may suitably be used in combination or mixtures with each other as may be determined by the skilled artisan.

It is also recognized that some of the binding agents may also be present as a swellable polymer or as a natural release retarding polymer, alone or in combination with other binding agents.

The sustained release formulation may also include lubricants to enhance release of a tablet from apparatus on which it is formed, for example by preventing adherence to the face of an upper punch (“picking”) or lower punch (“sticking”). Suitable lubricants include magnesium stearate, calcium stearate, sodium stearate, canola oil, glyceryl palmitostearate, hydrogenated vegetable oil, magnesium oxide, mineral oil, poloxamer, polyethylene glycol, polyvinyl alcohol sodium benzoate, sodium lauryl sulfate, sodium stearyl fumarate, stearic acid, Cab-O-Sil, Syloid, talc, hydrogenated vegetable oil, zinc stearate and the like. Suitably, the lubricant is present in an amount of about 0.1% to about 2.5% w/w of the tablet. In one embodiment the lubricant is present in an amount of about 0.5% by weight of the tablet. In another embodiment magnesium stearate is the lubricant present in an amount of about 0.1% to about 2.5% w/w of the tablet.

The sustained release formulation may also include compression aids, such as microcrystalline cellulose; calcium phosphate (dihydrate or anhydrous), mannitol, lactose or sorbitol. Such compression aids may be present in an amount of about 0 to about 80%, suitably from about 10 to about 80% by weight. It is also recognized that some of the diluents may also function as a compression aid, such as maltodextrin, mannitol, sorbitol, or polydextrose.

The sustained release formulation may further comprise disintegrants or superdisintegrants, such as cross-linked polyvinylpyrrolidone (CLPVP) and sodium starch glycolate, and combinations or mixtures thereof; alternatively povidone (polyvinylpyrrolidone). Such disintegrants may be present in an amount of about 0 to about 70% by weight. In one embodiment of the invention from about 1% to about 70% w/w.

A flow aid or glidant can be used to improve powder flow properties prior to and during tableting and to reduce caking. Suitable glidants include colloidal silicon dioxide, magnesium trisilicate, powdered cellulose, talc, tribasic calcium phosphate and the like, optionally in combination or mixtures thereof. A glidant may be present in an amount up to about 2%, preferably from about 0.2% to about 0.6% by weight of the tablet. In one embodiment of the invention the glidant is colloidal silicon dioxide.

Typically, the sustained release formulation comprises from about 1 to 20% by weight of water soluble salt; from 0 to about 70% by weight of diluent/compression aid; from about 0.1 to about 2.5% by weight of lubricant; and from about 15 to about 50% release retarding excipient.

A further aspect of the invention is a formulation comprising 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one or a pharmaceutically acceptable salt thereof and a release retarding coating on one or more of the outer surfaces of the tablet. In one embodiment of the invention the pharmaceutically acceptable salt is a water soluble salt, and is preferably a tosylate salt.

The release retarding coating may be a film coat, which may be compression or spray dried, and may act as a semi permeable barrier thereby allowing diffusion control of drug release by water insoluble polymer, or a partially water-soluble polymer. Alternatively the film coating may control the dissolution rate. Such film coating may, for example, be composed of polymers which are either substantially or completely impermeable to water or aqueous media, or are slowly erodable in water or aqueous media or biological liquids and/or which swell in contact with water or aqueous media or biological liquids. Suitably, the film coat should be such that it retains these characteristics at least until complete or substantially complete transfer of the active material content to the surrounding medium. Such film coated tablets are also referred to as functional film coated tablets.

Suitable polymers for the film coating include but are not limited to acrylates, methacrylates, copolymers of acrylic acid or its esters, celluloses and derivatives thereof such as ethylcelluloses, cellulose acetate propionate, polyethylenes and polyvinyl alcohol, etc. Film coats comprising polymers which swell in contact with water or aqueous media may swell to such an extent that the swollen layer forms a relatively large swollen mass, the size of which delays its immediate discharge from the stomach into the intestine. Film coats may typically have an individual thickness of 2 microns to 10 microns.

Suitable polymers for film coats which are relatively impermeable to water include hydroxypropyl methylcellulose polymers for example the Methocel™ series of polymers mentioned above, for example Methocel K100M, Methocel K15M; Eudragit™ family of polymers, Aquacoat™ and used singly or combined, or optionally combined with an Ethocel™ polymer. Another polymer suitable for coating is SURELEASE™ which is aqueous ethylcellulose dispersion. This can be obtained from COLORCON a division of Berwind Pharmaceuticals Services, Inc. Additionally, a mixture of SURELEASE polymer or other suitable partially permeable polymer, and a pore forming material for example OPADRY™ clear (YS-2-7013), again obtainable from COLORCON, can be used. One suitable range of film coating polymers is from about 3 to about 5% w/w of coating on a tablet.

The coating, if present, can optionally contain additional pharmaceutically acceptable excipients such as plasticizers, dyes, etc. One suitable plasticizer is hydrogenated castor oil may be combined with the coating polymer. The film coating may also include conventional binders, fillers, lubricants, colorants such as iron oxides or organic dyes and compression aids etc such as Polyvidon K30™, magnesium stearate, and silicon dioxide, e.g. Syloid 244™.

Matrix tablets as described above can be compression or spray coated with an aqueous solution of the polymer to produce a film coat. Coating can take place in any standard coating machine known to the person skilled in the art, for example a Vector™ machine.

Tablets can be of any suitable size and shape, for example round, oval, polygonal or pillow-shaped, elliptical, shield or capsule shape, shallow to deep convex and optionally bear nonfunctional surface markings. Preferably, the tablet is round or oval shaped, standard convex. Tablets of the invention can be packaged in a container, accompanied by a package insert providing pertinent information such as, for example, dosage and administration information, contraindications, precautions, drug interactions and adverse reactions.

In one embodiment, the sustained release formulation comprises;
a) about 2.5 to about 25% by weight 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, or a water soluble salt or a pharmaceutically acceptable salt thereof;
b) about 15 to about 50% by weight release retarding polymer;
d) about 25 to about 55% weight diluent;
c) 0 to about 40% by weight compression aid; and
e) about 0.1 to about 2.5% by weight lubricant.

Suitably, the release retarding polymer is HPMC Type 2208 or 2910. In one embodiment of the invention the release retarding polymer is Methocel K100LV. In another embodiment of the invention when the release retarding polymer is HPMC Type 2208, it is present in an amount of about 30 to about 45% w/w. In another embodiment of the invention the when the HPMC Type 2208 is present in an amount of about 20 to about 25% w/w.

The amount of the water soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one present in a composition of the invention is sufficient to provide a daily dose to be administered at one time daily. Preferably, the full daily dose is delivered in a single tablet.

An amount of water soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one present in the tablet is suitably from about 0.5 to about 30 mg per tablet. In one aspect of the invention the water soluble salt of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one is present in about 1% to about 20% by weight of the tablet. Suitably, the water soluble salt is the tosylate salt.

In another embodiment of the invention the water soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one is present in an amount of about 0.5 mg, 0.75 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg to about 30 mg per unit dosage form, suitably a tablet. In one embodiment of the invention, the water soluble salt is the tosylate salt.

### General Bulk Method Preparation:

The tablet may be made by either direct compression or wet granulation, both processes are well known to those skilled in the art. If conventional direct compression is used, the active ingredient, and the excipients except for the lubricant, are first transferred to an appropriate size blending drum, and blended. The mixture is screened/sieved and then further blended. Magnesium stearate, or other suitable lubricant is added and further blended. The lubricated mixture is compressed into tablets of desired weight and physical specifications by methods known to those skilled in the art.

Alternatively, if conventional wet granulation is used, the active ingredient, and excipients are transferred to an appropriate size granulating bowl, and mixed. Water is added to the mixture using an atomizer whilst mixing is in progress, until a granule is formed. The granule is then dried until the desired granule moisture content has been achieved, preferably 2.5% to 3% moisture. The granule is screened/sieved and blended. Magnesium stearate, or other suitable lubricant is added and further blended. The lubricated mixture is compressed into tablets of desired weight and physical specifications by methods known to those skilled in the art.

### EXAMPLE 1

### IMMEDIATE RELEASE (IR) FORMULATION of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate

### Platform Granule for 1 mg and 5 mg Tablets

| Ingredient | % w/w |
|---|---|
| Drug Substance as the tosylate salt | 5.80 |
| Microcrystalline cellulose (Avicel PH101) | 20.00 |
| Lactose (Monohydrate Regular) | 71.20 |
| Kollidon 30 | 3.00 |
| Sterile Water | qs |
| Total | 100.00 |

### Platform Granule for 5 mg to 10 mg Tablets

| Ingredient | % w/w |
|---|---|
| Drug Substance as the tosylate salt | 11.60 |
| Microcrystalline cellulose (Avicel PH101) | 18.73 |
| Lactose (Monohydrate Regular) | 66.67 |
| Kollidon 30 | 3.00 |
| Sterile Water | qs |
| Total | 100.00 |

### For a 2.5 mg Tablet

| Ingredient | % w/w |
|---|---|
| Platform Granule for 1 mg to 5 mg Tablets | 20.00 |
| Lactose (Anhydrous) | 24.88 |
| Microcrystalline cellulose (Avicel PH102) | 49.77 |
| Sodium Starch Glycolate (Glycolis) | 5.00 |
| Magnesium Stearate | 0.35 |
| Total | 100.00 |

### For a 5 mg TABLET

| Ingredient | % w/w |
|---|---|
| Platform Granule for 1 mg to 5 mg Tablets | 40.00 |
| Lactose (Anhydrous) | 18.22 |
| Microcrystalline cellulose (Avicel PH102) | 36.43 |
| Sodium Starch Glycolate (Glycolis) | 5.00 |
| Magnesium Stearate | 0.35 |
| Total | 100.00 |

### For a 7.5 mg TABLET

| Ingredient | % w/w |
|---|---|
| Platform Granule for 5 mg to 10 mg Tablets | 30.00 |
| Lactose (Anhydrous) | 21.55 |
| Microcrystalline cellulose (Avicel PH102) | 43.10 |
| Sodium Starch Glycolate (Glycolis) | 5.00 |
| Magnesium Stearate | 0.35 |
| Total | 100.00 |

### For a 10 mg TABLET

| Ingredient | % w/w |
|---|---|
| Platform Granule for 5 mg to 10 mg Tablets | 40.00 |
| Lactose (Anhydrous) | 18.22 |
| Microcrystalline cellulose (Avicel PH102) | 36.43 |
| Sodium Starch Glycolate (Glycolis) | 5.00 |
| Magnesium Stearate | 0.35 |
| Total | 100.00 |

### Bulk Preparation Method

The components of the appropriate platform granule in each of Examples 1 through 5 were weighed and passed through a 1 mm sieve into a high shear granulator bowl, such as a PMA65 bowl. The ingredients were dry blended for 3 minutes using an impellor speed of 300 rpm. Water for binding was added over a 4 minute using a peristaltic pump delivering water at approximately 600 g/min. During water addition the impellor speed was 300 rpm and the chopper speed was I. After addition of the water the mixture was wet massed for 10 minutes using the same impellor speed and a chopper speed of II.

The wet granules were emptied from the granulating bowl into a fluid bed drier, such as a Glatt 3/5. The granules were dried using an air feed rate of 205 m³/hr and an inlet temperature of 70° C. The granules were dried until the LOD was approximately 1 to 3% w/w. The granule was milled through a 0.094″ comil screen.

The appropriate quantities of platform granule and excipients with the exception of Magnesium Stearate were weighed into a 100 L blending drum and blended using a blender such as a Fordertechnik for 10 mins at 17 rpm. The Magnesium Stearate was weighed and added to the blend and the mixture was blended for a farther 2 minutes at 17 rpm. Using a suitable rotary tablet press, such as a Betapress or equivalent, the blend was compressed into 9.0 mm round tablets, with a target weigh of 300 mg (range 285 mg to 315 mg) and a target thickness of 4.5 mm (range 4.0 mm to 5.0 mm).

A 12% w/w aqueous film coat suspension was prepared by dispersing the required quantity of opadry powder in water, with the aid of a suitable size paddle mixer.

A suitable film coating machine was pre-heated to 40° C. for 15 mins. The tablet cores were added to the film coating machine and rotated at 20 rpm at 40° C. The aqueous film coat suspension was sprayed onto the tablet cores at a rate of approximately 4.5 g/min, until an approximately 3% weight gain was achieved.

### Bulk Preparation Method for Direct Compression IR Tablet

The drug and excipients (except Magnesium Stearate lubricant) are weighed and transferred to a suitable blending drum or blender, such as a Pharma-Tech cube blender. They are then blended together for 15 minutes at 17 rpm.

An excess of Magnesium Stearate is sieved through a 250 micron screen and the required quantity dispensed. The Magnesium Stearate is then added to the blend and blended for a further 1 minute at 17 rpm.

The final blend is then transferred to a suitable rotary tablet press, such as a Killian and compressed into 9.0 mm round tablets, with a target weight of 300 mg (range 285 mg to 315 mg) and a target thickness of 4.5 mm (range 4.0 mm to 5.0 mm)

Tablet weight, thickness and hardness checks are carried out at regular intervals throughout the compression run to ensure the tablets were within specification. A friability test is carried out at the beginning and end of run to ensure the tablets are robust enough for coating

A 12% w/w aqueous film coat suspension is prepared by dispersing the required quantity of opadry powder in water, with the aid of a suitable size paddle mixer.

A suitable coater is preheated to 40° C. for approx 15 minutes and loaded with the tablet cores. The cores are then coated at a speed of 20 rpm using a spray rate of 4.5 g-6.0 g/minute until approximately 3% w/w film coat (based on core weight) is applied.

Tablet weight may be monitored at regular intervals throughout the coating run to determine the film coat end point.

A final sample of coated tablets is suitably taken to determine the mean weight, thickness, hardness and to assess the quality of the coat.

### EXAMPLE 2

### MODIFIED RELEASE (MR) FORMULATION of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate

Matrix Tablets with 30% Polymer
Polymers used herein are Methocel K100 LV or Hypromellose 2208

Quantity Component (mg/tablet) (% w/w):

| 8-(2,6-difluorophenyl)-4-(4-fluoro-2- | 10.44 mg/6.96% w/w |
|---|---|
| methylphenyl)-2-{[2-hydroxy-1- | |
| (hydroxymethyl)ethyl]amino}pyrido[2,3- | |
| d]pyrimidin-7(8H)-one, tosylate | |
| Lactose (anhydrous) | 71.01 mg/47.3% w/w |
| Microcrystalline cellulose (Avicel PH200) | 22.50 mg/15.0% w/w |
| Methocel KL00LV | 45.00 mg/30.00% w/w |
| Magnesium Stearate | 0.75 mg/0.50% w/w |
| Silicon Dioxide (anhydrous) | 0.30 mg/0.20% w/w |
| | 150 mg Total Tablet Weight |
| | (100%) |

### Bulk Preparation Method

First the components were weighed from bulk containers in the following amounts as noted above.

The drug substance, microcrystalline cellulose, lactose anhydrous, hypromellose 2208 and colloidal silicon dioxide were transferred into a blending drum, or suitable blender, such as a Pharma-Tech Cube Blender. The drug substance and excipients were blended together for 5 minutes at 17 RPM. The blended ingredients were sieved through a 0.032 inch screen and then blended for a further 10 minutes at 17 RPM. Magnesium stearate was added to the blend and mixed for 1 minute at 17 RPM.

The blended drug substance and excipients were compressed, using a suitable rotary tablet press, typically a Fette 2090 or equivalent into 7.5 mm round tablets at a target compression weight of 150 mg (range 142 to 158 mg) and a target thickness of 3.0 to 3.5 mm.

In-process controls for tablet weight and hardness were applied at appropriate intervals throughout the compression run and adjustments to the tablet press may be made as necessary.

### EXAMPLE 3

### MODIFIED RELEASE (MR) FORMULATION of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate

Matrix Tablets with 40% Polymer

Polymers are either Methocel K100LV or Hypromellose 2208

Quantity Component (mg/tablet) (% w/w)

| 8-(2,6-difluorophenyl)-4-(4-fluoro-2- | 10.44 mg/6.96% w/w |
|---|---|
| methylphenyl)-2-{[2-hydroxy-1- | |
| (hydroxymethyl)ethyl]amino}pyrido[2,3- | |
| d]pyrimidin-7(8H)-one, tosylate | |
| Lactose (anhydrous) | 39.26 mg/26.17% w/w |
| Microcrystalline cellulose (Avicel PH200) | 39.26 mg/26.17% w/w |
| Methocel K4M | 60.00 mg/40.00% w/w |
| Magnesium Stearate | 0.75 mg/0.50% w/w |
| Silicon Dioxide (anhydrous) | 0.30 mg/0.20% w/w |
| | 150 mg Total Tablet Weight |
| | (100%) |

### Bulk Preparation Method

The components were weighed from bulk containers in the amounts as noted above, and processed as indicated for Example 2, yielding a dosage strength of 7.5 mg/tablet.

### EXAMPLE 4

### MODIFIED RELEASE (MR) FORMULATION of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate

Matrix Tablets with 25% Polymer
Polymers are either Methocel K4MP or Hypromellose 2208

Quantity Component (mg/tablet) (% w/w)

| 8-(2,6-difluorophenyl)-4-(4-fluoro-2- | 10.44 mg/6.96% w/w |
|---|---|
| methylphenyl)-2-{[2-hydroxy-1- | |
| (hydroxymethyl)ethyl]amino}pyrido[2,3- | |
| d]pyrimidin-7(8H)-one tosylate | |
| Lactose (anhydrous) | 50.51 mg/33.67% w/w |
| Microcrystalline cellulose (Avicel PH200) | 50.51 mg/33.67.0% w/w |
| Methocel K4M | 37.50 mg/25.00% w/w |
| Magnesium Stearate | 0.75 mg/0.50% w/w |
| Silicon Dioxide (anhydrous) | 0.30 mg/0.20% w/w |
| | 150 mg Total Tablet Weight |
| | (100%) |

### Bulk Preparation Method

The components were weighed from bulk containers in the amounts as noted above, and processed as indicated for Example 2, yielding a dosage strength of 7.5 mg/tablet.

### EXAMPLE 5

### MODIFIED RELEASE (MR) FORMULATION of 8-(2,6-difluorophenyl)-4-(4-fluoro-2-methylphenyl)-2-([2-hydroxy-1-(hydroxymethyl)ethyl]amino)pyrido[2,3-d]pyrimidin-7(8H)-one, tosylate

Matrix Tablets with 29% Polymer
Polymers are either Methocel K4M or Hypromellose 2208

Quantity Component (mg/tablet) (% w/w)

| 8-(2,6-difluorophenyl)-4-(4-fluoro-2- | 3.5 mg/1.13% w/w |
|---|---|
| methylphenyl)-2-{[2-hydroxy-1- | |
| (hydroxymethyl)ethyl]amino}pyrido[2,3- | |
| d]pyrimidin-7(8H)-one tosylate | |
| Lactose (monohydrate) | 205.1 mg/66.38% w/w |
| Methocel K4M | 90.0 mg/29.13% w/w |
| Magnesium Stearate | 1.5 mg/0.49% w/w |
| Opadry Film Coating | 9.0 mg/2.91% w/w |
| Sterile Water | qs |
| | 309 mg Total Tablet Weight |
| | (100%) |

### Bulk Preparation Method

With the exception of the magnesium stearate and opadry film coating, all materials were weighed into a granulating bowl in the amounts shown in example 5 above. The powder was mixed in a Eurovent granulator for 3 minutes using an impellor at operating at 300 rpm.

The impeller was set to 500 rpm and the chopper was set at 1000 rpm, water was then added to the mixture at 9 g/min using an atomizer set a 1 bar, until an acceptable granule was produced, the granule was wet massed for 5 minutes using the same setting for the impellor and chopper. The wet granule was transferred to a suitable drier, where it was dried at 60° C., until the loss on drying was approximately 3%.

The granule was transferred to a glass turbula jar and the magnesium stearate was added to the jar. The powder was blended for 1 minute at 22 rpm. The blended granule and magnesium stearate were compressed, using a suitable tablet press, into 9.0 mm round tablets at a target compression weight of 300 mg (range 291 to 309 mg) and a target thickness of 4.1 to 4.5 mm.

In-process controls for tablet weight and hardness may be applied at appropriate intervals throughout the compression run and adjustments to the tablet press may be made as necessary.

A 12% w/w aqueous film coat suspension was prepared by dispersing the required quantity of opadry powder in water, with the aid of a suitable size paddle mixer. A suitable film coating machine was pre-heated to 80° C. for 1 hour. The tablet cores were added to the film coating machine and rotated at 20 rpm at 80° C. The aqueous film coat suspension was sprayed onto the tablet cores at a rate of approximately 4.5 g/min, until an approximately 3% weight gain was achieved.

### EXAMPLE 6

### MODIFIED RELEASE (MR) FORMULATION of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate

Matrix Tablets with 27% Polymer
Polymers are either Methocel K4M or Hypromellose 2208

Quantity Component (mg/tablet) (% w/w)

| 8-(2,6-difluorophenyl)-4-(4-fluoro-2- | 10.4 mg/3.37% w/w |
|---|---|
| methylphenyl)-2-{[2-hydroxy-1- | |
| (hydroxymethyl)ethyl]amino}pyrido[2,3- | |
| d]pyrimidin-7(8H)-one tosylate | |
| Lactose (monohydrate) | 154.7 mg/50.06% w/w |
| Microcrystalline Cellulose | 49.5 mg/16.02% w/w |
| Methocel K4M | 84.0 mg/27.18% w/w |
| Magnesium Stearate | 1.5 mg/0.49% w/w |
| Opadry Film Coating | 9.0 mg/2.91% w/w |
| Sterile Water | qs |
| | 309 mg Total Tablet Weight |
| | (100%) |

### Bulk Preparation Method

The components were weighed from bulk containers in the amounts as noted above, and processed as indicated for Example 5, yielding a dosage strength of 7.5 mg/tablet.

In one embodiment of the invention, a composition of the invention can be administered in a combination therapy with one or more additional drugs or prodrugs as may be necessary or desirable.

The term “combination therapy” herein means a treatment regimen wherein the agent provided by the composition of the invention and a second agent are administered individually or together, sequentially or simultaneously, in such a way as to provide a beneficial effect from co-action of these therapeutic agents. Such beneficial effect can include, but is not limited to, pharmacokinetic or pharmacodynamic co-action of the therapeutic agents. Combination therapy can, for example, enable administration of a lower dose of one or both agents than would normally be administered during monotherapy, thus decreasing risk or incidence of adverse effects associated with higher doses. Alternatively, combination therapy can result in increased therapeutic effect at the normal dose of each agent in monotherapy. “Combination therapy” herein is not intended to encompass administration of two or more therapeutic agents as part of separate monotherapy regimens that incidentally and arbitrarily result in sequential or simultaneous treatment.

Compositions of the invention can be especially suited to combination therapies, particularly where the second agent is one that is, or can be, administered once daily. There are significant advantages in patient convenience and compliance where both components of a combination therapy can be administered at the same time and with the same frequency.

When administered simultaneously, the two components of the combination therapy can be administered in separate dosage forms or in co-formulation, i.e., in a single dosage form. When administered sequentially or in separate dosage forms, the second agent can be administered by any suitable route and in any pharmaceutically acceptable dosage form, for example by a route and/or in a dosage form other than the present composition. In a preferred embodiment, both components of the combination therapy are formulated together in a single dosage form.

The exact dosage and frequency of administration depends the severity of the condition being treated, the weight, general physical condition of the particular patient, other medication the individual may be taking as is well known to those skilled in the art and can be more accurately determined by measuring the blood level or concentration of the free base of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one in the patient's blood and/or the patient's response to the particular condition being treated.

Suitably, in a combination for the treatment of rheumatoid arthritis, the water soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, and in particular the tosylate salt may be administered in combination therapy with disease modifying antirheumatic drugs (DMARDs) such as abatacept (Orencia®), entanercept (Enbrel®), infliximab (Remicade®); adalimumab (Humira®), methotrexate (MTX), hydroxychloroquine (Plaquenil®), sulfasalazine (Azulfidine®), leflunomide (Arava®), Anakinra (Kineret®), rituximab (Rituxin®), or various corticosteroids, such as prednisone; NSAID's, COX-2 inhibitors (Vioxx, Celebrex, Bextra®), nonacetylated salicylates (Trilisate or Disalcid®); alone or in combination with each other. Other DMARD's which are used less frequently include but are not limited to azathioprine, cyclosporine, D-penicilliamine, gold salts and minocycline, alone or in combination with the other DMARD's. Recently the class of drugs known as the statins (atorvastatin, fluvastatin, lovastatin, pravastatin, rosuvastatin, and simvastatin) have been suggested for treatment of rheumatoid arthritis. Supportive options for use with these agents, include elemental calcium, vitamin D, hormone replacement therapy, and antiresorptive agents, as well as raloxifene (generally for use with low dose corticosteroid treatment). Other supportive agents for use with the NSAID or COX-2 inhibitors includes the use of a gastroprotective agent such as a proton-pump inhibitor, or an oral prostaglandin analog (Cytotec®).

While recognizing that the frequency, duration and dosage of these DMARDs may vary with the severity of the condition being treated, the weight, general physical condition of the particular patient, and other medication the individual may be taking, the table below is a recommended dosage schedule for maintenance therapy as is well known to those skilled in the art.

| Drug | Usual Dose For Maintenance Therapy |
|---|---|
| Methotrexate | Oral: 7.5-20 mg/week |
| | Injectable: 7.5-20 mg/week |
| Hydroxychloroquine | 200 mg twice daily |
| (Plaquenil) | |
| Sulfasalazine (Azulfidine) | 1,000 mg 2-3 times daily |
| Leflunomide (Arava) | If tolerated, 20 mg/day in a single dose. If |
| | not tolerated, 10 mg/day. |
| Etanercept (Enbrel) | 25 mg SC^{b} twice per week |
| Infliximab (Remicade) with | 3-10 mg/kg IV^{b} every 8 weeks or |
| methotrexate | 3-5 mg/kg IV^{b} every 4 weeks |
| Adalimumab (Humira) | 40 mg every other week or 40 mg every |
| | week SC^{b} |
| Anakinra | 100 mg SC^{b} daily |
| (Kineret) | |
| Corticosteroids | <10 mg daily of prednisone or equivalent |

^{b}SC = subcutaneously; IV = intravenous infusion

While specific synthetic intermediates, such as those described herein, are demonstrated in the schematic (Scheme 1) shown below for the overall synthesis of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one tosylate salt, this schematic is representative of the general process to make compounds of Formula (II) and (III) which may then have the S(O)m-Rg moiety displaced/reacted with an appropriate “X” moiety such as shown below or as described in a number of patent applications such as WO 02/059083, WO 04/073628, U.S. Pat. No. 6,809,199, WO 2006104889, WO 2006/104915, WO 2006/104917 and US 2006217401. Suitable starting materials and intermediates for use in this reaction are well known in the art and may be produced by standard methods, and may also be found in the above noted applications whose disclosures are incorporated by reference herein.

Again, solely as an illustration of the preparation of compounds of the present invention the compounds in these Schemes are shown with an S-methyl group which is deemed representative of the S(O)m-Rg group, as well as specific R1, R1′, R3, Rg, m, s and t moieties, again which are deemed representative of the substituents on the compounds of Formulas (II), (III), and (IV) as more fully described herein.

It may be desirable during the synthesis of the compounds of this invention, such as compounds of Formula (III), to derivatize reactive functional groups in the molecule undergoing reaction so as to avoid unwanted side reactions. Functional groups such as hydroxy, amino, and acid groups are typically protected with suitable groups that can be readily removed when desired. Suitable common protecting groups for use with hydroxyl groups and nitrogen groups are well known in the art and described in many references, for instance, Protecting Groups in Organic Synthesis, Greene et al., John Wiley & Sons, New York, N.Y., (2nd edition, 1991 or the earlier 1981 version). Suitable examples of hydroxyl protecting groups include ether forming groups such as benzyl, and aryl groups such as tert-butoxycarbonyl (Boc), silyl ethers, such as t-butyldimethyl or t-butyldiphenyl, and alkyl ethers, such as methyl connected by an alkyl chain of variable linkage. Amino protecting groups may include benzyl, aryl such as acetyl and trialkylsilyl groups. Carboxylic acid groups are typically protected by conversion to an ester that can easily be hydrolyzed, for example, trichloethyl, tert-butyl, benzyl and the like.

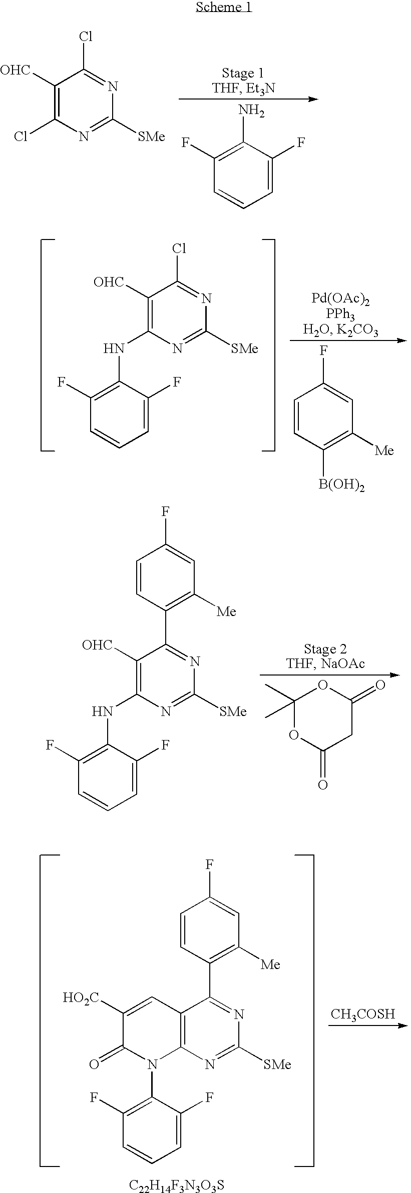

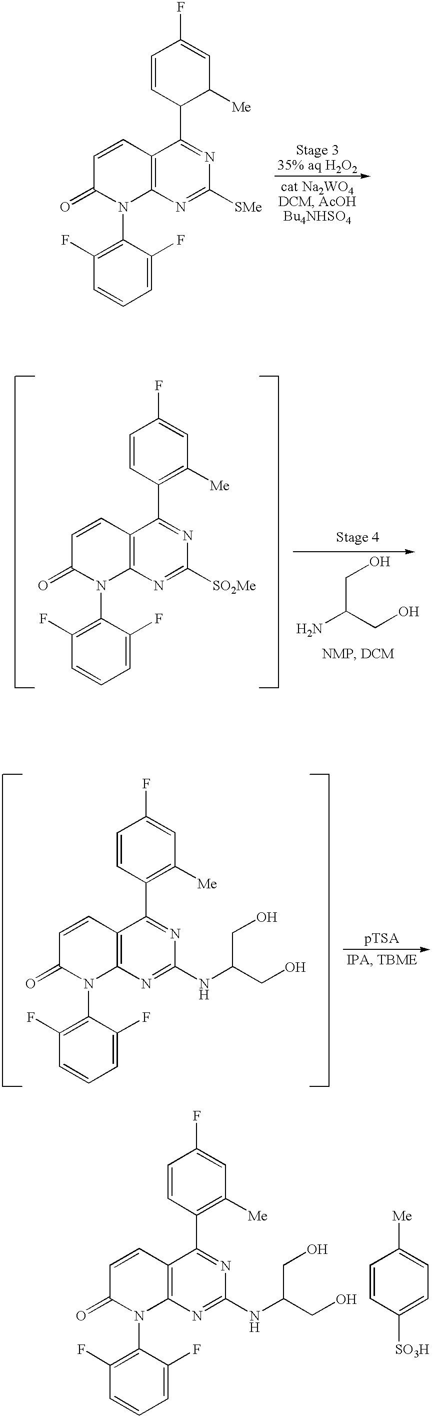

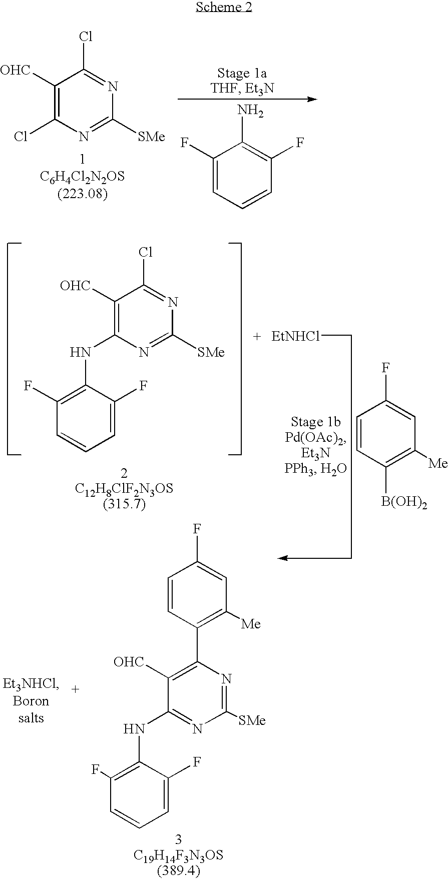

Scheme 2 is a description of a synthetic pathway to make intermediate (3), a compound representative of Formula (IV) and which occurs in a number of the synthetic examples herein.

Another aspect of the present invention are novel compounds of Formula (II) represented by the formula:

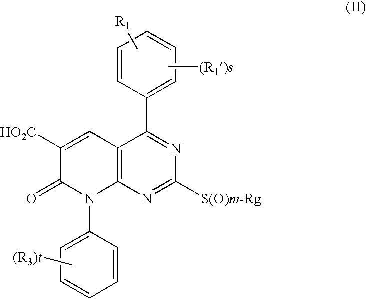

wherein
R₁ is independently selected from hydrogen, C(Z)N(R_{10′})(CR₁₀R₂₀)ᵥR_{b}, C(Z)O(CR₁₀R₂₀)ᵥR_{b}, N(R_{10′})C(Z)(CR₁₀R₂₀)ᵥR_{b}, N(R₁₀)C(Z)N(R_{10′})(CR₁₀R₂₀)ᵥR_{b}, or N(R_{10′})OC(Z)(CR₁₀R₂₀)ᵥR_{b};
R_{1′} is independently selected at each occurrence from hydrogen, halogen, C₁₋₄ alkyl, halo-substituted-C₁₋₄ alkyl, cyano, nitro, (CR₁₀R₂₀)_{v′}NR_{d}R_{d′}, (CR₁₀R₂₀)_{v′}C(O)R₁₂, SR₅, S(O)R₅, S(O)₂R₅, or (CR₁₀R₂₀)_{v′}OR₁₃;
R₃ is independently selected at each occurrence from hydrogen, halogen, C₁₋₄alkyl, or halosubstituted C₁₋₄alkyl;
R₄ and R₁₄ are each independently selected at each occurrence from hydrogen or C₁₋₄ alkyl, or R₄ and R₁₄ together with the nitrogen to which they are attached form a heterocyclic ring of 5 to 7 members, which ring optionally contains an additional heteroatom selected from NR₉;
R₅ is independently selected at each occurrence from hydrogen, C₁₋₄ alkyl, C₂₋₄ alkenyl, C₂₋₄ alkynyl or NR₄R₁₄, excluding the moieties SR₅ being SNR₄R₁₄, S(O)₂R₅ being SO₂H and S(O)R₅ being SOH;
R₉ and R_{9′} are independently selected at each occurrence from hydrogen, or C₁₋₄ alkyl;
R₁₂ is independently selected at each occurrence from hydrogen, C₁₋₄ alkyl, halo-substitutedC₁₋₄ alkyl, C₂₋₄ alkenyl, C₂₋₄ alkynyl, C₃₋₇cycloalkyl, C₃₋₇cycloalkylC₁₋₄ alkyl, C₅₋₇cycloalkenyl, C₅₋₇cycloalkenylC₁₋₄alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R₁₃ is independently selected at each occurrence from hydrogen, C₁₋₄ alkyl, halo-substituted C₁₋₄ alkyl, C₂₋₄ alkenyl, C₂₋₄ alkynyl, C₃₋₇ cycloalkyl, C₃₋₇cycloalkyl C₁₋₄ alkyl, C₅₋₇ cycloalkenyl, C₅₋₇cycloalkenyl C₁₋₄ alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R_{d} and R_{d′} are each independently selected at each occurrence from hydrogen, C₁₋₄ alkyl, C₃₋₆cycloalkyl, C₃₋₆cycloalkyl C₁₋₄alkyl moiety, and wherein each of these moieties, excluding hydrogen, may be optionally substituted; or R_{d} and R_{d′} together with the nitrogen which they are attached form an optionally substituted heterocyclic ring of 5 to 6 members, which ring optionally contains an additional heteroatom selected from oxygen, sulfur or NR_{9′};
R_{b} is hydrogen, C₁₋₁₀ alkyl, C₃₋₇ cycloalkyl, C₃₋₇ cycloalkyl C₁₋₁₀ alkyl, aryl, arylC₁₋₁₀alkyl, heteroaryl, heteroarylC₁₋₁₀ alkyl, heterocyclic, or heterocyclylC₁₋₁₀ alkyl moiety, which moieties, excluding hydrogen, may all be optionally substituted;
R_{g} is C₁₋₁₀ alkyl, or aryl;
m is 0 or an integer having a value of 1, or 2;
s is an integer having a value of 1, 2, 3 or 4; and
t is an integer having a value of 1, 2, 3 or 4.
v is 0 or an integer having a value of 1 or 2;
v′ is independently selected at each occurrence from 0 or an integer having a value of 1 or 2;
Z is independently selected from oxygen or sulfur;
R₁₀ and R₂₀ are independently selected at each occurrence from hydrogen or C₁₋₄alkyl; and
R_{10′} is independently selected at each occurrence from hydrogen or C₁₋₄alkyl.
Suitably, R_{1′} is independently selected at each occurrence from hydrogen, halogen, C₁₋₄ alkyl, halo-substituted-C₁₋₄ alkyl, cyano, nitro, (CR₁₀R₂₀)_{v′}NR_{d}R_{d′}, (CR₁₀R₂₀)_{v′}C(O)R₁₂, SR₅, S(O)R₅, S(O)₂R₅, or (CR₁₀R₂₀)_{v′}OR₁₃.

In one embodiment, R_{1′} is independently selected from hydrogen, halogen, C₁₋₄ alkyl, or halo-substituted-C₁₋₄ alkyl. The halogen is preferably selected from fluorine or chlorine, the C₁₋₄ alkyl is methyl, and the halo-substituted-C₁₋₄ alkyl is CF₃. In another embodiment R_{1′} is independently selected from hydrogen, halogen, or C₁₋₄alkyl. Preferably the halogen is selected from fluorine or chlorine, and the C₁₋₄ alkyl is methyl. In one embodiment of invention, the phenyl ring is substituted independently 1 or 2 times by fluorine, or methyl.

Suitably, s is an integer having a value of 1, 2, 3 or 4. Preferably when s is 1, R₁ is hydrogen.

Suitably, R₁ is independently selected from hydrogen, C(Z)N(R_{10′})(CR₁₀R₂₀)ᵥR_{b}, C(Z)O(CR₁₀R₂₀)ᵥR_{b}, N(R_{10′})C(Z)(CR₁₀R₂₀)ᵥR_{b}, N(R_{10′})C(Z)N(R_{10′})(CR₁₀R₂₀)ᵥR_{b}, or N(R_{10′})OC(Z)(CR₁₀R₂₀)ᵥR_{b}.

In one embodiment, R₁ is C(Z)O(CR₁₀R₂₀)ᵥR_{b}, R_{b} is C₁₋₁₀ alkyl, Z is oxygen and v is 0. Preferably, R_{b} is methyl.

In one embodiment R₁ is C(Z)O(CR₁₀R₂₀)ᵥR_{b}, R_{b} is methyl, Z is oxygen, v is 0, and R_{1′} is methyl. Preferably, R₁ is in the 5-position and R_{1′} is in the 2-position.

The phenyl ring when substituted by R₁ is preferably in the 2, 4, or 6-position, or di-substituted in the 2,4-position, such as 2-fluoro, 4-fluoro, 2,4-difluoro, or 2-methyl-4-fluoro; or tri-substituted in the 2,4,6-position such as 2,4,6-trifluoro.

The phenyl ring when substituted by R₁ is preferably in the 2-position, if R_{1′} is hydrogen, and in the 5-position when R_{1′} is other than hydrogen. Preferably when the ring is disubstituted by both R₁ and R_{1′} it is substituted in the 2,5-position. More preferably R_{1′} is in the 2-position, and R₁ is in the 5-position.

Suitably, R₄ and R₁₄ are each independently selected at each occurrence from hydrogen or C₁₋₄ alkyl, or R₄ and R₁₄ together with the nitrogen to which they are attached form a heterocyclic ring of 5 to 7 members, which ring optionally contains an additional heteroatom selected from NR_{9′}.

Suitably, R₅ is independently selected at each occurrence from hydrogen, C₁₋₄ alkyl, C₂₋₄ alkenyl, C₂₋₄ alkynyl or NR₄R₄, excluding the moieties SR₅ being SNR₄R₁₄, S(O)₂R₅ being SO₂H and S(O)R₅ being SOH.

Suitably, R₉ and R_{9′} are independently selected at each occurrence from hydrogen, or C₁₋₄ alkyl.

Suitably, R₁₂ is independently selected at each occurrence from hydrogen, C₁₋₄ alkyl, halo-substituted C₁₋₄ alkyl, C₂₋₄ alkenyl, C₂₋₄ alkynyl, C₃₋₇ cycloalkyl, C₃₋₇cycloalkylC₁₋₄ alkyl, C₅₋₇ cycloalkenyl, C₅₋₇cycloalkenyl C₁₋₄ alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted.

Suitably, R₁₃ is independently selected at each occurrence from hydrogen, C₁₋₄ alkyl, halo-substituted C₁₋₄ alkyl, C₂₋₄ alkenyl, C₂₋₄ alkynyl, C₃₋₇ cycloalkyl, C₃₋₇cycloalkylC₁₋₄ alkyl, C₅₋₇ cycloalkenyl, C₅₋₇cycloalkenyl C₁₋₄ alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted.

Suitably, R_{d} and R_{d′} are each independently selected at each occurrence from hydrogen, C₁₋₄ alkyl, C₃₋₆ cycloalkyl, C₃₋₆ cycloalkylC₁₋₄alkyl moiety, and wherein each of these moieties, excluding hydrogen, may be optionally substituted; or R_{d} and R_{d′} together with the nitrogen which they are attached form an optionally substituted heterocyclic ring of 5 to 6 members, which ring optionally contains an additional heteroatom selected from oxygen, sulfur or NR_{9′}.

Suitably, R_{b} is hydrogen, C₁₋₁₀ alkyl, C₃₋₇ cycloalkyl, C₃₋₇ cycloalkyl C₁₋₁₀ alkyl aryl, arylC₁₋₁₀alkyl, heteroaryl, heteroarylC₁₋₁₀ alkyl, heterocyclic, or heterocyclylC₁₋₁₀ alkyl moiety, which moieties, excluding hydrogen, may all be optionally substituted.

Suitably, R_{g} is C₁₋₁₀ alkyl, or an aryl. In one embodiment of the invention R_{g} is C₁₋₄ alkyl, preferably methyl or propyl, more preferably methyl.

Suitably, m is 0 or is an integer having a value of 1 or 2. In one embodiment of the invention m is 0. In another embodiment of the invention m is 0, and R_{g} is methyl or propyl, preferably methyl.

Suitably, s is an integer having a value of 1, 2, 3 or 4.

Suitably, t is an integer having a value of 1, 2, 3 or 4.

Suitably, v is 0 or an integer having a value of 1 or 2.

Suitably, v′ is independently selected at each occurrence from 0 or an integer having a value of 1 or 2.

Suitably, Z is independently selected from oxygen or sulfur.

Suitably, R₁₀ and R₂₀ are independently selected at each occurrence from hydrogen or C₁₋₄ alkyl.

Suitably, R_{10′} is independently selected at each occurrence from hydrogen or C₁₋₄alkyl.

Suitably, R₃ is independently selected from hydrogen, halogen, C₁₋₄alkyl, or halosubstituted C₁₋₄alkyl. Preferably the halogen is fluorine or chlorine, the C₁₋₄ alkyl is methyl, and the halo-substituted-C₁₋₄ alkyl is CF₃. More preferably, the phenyl ring is substituted by R₃ independently at each occurrence from halogen, or C₁₋₄alkyl, e.g. fluorine or methyl. In one embodiment of invention, the phenyl ring is substituted independently 1, 2 or 3 times by fluorine, e.g. t is 1, 2, or 3.

Preferably, the phenyl ring when substituted by R₃ is in the 2, 4, or 6-position, or di-substituted in the 2,4-position, such as 2-fluoro, 4-fluoro, 2,4-difluoro, or 2,6-difluoro, 2-methyl-4-fluoro; or tri-substituted in the 2,4,6-position, such as 2,4,6-trifluoro.

In one embodiment of the invention when t is 1, R₃ is hydrogen.

A compound of Formula (III) is represented by the structure:

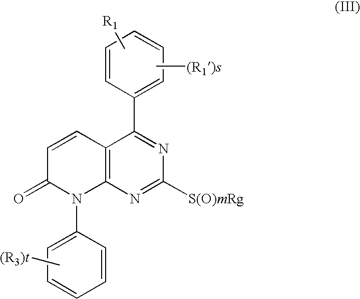

wherein
R₁ is independently selected from hydrogen, C(Z)N(R_{10′})(CR₁₀R₂₀)ᵥR_{b}, C(Z)O(CR₁₀R₂₀)ᵥR_{b}, N(R₁₀)C(Z)(CR₁₀R₂₀)ᵥR_{b}, N(R₁₀)C(Z)N(R₁₀)(CR₁₀R₂₀)ᵥR_{b}, or N(R₁₀)OC(Z)(CR₁₀R₂₀)ᵥR_{b};
R_{1′} is independently selected at each occurrence from hydrogen, halogen, C₁₋₄ alkyl, halo-substituted-C₁₋₄ alkyl, cyano, nitro, (CR₁₀R₂₀)_{v′}NR_{d}R_{d′}, (CR₁₀R₂₀)_{v′}C(O)R₁₂, SR₅, S(O)R₅, S(O)₂R₅, or (CR₁₀R₂₀)_{v′}OR₁₃;
R₃ is independently selected at each occurrence from hydrogen, halogen, C₁₋₄alkyl, or halosubstituted C₁₋₄alkyl;
R₄ and R₁₄ are each independently selected at each occurrence from hydrogen or C₁₋₄ alkyl, or R₄ and R₁₄ together with the nitrogen to which they are attached form a heterocyclic ring of 5 to 7 members, which ring optionally contains an additional heteroatom selected from NR₉;
R₅ is independently selected at each occurrence from hydrogen, C₁₋₄ alkyl, C₂₋₄ alkenyl, C₂₋₄ alkynyl or NR₄R₁₄, excluding the moieties SR₅ being SNR₄R₁₄, S(O)₂R₅ being SO₂H and S(O)R₅ being SOH;
R₉ and R₁₉ are independently selected at each occurrence from hydrogen, or C₁₋₄ alkyl;
R₁₂ is independently selected at each occurrence from hydrogen, C₁₋₄ alkyl, halo-substitutedC₁₋₄ alkyl, C₂₋₄ alkenyl, C₂₋₄ alkynyl, C₃₋₇cycloalkyl, C₃₋₇cycloalkylC₁₋₄ alkyl, C₅₋₇cycloalkenyl, C₅₋₇cycloalkenylC₁₋₄alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R₁₃ is independently selected at each occurrence from hydrogen, C₁₋₄ alkyl, halo-substituted C₁₋₄ alkyl, C₂₋₄ alkenyl, C₂₋₄ alkynyl, C₃₋₇ cycloalkyl, C₃₋₇cycloalkyl C₁₋₄ alkyl, C₅₋₇ cycloalkenyl, C₅₋₇cycloalkenyl C₁₋₄ alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R_{d} and R_{d′} are each independently selected at each occurrence from hydrogen, C₁₋₄ alkyl, C₃₋₆cycloalkyl, C₃₋₆cycloalkyl C₁₋₄alkyl moiety, and wherein each of these moieties, excluding hydrogen, may be optionally substituted; or R_{d} and R_{d′} together with the nitrogen which they are attached form an optionally substituted heterocyclic ring of 5 to 6 members, which ring optionally contains an additional heteroatom selected from oxygen, sulfur or NR_{9′};
R_{b} is hydrogen, C₁₋₁₀ alkyl, C₃₋₇ cycloalkyl, C₃₋₇ cycloalkyl C₁₋₁₀ alkyl, aryl, arylC₁₋₁₀ alkyl, heteroaryl, heteroarylC₁₋₁₀ alkyl, heterocyclic, or heterocyclylC₁₋₁₀ alkyl moiety, which moieties, excluding hydrogen, may all be optionally substituted;
R_{g} is C₁₋₁₀ alkyl, or aryl;
m is 0 or an integer having a value of 1, or 2;
s is an integer having a value of 1, 2, 3 or 4; and
t is an integer having a value of 1, 2, 3 or 4.
v is 0 or an integer having a value of 1 or 2;
v′ is independently selected at each occurrence from 0 or an integer having a value of 1 or 2;
Z is independently selected from oxygen or sulfur;
R₁₀ and R₂₀ are independently selected at each occurrence from hydrogen or C₁₋₄ alkyl; and
R_{10′} is independently selected at each occurrence from hydrogen or C₁₋₄alkyl.

A compound of Formula IV is represented by the structure:

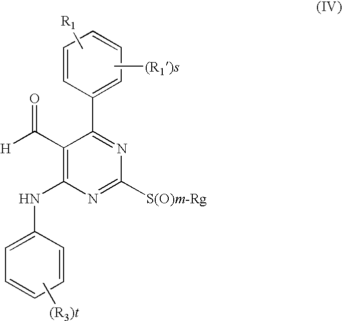

wherein R₁, R_{1′}, R₃, s, and t, etc. are as described above for Formula (II), Rg is C₁₋₁₀alkyl, or aryl, and m is 0, 1 or 2.

Suitably, Rg is a C₁₋₁₀alkyl, or aryl, preferably Rg is C₁₋₄alkyl, more preferably methyl or propyl. In one embodiment of the invention, m is 0 and Rg is methyl or propyl, preferably methyl.

While Scheme I demonstrates the decarboxylation step using a thioacetate derivative, any thioacid derivative could be used, e.g. thioacetic, thiobenzoic and thiopropionic, or a salt thereof. Use of a salt of a suitable thioacid, such as potassium, sodium, calcium, magnesium, cesium or lithium salts are within the context of this invention. The pKa range of thioacids is <0. Therefore, it is believed that an important feature of the thioacid used is the nucleophilicity of its corresponding thiocarboxylate derivative. Use of any suitable thioacid to achieve decarboxylation on a pyridine ring or a bicyclo pyridinepyrimidine ring is believed to be a novel feature of this process.

This reaction may comprise use of an organic solvent, optionally in combination with water. Suitably the organic solvent is one which has a boiling point which can go up to 110° C., or at reflux of the solvent. Solvents include but are not limited to THF, ethyl acetate, DIPEA, pyridine, toluene, DMF, n-methylpyrrolidine, methylene chloride, dioxane, or acetonitrile. In one embodiment of the invention the organic solvent is THF or toluene.

While temperature is generally not an issue for this reaction, it typically is at a temperature slightly above room temperature, suitably around 30° C. At temperatures below 30° C. the reaction proceeds at a slower pace. In one embodiment the reaction is run from about 20 to about 50° C.

In one embodiment of the invention the thioacid is potassium thioacetate, sodium thioacetate, calcium thioacetate, magnesium thioacetate, cesium thioacetate or lithium thioacetate.

Therefore a novel process of this invention, as shown in Scheme 3 below, is the decarboxylation of a compound of Formula (II) as described above using a thioacid derivative to yield a compound of Formula (III), wherein s, t, m, Rg, R₁, R_{1′} and R₃ are as described for Formula (II) above:

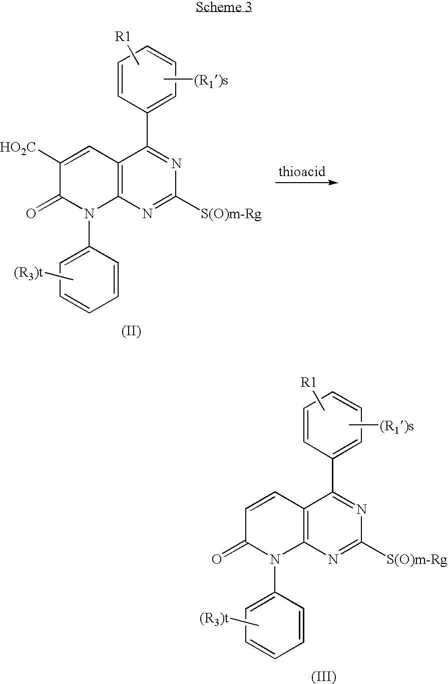

In one embodiment of this invention suitably, m is 0. In another embodiment of the invention m is 0, and Rg is a C₁₋₁₀alkyl, preferably methyl or propyl, more preferably methyl.

Another aspect of the invention is the novel process of ring cyclization of a compound of Formula (IV) to a compound of Formula (II) by use of meldrums acid, or a suitable equivalent, such as malonic acid (uncyclized) in an organic solvent with a suitable base. Use of meldrums acid on a benzene substrate has previously been used to form a bicyclic system, such as shown in Suzuki, M., et al., Chem. Pharm. Bull., 49 (1), 29 (2001); Suzuki, M. et al., Heterocycles, 53 (11) 2471 (2000); or Kaneko, T., et al., Jpn. Kokai Tokkyo Koho, 10245374, 14 Sep. 1998, Heisei. However, formation of a bicyclo system of Formula (II) using a pyrimidine substrate of Formula (IV) is believed novel. Use of malonic acid is also believed to be similarly novel. Blano, M. et al., Heterocycles, 36 (6) 1387 (1993); Hayes, R. et al., Tetrahydron Lett., 23 (15), 1613 (1982); and Lippmann, E. et al., Zeitschrift far Chemie, 19 (11), 422 (1979).

Suitable bases for use herein include both inorganic and organic bases.

Suitable organic bases for use herein include but are not limited to 2,3-lutidine, 2,4,6-collidine, 2,5-dimethylpiperazine, 2,6-dimethylpiperidine, 2,6-di-tert-butylpyridine, 2,6-lutidine, 2-methylpiperidine, 4-methylbenzylamine, 4-methylcyclohexylamine, 4-methylmorpholine, 4-phenylmorpholine, benzylamine, butylamine, cyclohexylamine, cyclopentylamine, DABCO, DBN, DBU, dicyclohexylamine, diethylamine, dihexylamine, diisopropylamine, DIPEA, diphenylamine, dipropylamine, di-sec-butylamine, DMAP, ethylamine, isobutylamine, isopentylamine, isopropylamine, isoquinoline, morpholine, N-ethylpiperidine, N-methylbutylamine, N-methylpiperazine, N-methylpiperidine, piperazine, piperidine, pyridine, pyrrolidine, quinoline, sec-butylamine, tert-butylamine, tetramethylpyrazine, tributylamine, triethylamine, tripropylamine

In one embodiment of the invention the organic base is 2,4,6-collidine, DIPEA, DBN, dihexylamine, diethylamine, di-sec-butylamine, dimethylamine, isopropylamine, dipropylamine, isoquinoline, 2,6,-lutidine, N-methylpiperidine, 2,6-dimethylpiperidine, pyridine, pyrrolidine, or triethylamine.

Suitable inorganic bases for use herein include but are not limited to ammonia, barium carbonate, barium hydroxide, calcium carbonate, calcium hydroxide, cesium carbonate, cesium hydroxide, lithium carbonate, lithium hydroxide, magnesium carbonate, magnesium hydroxide, potassium acetate, potassium amide, potassium carbonate, potassium dihydrogen phosphate, potassium ethoxide, potassium hydride, potassium hydrogen carbonate, potassium hydrogen phosphate, potassium hydroxide, potassium methoxide, potassium phosphate, potassium-t-butoxide, rubidium carbonate, sodium acetate, sodium amide, sodium carbonate, or sodium methoxide

In one embodiment of the invention the inorganic base is cesium hydroxide, cesium carbonate, and acetate salts, such as sodium acetate, cesium acetate, magnesium acetate, calcium acetate, or potassium acetate.

In another embodiment the base is sodium, potassium or cesium acetate, 2,6-dimethyl piperidine, DIPEA, 2,4,6-collidine, or dihexylamine.

As noted in the working examples, the bases may be divided into liquid or solid bases instead of inorganic/organic. Under alternative conditions it is expected that the solid bases as illustrated in the examples would work, e.g. using lipophilic solvent, or changes in the reaction temperature.

Suitably, the reaction is above room temperature, e.g. 40 to 70° C. or higher. In one embodiment the reaction is run at about 55° C.+/−10° C.

Suitably the organic solvent is one which has a boiling point which can go up to 110° C., or at reflux of the solvent. Suitable organic solvents for use herein include but are not limited to trifluorotoluene, triethyleneglycol dimethyl ether, triethylene glycol, triethylamine, trichloroethane, toluene, tetrahydrofuran, tetraethylene glycol, tert-butylmethylether, quinolone, pyridine, propyl acetate, propionic acid, propanenitrile, propan-2-ol, propan-1-ol, piperidine, pentane, pentan-3-one, pentan-2-ol, nonane, N-methylformamide, N-methylacetamide, nitro-methane, nitrobenzene, n-butyl acetate, N,N-dimethylformamide, N,N-dimethylacetamide, methyl isobutyl ketone, methyl acetate, methanol, isopropyl acetate, HMPT, hexane, heptane, formamide, fluorobenzene, ethyl benzene, ethyl acetate, ethoxybenzene, ethanol, DMPU, DMEU, dipropylether, diphenylether, dimethylsulfoxide, diisopropylether, diethylether, diethyleneglycol dimethylether, diethylene glycol, diethylcarbonate, dichloromethane, dibutylether, cyclohexanone, cyclohexanol, cyclohexane, cis-decaline, chloroform, chlorobenzene, butan-2-one, butan-2-ol, butan-1-ol, benzyl alcohol, benzonitrile, anisole, acetophenone, acetonitrile, acetone, acetic acid, 4-methyl-1,3-dioxol-2-one, 3-pentanol, 3-methylbutan-1-ol, 3-methyl-2-butanone, 3,3-dimethyl-2-butanone, 2-pentanone, 2-methyl-2-propanol, 2-methyl-2-butanol, 2-methyl-1-propanol, 2-methoxyethanol, 2-aminoethanol, 2,6-dimethyl-3-heptanone, 2,4-dimethyl-3-pentanone, 2,2,4-trimethyl pentane, 1-pentanol, 1-methyl-2-pyrrolidinone, 1,4-dioxane, 1,4-dimethylbenzene, 1,3,5-trimethylbenzene, 1,2-ethanediol, 1,2-dimethoxyethane, 1,2-dichloroethane, 1,2-dichlorobenzene, 1,1,3,3-tetramethylurea, 1,1,1-trichloroethane, 2-methyl-tetrahydrofuran, all optionally in combination with water where applicable.

In one embodiment of the invention suitable solvents include THF, DIPEA, pyridine, toluene, DMF, n-methylpyrrolidine, methylene chloride, dioxane, or acetonitrile. It is noted that in some instance the organic base may also be used as the solvent, such as in DIPEA, or pyridine. In another embodiment of the invention, the organic solvent is THF or toluene.

### Method of Treatment Study

A study following an open-label, 4-way, randomized crossover design was conducted in healthy male and female human subjects ranging from 18 to 55 years of age. The subjects received each of the four treatments during the course of the study, at a single center. A total of 26 subjects were enrolled. The subjects were fasted overnight and then given a 7.5 mg oral dose of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one. In the case of the IR formulation, which is provided as a 7.5 mg, composed of a 2.5 mg and 5 mg tablet, given in the morning; in the case of the MR formulations of Examples 2 to 4 herein, a single 7.5 mg tablet was given in the morning. Serial blood samples were taken over a 48-hour period for Pharmacokinetic assessment. Adverse events were recorded during the same time period.

Plasma concentrations were quantitated by an HPLC-MS/MS method, as well as validated. All runs should meet bioanalytical acceptance criteria for calibration standards and quality control.

PK parameters for 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one tosylate were estimated by standard non-compartmental methods. Individual plasma concentration data and the actual time-points of blood sampling from each subject were used in the analysis.

Pharmacokinetic parameters include area under the curve (AUC), maximum observed plasma concentration (Cmax), time of Cmax (Tmax), elimination half-life (T1/2), and the plasma concentration 24 hours after dosing (C24).

Optionally, an in vitro/in vivo correlation for each of the MR formulations can be determined by evaluating a linear relationship of in vivo absorption as a function of in vitro dissolution.

This study should determine various parameters such as Tmax, Cmax, Cmin, AUC_{0-infinity} which may be used herein.

Cmax is well understood in the art as an abbreviation for the maximum drug concentration in serum or plasma of a test subject. In vivo testing protocols can be designed in a number of ways. By measuring the Cmax for a population to which the test composition has been administered and comparing it with the Cmax for the same population to which the control has also been administered, the test composition can be evaluated.

AUC is a determination of the area under the curve (AUC) plotting the serum or plasma concentration of drug along the ordinate (Y-axis) against time along the abscissa (X-axis). Generally, the values for AUC represent a number of values taken from all the subjects in a patient test population and are, therefore, mean values averaged over the entire test population. By measuring the AUC for a population to which the test composition has been administered and comparing it with the AUC for the same population to which the control has been administered, the test composition can be evaluated. Alternatively, the AUC test/AUC control ratio may be determined for each subject, then averaged. AUC's are well understood frequently used tools in the pharmaceutical arts and have been extensively described, for example in “Pharmacokinetics Processes and Mathematics”, Peter E. Welling, ACS Monograph 185; 1986.

Thus, a composition is within the scope of the invention if it effects in vivo either a Cmax or an AUC that is at least 0.80 to 1.25 times of the immediate release formulation (as the comparator) comprising an equivalent quantity of drug and excipients, but without polymer.

Cmax and AUC can be determined in humans or a suitable animal model, such as dogs. Abbreviations: AUC₀₋₂₄=area under the concentration-time curve for 0-24 hours; AUC_{0-infinity}=area under the concentration-time curve for 0-infinity; Cₐᵥ=Calculation of area under the curve over 24 hours (AUC₀₋₂₄) divided by 24 hours; Cₘₐₓ=maximal concentration in plasma; t_{1/2}=half-life; t max-time of maximal concentration in plasma. Coefficient of variation” as used here has its standard meaning, i.e., the ratio of the standard deviation to the mean value for Cmax or AUC.

While it is possible to use a computer simulation model entitled Gastro-Plus (Simulations Plus, Inc.), for calculation of PK parameters for the MR tablets of Examples 2 to 4 herein, human data is available. The above method of treatment study provided the following PK parameters (mean +/−standard deviation) for the IR tablet and for the MR tablets of Examples 2 to 4: AUC_{0-infin} (ng h/ml) 86.8 (IR); 82.9; 75.7; and 62.6, respectively; Cₘₐₓ (ng/ml) 37.5 (IR); 16.6, 10.5; 5.59, respectively; Tₘₐₓ (h). 642 (IR); 3.13; 3.41; and 3.25, respectively.

The MR dosage form of Example 2, was tested in-vitro and provides an in-vitro dissolution rate when measured by the USP I, Basket method (USP I, chapter <711>) at 150 rpm in 500 ml of 0.01M Hydrochloric Acid at 37° C., less than or equal to 20% of 8-(2,6-difluorophenyl)-4-(4-fluoro-2-methylphenyl)-2-{[2-hydroxy-1-(hydroxymethyl)-ethyl]-amino}pyrido[2,3-d]pyrimidin-7(8H)-one, tosylate released after 1 hour, from 26 to 56% of 8-(2,6-difluorophenyl)-4-(4-fluoro-2-methylphenyl)-2-{[2-hydroxy-1-(hydroxymethyl)-ethyl]-amino}pyrido[2,3-d]pyrimidin-7(8H)-one tosylate released after 1.5 hours and greater than 80% of 8-(2,6-difluorophenyl)-4-(4-fluoro-2-methylphenyl)-2-{[2-hydroxy-1-(hydroxymethyl)ethyl]-amino}pyrido[2,3-d]pyrimidin-7(8H)-one, tosylate released after 4 hours.

The MR dosage form of Example 2 was tested in vitro, and provides an in-vitro dissolution rate when measured by the USP 3 Reciprocating Cylinder (USPIII, chapter <711>) method at dip rates between 3 and 10 dips per minute in 250 ml of aqueous buffer (pH between 1.6 and 6.5) at 37° C., less than or equal to 40% of 8-(2,6-difluorophenyl)-4-(4-fluoro-2-methylphenyl)-2-{[2-hydroxy-1-(hydroxymethyl)-ethyl]-amino}pyrido[2,3-d]pyrimidin-7(8H)-one, tosylate released after 1 hour, from 43 to 63% of 8-(2,6-difluorophenyl)-4-(4-fluoro-2-methylphenyl)-2-{[2-hydroxy-1-(hydroxymethyl)ethyl]-amino}pyrido[2,3-d]-pyrimidin-7(8H)-one tosylate released after 2 hours and greater than 80% of 8-(2,6-difluorophenyl)-4-(4-fluoro-2-methylphenyl)-2-{[2-hydroxy-1-(hydroxymethyl)-ethyl]-amino}pyrido[2,3-d]pyrimidin-7(8H)-one tosylate released after 4 hours.

The MR dosage form of Example 3, was tested in-vitro and provides an in-vitro dissolution rate, when measured by the USP I Basket (USP I, chapter <711>) method at 150 rpm in 500 ml of 0.01M Hydrochloric Acid, less than or equal to 20% of 8-(2,6-difluorophenyl)-4-(4-fluoro-2-methylphenyl)-2-{[2-hydroxy-1-(hydroxymethyl)ethyl]-amino}pyrido[2,3-d]pyrimidin-7(8H)-one tosylate released after 1 hour, from 36 to 66% of 8-(2,6-difluorophenyl)-4-(4-fluoro-2-methylphenyl)-2-{[2-hydroxy-1-(hydroxymethyl)-ethyl]-amino}pyrido[2,3-d]pyrimidin-7(8H)-one tosylate released after 2.5 hours and greater than 80% of 8-(2,6-difluorophenyl)-4-(4-fluoro-2-methylphenyl)-2-{[2-hydroxy-1-(hydroxymethyl)ethyl]-amino}pyrido[2,3-d]pyrimidin-7(8H)-one tosylate released after 7 hours.

The MR dosage form of Example 3, was tested in-vitro and provides an in-vitro dissolution rate, when measured by the USP 3 Reciprocating Cylinder (USPIII, chapter <711>) method at dip rates between 3 and 10 dips per minute in 250 ml of aqueous buffer (pH between 1.6 and 6.5) at 37° C., less than or equal to 30% of 8-(2,6-difluorophenyl)-4 (4-fluoro-2-methylphenyl)-2-{[2-hydroxy-1-(hydroxymethyl)ethyl]-amino}pyrido[2,3-d]pyrimidin-7(8H)-one tosylate released after 1 hour, from 40 to 60% of 8-(2,6-difluorophenyl)-4-(4-fluoro-2-methylphenyl)-2-{[2-hydroxy-1-(hydroxymethyl)ethyl]-amino}pyrido[2,3-d]-pyrimidin-7(8H)-one tosylate released after 3 hours and greater than 80% of 8-(2,6-difluorophenyl)-4-(4-fluoro-2-methylphenyl)-2-{[2-hydroxy-1-(hydroxymethyl)-ethyl]-amino}pyrido[2,3-d]pyrimidin-7(8H)-one tosylate released after 8 hours.

The MR dosage form of Example 4, was tested in-vitro and provides an in-vitro dissolution rate, when measured by the USP I Basket (USP I, chapter <711>) method at 150 rpm in 500 ml of 0.01M Hydrochloric Acid, less than or equal to 20% of 8-(2,6-difluorophenyl)-4-(4-fluoro-2-methylphenyl)-2-{[2-hydroxy-1-(hydroxymethyl)ethyl]-amino}pyrido[2,3-d]-pyrimidin-7(8H)-one tosylate released after 1 hour, from 31 to 61% of 8-(2,6-difluorophenyl)-4-(4-fluoro-2-methylphenyl)-2-{[2-hydroxy-1-(hydroxymethyl)-ethyl]-amino}pyrido[2,3-d]pyrimidin-7(8H)-one tosylate released after 4 hours and greater than 80% of 8-(2,6-difluorophenyl)-4-(4-fluoro-2-methylphenyl)-2-{[2-hydroxy-1-(hydroxymethyl)ethyl]-amino}pyrido[2,3-d]pyrimidin-7(8H)-one tosylate released after 7 hours.

The MR dosage form of Example 4, was tested in-vitro and provides an in-vitro dissolution rate, when measured by the USP 3 Reciprocating Cylinder (USPIII, chapter <711>) method at dip rates between 3 and 10 dips per minute in 250 ml of aqueous buffer (pH between 1.6 and 6.5) at 37° C., less than or equal to 30% (of 8-(2,6-difluorophenyl)-4-(4-fluoro-2-methylphenyl)-2-{[2-hydroxy-1-(hydroxymethyl)ethyl]-amino}pyrido[2,3-d]pyrimidin-7(8H)-one tosylate released after 2 hours, from 42 to 62% of 8-(2,6-difluorophenyl)-4-(4-fluoro-2-methylphenyl)-2-{[2-hydroxy-1-(hydroxymethyl)ethyl]-amino}pyrido[2,3-d]-pyrimidin-7(8H)-one tosylate released after 6 hours and greater than 80% of 8-(2,6-difluorophenyl)-4-(4-fluoro-2-methylphenyl)-2-{[2-hydroxy-1-(hydroxymethyl)-ethyl]-amino}pyrido[2,3-d]pyrimidin-7(8H)-one tosylate released after 16 hours.

The MR dosage form of Example 5, was tested in-vitro and provides an in-vitro dissolution rate, when measured by the USP Basket (USP I, chapter <711>) method at 150 rpm in 500 ml of 0.05M Phosphate Buffer at pH 6.0, less than or equal to 20% of 8-(2,6-difluorophenyl)-4-(4-fluoro-2-methylphenyl)-2-{[2-hydroxy-1-(hydroxymethyl)ethyl]-amino}pyrido[2,3-d]-pyrimidin-7(8H)-one tosylate released after 1 hour, from 24 to 54% of 8-(2,6-difluorophenyl)-4-(4-fluoro-2-methylphenyl)-2-{[2-hydroxy-1-(hydroxymethyl)-ethyl]-amino}pyrido[2,3-d]pyrimidin-7(8H)-one tosylate released after 4 hours and greater than 80% of 8-(2,6-difluorophenyl)-4-(4-fluoro-2-methylphenyl)-2-{[2-hydroxy-1-(hydroxymethyl)ethyl]-amino}pyrido[2,3-d]pyrimidin-7(8H)-one tosylate released after 13 hours.

The MR dosage form of Example 6, was tested in-vitro and provides an in-vitro dissolution rate, when measured by the USP Basket (USP I, chapter <711>) method at 150 rpm in 500 ml of 0.05M Phosphate Buffer at pH 6.0, less than or equal to 20% of 8-(2,6-difluorophenyl)-4-(4-fluoro-2-methylphenyl)-2-{[2-hydroxy-1-(hydroxymethyl)ethyl]-amino}pyrido[2,3-d]-pyrimidin-7(8H)-one tosylate released after 1 hour, from 29 to 69% of 8-(2,6-difluorophenyl)-4-(4-fluoro-2-methylphenyl)-2-{[2-hydroxy-1-(hydroxymethyl)-ethyl]-amino}pyrido[2,3-d]pyrimidin-7(8H)-one tosylate released after 7 hours and greater than 80% of 8-(2,6-difluorophenyl)-4-(4-fluoro-2-methylphenyl)-2-{[2-hydroxy-1-(hydroxymethyl)ethyl]-amino}pyrido[2,3-d]pyrimidin-7(8H)-one tosylate released after 18 hours.

In comparison, the conventional, immediate release 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one tablet of Example 1 dissolves 80% within 45 minutes. The dissolution profile was measured in a standard dissolution assay, for instance by the USP Basket method (USPI, chapter <711>), at 37.0+−0.5 degree ° C., using 0.01M hydrochloric acid or other suitable media (500 ml) and a rotation speed of 75 rpm.

### Polymorphic Forms

Another aspect of the invention are the novel polymorphic forms of 4-methylbenzenesulphonate (tosylate) salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one.

In another embodiment of the invention is a process for the preparation of the polymorphic Forms 1 to 4. In particular an embodiment of the invention is the preparation of Form 4 which comprises:
a) obtaining pure or substantially pure 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate; and
b) crystallizing 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate from an appropriate solvent under conditions which lead to the formation of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate Form 4.

For purposes herein, Form 1 and Form I, Form 2 and Form II, Form 3 and Form III, and Form 4 and, Form IV are used interchangeably. Also, form I and Form 1, form 2 and Form 2, form 3 and Form 3, form 4 and Form 4 are also used interchangeably

The invention further provides for mixtures of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate which comprise form 1, form 2, form 3, and form 4. In one embodiment the mixture may include both form 1 and form 4. The composition may comprise from 1, 2, 3, 4, 5, 10, 15, 20, 25, 30, 35, 40, 45, 50, 55, 60, 70, 75, 80, 85, 90, 95, 97 or greater than about 99 percent of either Form 1 or Form 4. In another embodiment the mixture may comprise 1, 2, 3, 4, 5, 10, 15, 20, 25, 30, 35, 40, 45, 50, 55, 60, 70, 75, 80, 85, 90, 95, 97 or greater than about 99 percent of either Form 1 or Form 3. In another embodiment the mixture may comprise 1, 2, 3, 4, 5, 10, 15, 20, 25, 30, 35, 40, 45, 50, 55, 60, 70, 75, 80, 85, 90, 95, 97 or greater than about 99 percent of either Form 3 or Form 4.

In one embodiment of the invention a composition may comprise from 1, 2, 3, 4, 5, 10, 15, 20, 25, 30, 35, 40, 45, 50, 55, 60, 70, 75, 80, 85, 90, 95, 97 or greater than about 99 percent of an individual polymorphic form, be it Form 1, Form 2, Form 3, or Form 4.

In another embodiment of the invention a composition may comprise one or more polymorphic forms as described herein and an amorphous form of the tosylate compound.

As is known, the crystalline state of a compound can be described by several crystallographic parameters: unit cell dimensions, space groups, and atomic position of the atoms in the compound relative to the origin of its unit cell. These parameters are experimentally determined by crystal x-ray analysis. It is possible for a compound to form more than one type of crystal. These different crystalline forms are called polymorphs.

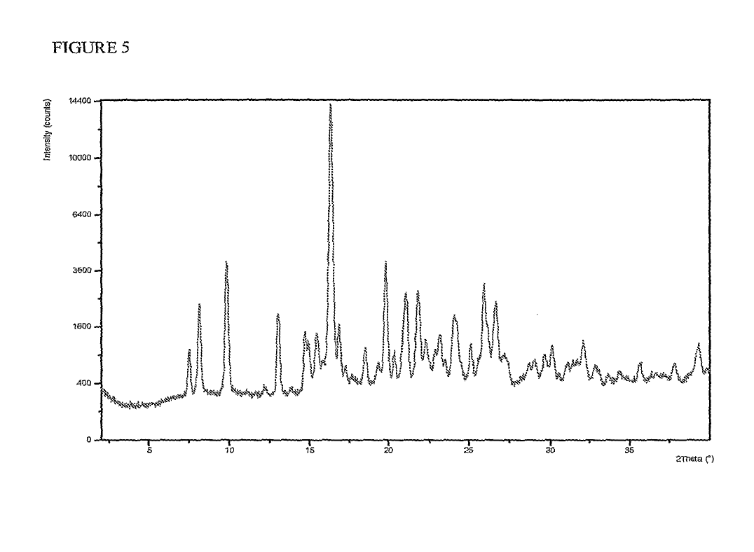

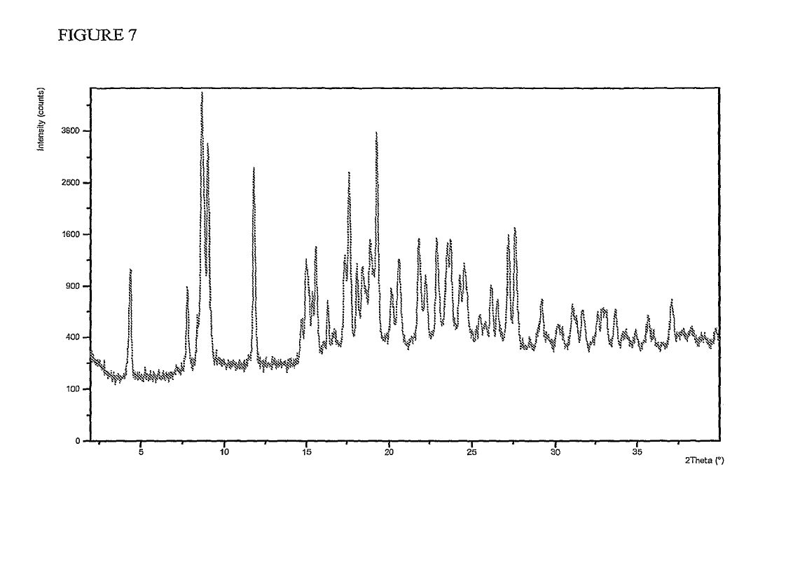

There have boon found to be 4 characterized, and reproducible polymorphic solid state forms of the 4-methylbenzenesulphonate (tosylate) salt of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one. These forms may be differentiated by X-Ray powder diffraction (XRPD) of the solid state forms, as shown herein in FIGS. 5 to 8 for Forms 1 to 4 respectively. FT-IR and Differential Scanning Calorimetry (DSC) data may also be used to assist in differentiation of the solid state forms as are shown and described herein.

Characteristic powder X-ray diffraction pattern peak positions are reported for polymorphs in terms of the angular positions (two theta) with an allowable variability, generally of about 0.1+/−°2-theta. The entire pattern, or most of the pattern peaks may also shift by about 0.1+/−° due to difference in calibration, setting, and other variations from instrument to instrument and from operator to operator.

The XRPD data described herein was acquired on a PANalytical X'Pert Pro powder diffractometer, model PW3040/60, serial number DY1850 using an X'Celerator detector. The acquisition conditions were: radiation: Cu Kα, generator tension: 40 kV, generator current: 45 mA, start angle: 2.0° 2θ, end angle: 40.0° 2θ, step size: 0.0167° 2θ, time per step: 31.75 seconds. The sample was prepared by mounting a few milligrams of sample on a Si wafer (zero background) plates, resulting in a thin layer of powder. Characteristic XRPD angles and d-spacings are recorded in Table 1 below. There are only a small number of peaks in the XRPD patterns which enable distinction between Forms 1, 2, 3 and 4.

Characteristic peak positions and calculated d-spacings are summarized in Table 1, and were calculated from the raw data using Highscore software. Peaks with a shaded background distinguish that form from the others. Other peaks (underscored and in bold) also distinguish the forms, however, there are shoulders or low intensity peaks of another form in close proximity that make these peaks less specific than those with a shaded background.

Polymorphic Form 1, may therefore be characterized by any one, any two, any three, any four, or any five or more of the 2-theta angle peaks. In particular, the peak at 8.2 2θ angle; or the peaks at 7.5 and 8.2 2θ angle. Use of the DSC thermograms and FT-IR may also assist in characterization of the polymorphs of the present invention.

Polymorphic Form 2, may therefore be characterized by any one, any two, any three, any four, or any five or more of the 2-theta angle peaks. In particular, the peaks at 3.7, and 7.2 2θ angle; or the peaks at 3.7, 7.2, and 11.7, 19.4 and 21.2.

Polymorphic Form 3, may therefore be characterized by any one, any two, any three, any four, or any five or more of the 2-theta angle peaks. In particular, the peak at 7.8 2θ angle; or the peaks at 4.4, 7.8, 8.7, 9.0 and 19.3 2θ angle.

Polymorphic Form 4, may therefore be characterized by any one, any two, any three, any four, or any five or more of the 2-theta angle peaks. In particular the peak at 8.0 2θ angle; or the peaks at 4.3, 8.0, 9.2, 16.7, 20.9, and 23.9 2θ angle.

Similar characterizations of any one, any two, any three, any four, or any five or more may also be attributed to the d-spacing/angstroms as shown in Table 1 below.

TABLE 1

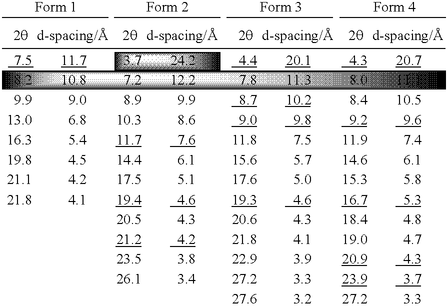

The FT-IR spectrum of the solid forms was recorded using a Nicolet Avatar 360 FT-IR spectrometer, serial number AEA0001623 fitted with a Diamond/ZnSe ATR Accessory at 4 cm⁻¹ resolution.

Form 1 bands were observed at:

3442, 3219, 3072, 2935, 1697, 1654, 1619, 1558, 1501, 1479, 1454, 1382, 1360, 1341, 1314, 1282, 1247, 1150, 1119, 1107, 1076, 1062, 1030, 1011, 1005, 983, 947, 913, 876, 838, 820, 798 and 709 cm⁻¹.

Suitably, Form 1 exhibits these characteristic bands of any one, any two, any three, any four, or any five or more bands.

Form 2 bands were observed at:

2950, 1703, 1654, 1622, 1554, 1499, 1480, 1451, 1360, 1319, 1289, 1238, 1183, 1155, 1117, 1076, 1052, 1029, 1007, 982, 943, 864, 848, 816, 797 and 710 cm⁻¹.

Suitably, Form 2 exhibits these characteristic bands of any one, any two, any three, any four, or any five or more bands.

Form 3 bands were observed at:

3369, 3076, 2963, 1705, 1653, 1624, 1574, 1559, 1501, 1477, 1455, 1360, 1314, 1286, 1278, 1231, 1183, 1156, 1141, 1119, 1101, 1069, 1030, 1006, 983, 964, 947, 885, 836, 818, 799 and 784 cm⁻¹.

Suitably, Form 3 exhibits these characteristic bands of any one, any two, any three, any four, or any five or more bands.

Form 4 bands were observed at:

3336, 3084, 1706, 1648, 1626, 1590, 1556, 1501, 1478, 1455, 1361, 1311, 1286, 1245, 1233, 1181, 1141, 1121, 1097, 1065, 1031, 1007, 981, 947, 865, 834, 818, 800, 781, 741 and 729 cm⁻¹.

Suitably, Form 4 exhibits these characteristic bands of any one, any two, any three, any four, or any five or more bands.

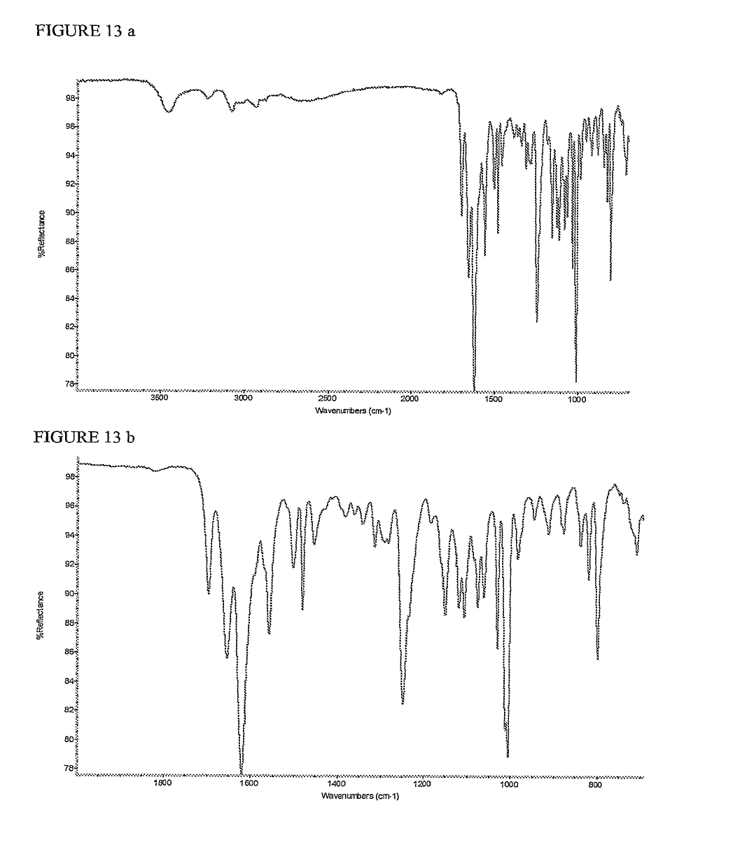

The IR data for Forms 1 to 4 is illustrated in FIGS. 13-16, respectively.

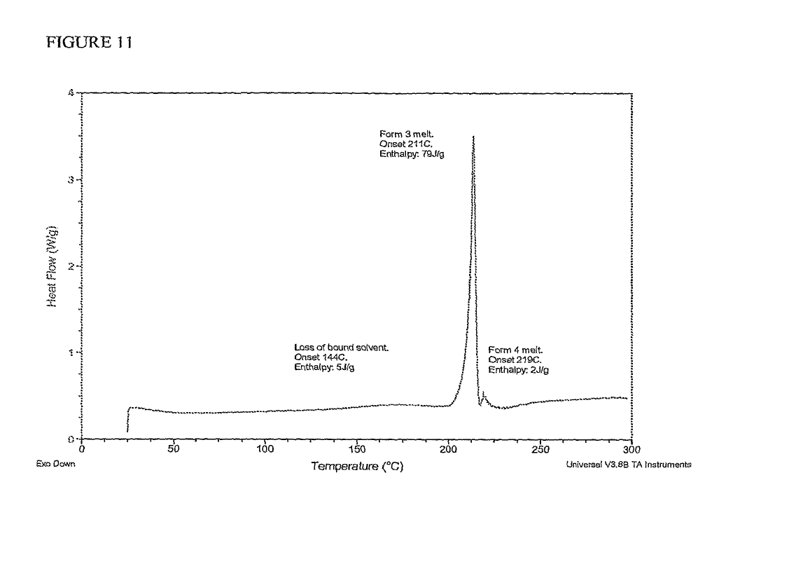

Form 4 is believed to be the most thermodynamically stable at room temperature with melt onset measured by DSC at approximately 218° C. Forms 1, 2 and 3 are less stable and show melt onsets of approximately 230° C., 206° C. and 211° C. respectively. In Forms 1 and 4, the melt event is followed by degradation. The melt enthalpy values, therefore, may not be accurate. The Form 2 melt may be followed by high temperature events. In the Form 3 trace, shown herein as FIG. 11, the Form 3 melt is followed by a small Form 4 melt.

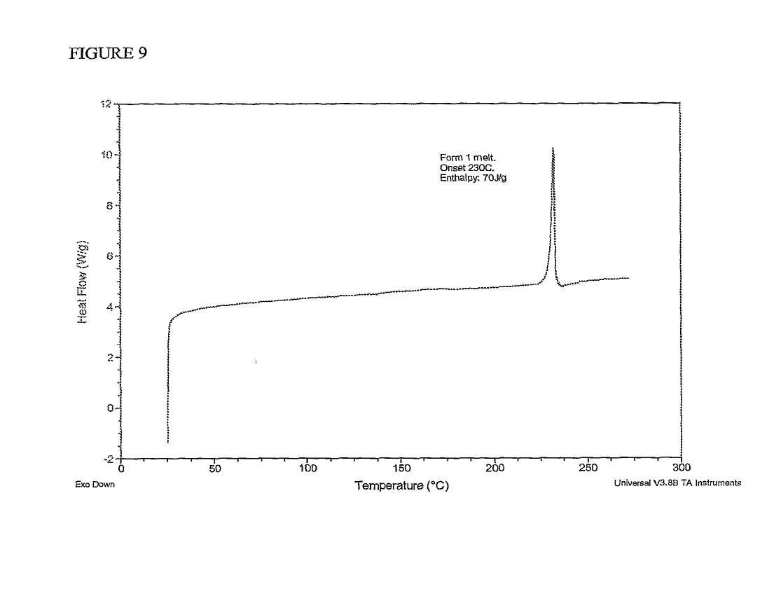

The DSC thermogram of the forms was obtained using a TA Instruments Q1000 calorimeter (instrument number: 970001.901, serial number: 1000-0126). The sample was weighed into an aluminium pan, a pan lid placed on top and lightly crimped without sealing the pan. The experiments were conducted using a heating rate of 10° C. min⁻¹. The data are illustrated herein as FIGS. 9-12 for Forms 1 to 4 respectively.

The solubility, ripening and melting point data indicate an enantiotopic system in which Form 4 is the more thermodynamically stable form at temperatures below about 135° C. and Form 1 is more thermodynamically more stable at temperatures above 135° C. (thus the higher melting point).

Thus, one embodiment of the invention is the polymorphic form, Form 1 substantially as shown in the X-ray diffraction pattern of FIG. 5, or differential scanning calorimetry thermogram of FIG. 9, or the infrared spectrum of FIG. 13 (a) and/or 13(b).

Another embodiment of the invention is the polymorph, Form 1 characterized by an x-ray diffraction pattern comprising peaks expressed in terms of 2 theta angles, wherein
i) said x-ray diffraction pattern comprises a peak at 8.2+/−0.1°; or
ii) said x-ray diffraction pattern comprises peaks at 7.5 and 8.2+/−0.1°; or
iii) said x-ray diffraction pattern comprises peaks at 8.2+/−0.1°, and 9.9+/−0.1°, or
iv) said x-ray diffraction pattern comprises peaks at 8.2+/−0.1°, and 13.0+/0.1°; or
v) said x-ray diffraction pattern comprises peaks at 8.2+/−0.1°, and 16.3+/−0.1°; or
vi) said x-ray diffraction pattern comprises peaks at 8.2+/−0.1°, and 19.8+/−0.1°; or
vii) said x-ray diffraction pattern comprises peaks at 8.2+/−0.1°, and 21.1+/−0.1°; or
viii) said x-ray diffraction pattern comprises peaks at 8.2+/−0.1°, and 21.8+/−0.1°; or
ix) said x-ray diffraction pattern comprises peaks at 7.5, 8.2, and 9.9+/−0.1°; or
x) said x-ray diffraction pattern comprises peaks at 7.5, 8.2, and 13.0+/−0.1°; or
xi) said x-ray diffraction pattern comprises peaks at 7.5, 8.2, and 16.3+/−0.1°; or
xii) said x-ray diffraction pattern comprises peaks at 7.5, 8.2, and 19.8+/−0.1°; or
xiii) said x-ray diffraction pattern comprises peaks at 7.5, 8.2, and 21.1+/−0.1°; or
xiv) said x-ray diffraction pattern comprises peaks at 7.5, 8.2, and 21.8+/−0.1°; or
xv) said x-ray diffraction pattern comprises peaks at 7.5, 8.2, 9.9, and 13.0+/−0.1°; or
xvi) said x-ray diffraction pattern comprises peaks at 7.5, 8.2, 9.9, 13.0, and 16.3+/−0.1°; or
xvii) said x-ray diffraction pattern comprises peaks at 7.5, 8.2, 9.9, 13.0, and 19.8+/−0.1°; or
xviii) said x-ray diffraction pattern comprises peaks at 7.5, 8.2, 9.9, 13.0, and 21.1+/−0.1°; or
xix) said x-ray diffraction pattern comprises peaks at 7.5, 8.2, 9.9, 13.0, and 21.8+/−0.1°; or
xx) said x-ray diffraction pattern comprises peaks at 7.5, 8.2, 9.9, 13.0, 16.3, 19.8, 21.1 and 21.8+/−0.1°.

Another embodiment of the invention is the polymorph, Form 1 having a powder X-ray diffraction pattern comprising a characteristic peak, in terms of 2θ at about 7.5+/−0.1° and 8.2+/−0.1°.

Another embodiment of the invention is the polymorph, Form 1 having a powder X-ray diffraction pattern comprising a characteristic peak, in terms of 2θ at about 7.5+/−0.1° and 8.2+/−0.1° and at least 1 additional characteristic peaks in terms of 2θ, selected from 9.9+/−0.1°, 13.0+/−0.1°, 16.3+/−0.1°, 19.8+/−0.1°, 21.1+/−0.1° and 21.8+/−0.1°.

Another embodiment of the invention is the polymorph, Form 1 having a powder X-ray diffraction pattern comprising a characteristic peak, in terms of 2θ at about 7.5+/−0.1° and 8.2+/−0.1° and at least 3 additional characteristic peaks in terms of 2θ, selected from 9.9+/−0.1°, 13.0+/−0.1°, 16.3+/−0.1°, 19.8+/−0.1°, 21.1+/−0.1° and 21.8+/−0.1°.

Another embodiment is the polymorph Form 1, or Form 2, or Form 3, or Form 4 in substantially pure crystalline form.

Another embodiment is wherein at least 30% by weight of total 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate polymorphic form, Form 1 in said composition is present. Suitably, at least 50%, at least 60, at least 70, at least 80, at least 90, at least 95, and at least 97% by weight of polymorph Form 1 is present.

Another embodiment is a pharmaceutical composition comprising polymorphic Form 1 of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate, and a pharmaceutically acceptable excipient or carrier.

Another embodiment of the invention is polymorph, Form 1 wherein said polymorph is characterized by a melt onset as determined by DSC of about 230° C.

Another embodiment of the invention is polymorph, Form 1 wherein said polymorph is characterized by a melt onset as determined by DSC of about 230° C., in combination with the infrared spectrum of FIGS. 13 (a) and/or 13(b).

Another embodiment of the invention is a process for the preparation of Form 1 from the tosylate salt in a solvent which is chloroform, a mixture of chloroform and an alcohol, such as methanol or ethanol, or methylene chloride and an alcohol, such as methanol or ethanol.

Another embodiment of the invention is a process for the preparation of substantially pure crystalline polymorph Form 1 comprising:

a) dissolving 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate in a suitable solvent, such as methylene chloride and a co-solvent and warming if necessary to give a solution;

b) cooling the solution of step (a), optionally in an ice bath or optionally seed the solution with crystalline tosylate form 1 to yield crystalline form 1.

Suitably, the cooling rate for large scale manufacture is about or up to 1° C./min.

In another embodiment, the tosylate salt is first suspended in chloroform or chloroform mixture and then cooled for formation of the crystalline form (optionally with seeding). A suitable co-solvent is methanol or ethanol. Alternatively chloroform may be used without a co-solvent as a slurry.

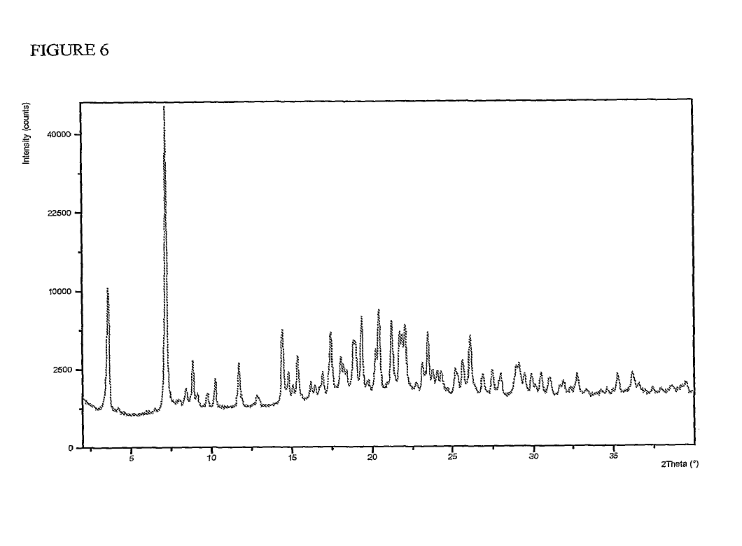

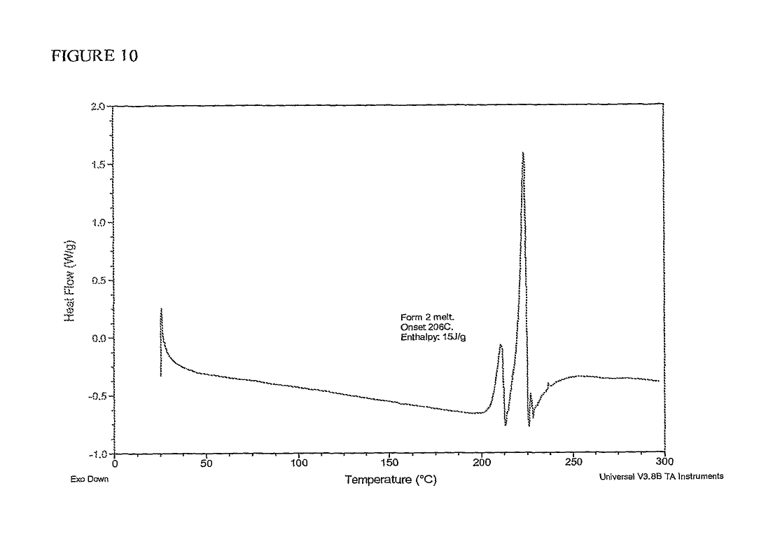

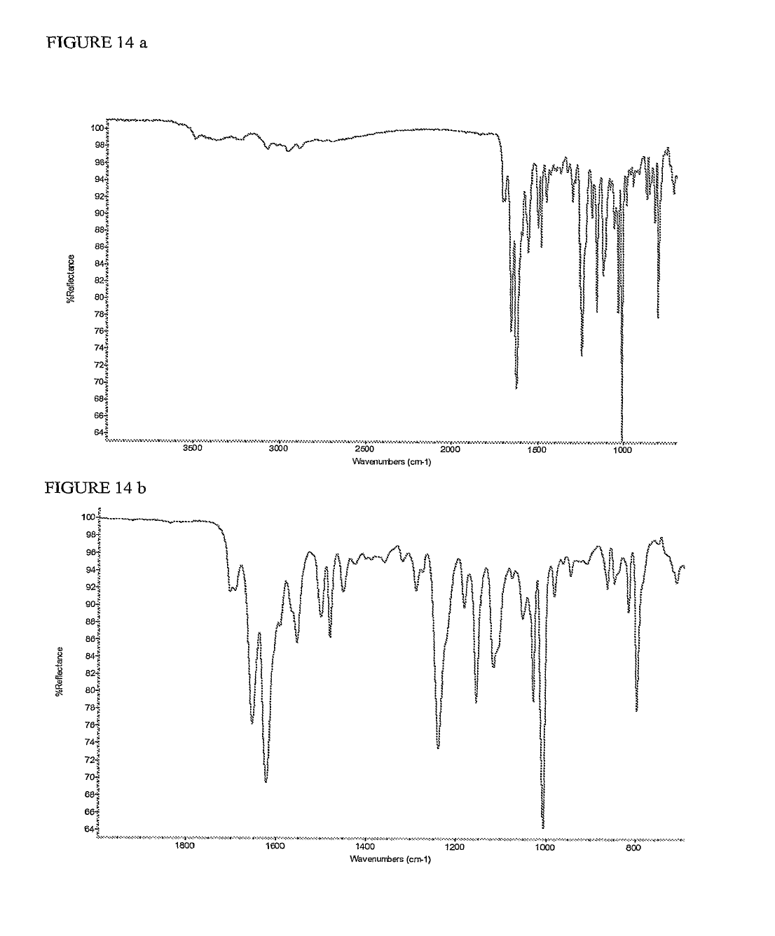

Another embodiment of the invention is the polymorph, Form 2, substantially as shown in the X-ray diffraction pattern of FIG. 6, or differential scanning calorimetry thermogram of FIG. 10, or the infrared spectrum of FIGS. 14(a) and/or 14(b).

Another embodiment of the invention is a composition comprising Form 2 wherein at least 30% by weight of total 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate in said composition is present as Form 2.

Another embodiment of the invention is a pharmaceutical composition comprising polymorphic Form 2 and a pharmaceutically acceptable excipient or carrier.

Another embodiment of the invention is polymorph, Form 2 wherein said polymorph is characterized by a melt onset as determined by DSC of about 206° C.

Another embodiment of the invention is a process for the preparation of Form 2 comprising crystallization of the tosylate salt by slow evaporation from a solvent mixture of acetonitrile and water.

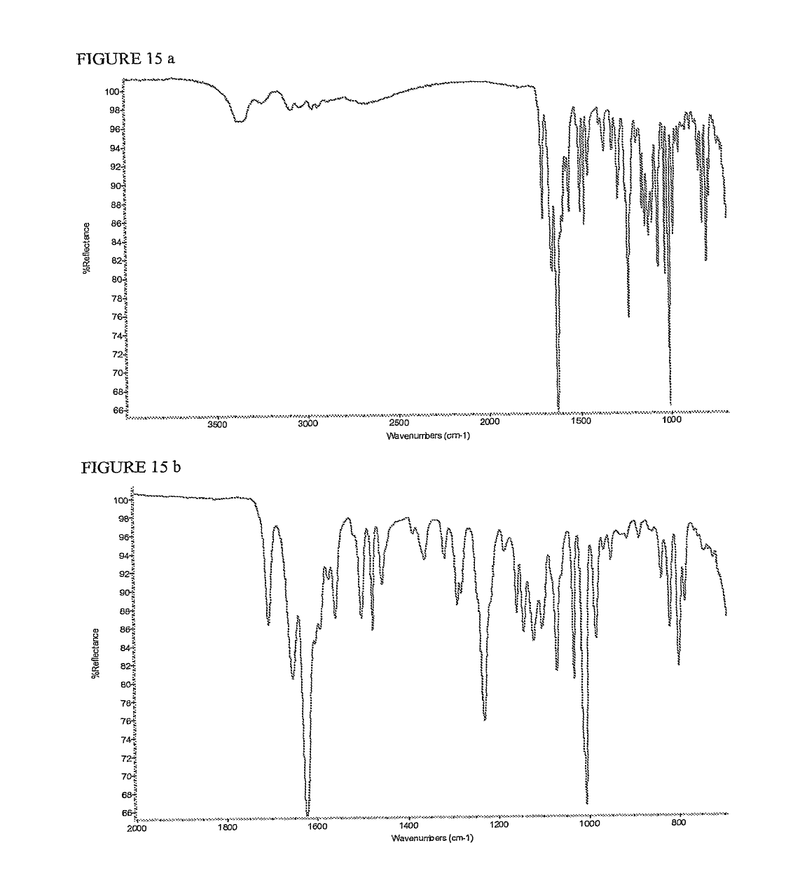

Another embodiment of the invention is the polymorph, Form 3, substantially as shown in the X-ray diffraction pattern of FIG. 5, or differential scanning calorimetry thermogram of FIG. 11, or the infrared spectrum of FIG. 15(a) and/or 15(b).

Another embodiment of the invention is a composition comprising Form 3 wherein at least 30% by weight of total 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate in said composition is present as Form 3.

Another embodiment of the invention is a pharmaceutical composition comprising polymorphic Form 3 and a pharmaceutically acceptable excipient or carrier.

Another embodiment of the invention is polymorph, Form 3 wherein said polymorph is characterized by a melt onset as determined by DSC of about 211° C.

Another embodiment of the invention is polymorph, Form 3 wherein said polymorph is characterized by a melt onset as determined by DSC of about 211° C. in combination with the infrared spectrum of FIGS. 15(a) and/or 15(b).

Another embodiment of the invention is a process for the preparation of Form 3 comprising crystallization of the tosylate salt by slow evaporation from methanol.

Alternatively, form 3 may be prepared by a slurry method of the tosylate salt in cyclohexane as a solvent at elevated temperatures, e.g. about 30° C., for an extended period of time, to yield Form 3.

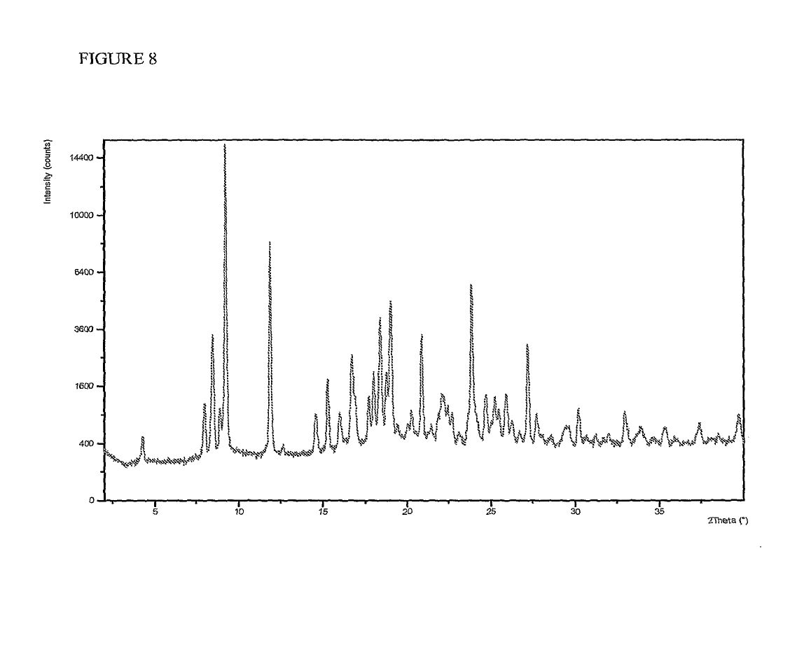

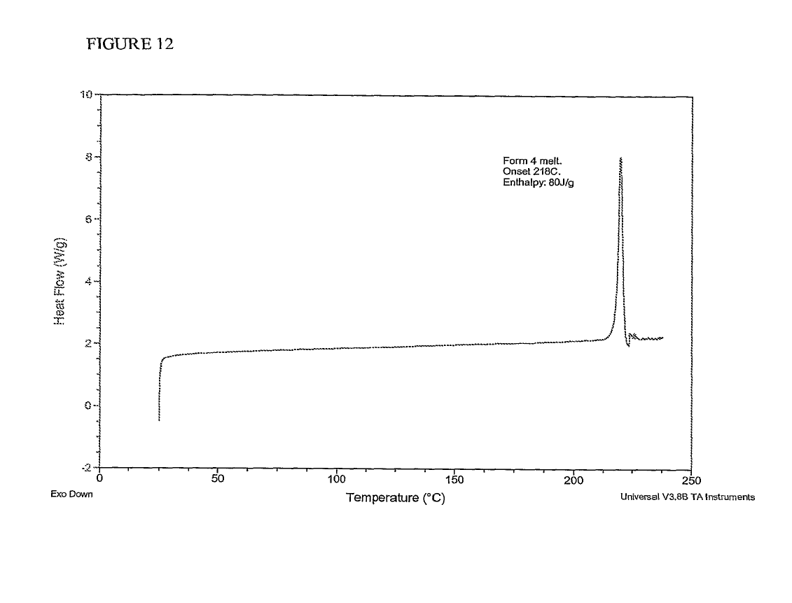

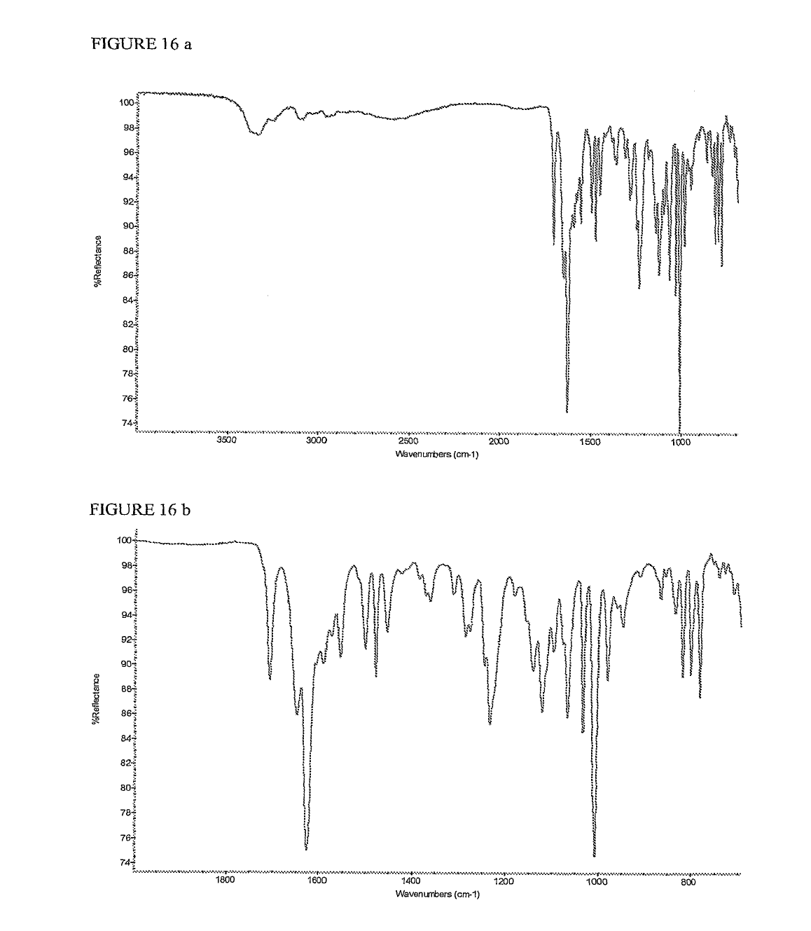

Another embodiment of the invention is the polymorphic form, Form 4, substantially as shown in the X-ray diffraction pattern of FIG. 8, or differential scanning calorimetry thermogram of FIG. 12, or the infrared spectrum of FIG. 16(a) and/or 16(b).

Another embodiment of the invention is polymorph form, Form 4 characterized by an x-ray diffraction pattern comprising peaks expressed in terms of 2 theta angles:
i) said x-ray diffraction pattern comprises a peak at 8.0+/−0.1°; or
ii) said x-ray diffraction pattern comprises peaks at 4.3+/−0.1° and 8.0+/−0.1°; or
iii) said x-ray diffraction pattern comprises peaks at 9.2+/−0.1° and 8.0+/−0.1°; or
iv) said x-ray diffraction pattern comprises peaks at 16.7+/−0.1° and 8.0+/−0.1°; or
v) said x-ray diffraction pattern comprises peaks at 20.9+/−0.1° and 8.0+/−0.1°; or
vi) said x-ray diffraction pattern comprises peaks at 23.9+/−0.1° and 8.0+/−0.1°; or
vii) said x-ray diffraction pattern comprises peaks at 4.3+/−0.1°, 8.0+/−0.1° and 9.2+/−0.1°; or
viii) said x-ray diffraction pattern comprises peaks at 4.3+/−0.1°, 8.0+/−0.1° and 16.7+/−0.1°; or
ix) said x-ray diffraction pattern comprises peaks at 4.3+/−0.1°, 8.0+/−0.1° and 20.9+/−0.1°; or
x) said x-ray diffraction pattern comprises peaks at 4.3+/−0.1°, 8.0+/−0.1° and 23.9+/−0.1°; or
xi) said x-ray diffraction pattern comprises peaks at 8.0+/−0.1°, 9.2+/−0.1°, and 16.7+/−0.1°; or
xii) said x-ray diffraction pattern comprises peaks at 8.0+/−0.1°, 9.2+/−0.1°, and 20.9+/−0.1°; or
xiii) said x-ray diffraction pattern comprises peaks at 8.0+/−0.1°, 9.2+/−0.1°, and 23.9+/−0.1°; or
xiv) said x-ray diffraction pattern comprises peaks at 8.0+/−0.1°, 16.7+/−0.1°, and 20.9+/−0.1°; or
xv) said x-ray diffraction pattern comprises peaks at 8.0+/−0.1°, 16.7+/−0.1°, and 23.9+A 0.1°; or
xvi) said x-ray diffraction pattern comprises peaks at 4.3+/−0.1°, 8.0+/−0.1°, 9.2+/−0.1°, and 16.7+/−0.1°; or
xviii) said x-ray diffraction pattern comprises peaks at 4.3+/−0.1°, 8.0+/−0.1°, 9.2+/−0.1°, and 20.9+/−0.1°; or
xix) said x-ray diffraction pattern comprises peaks at 4.3+/−0.1°, 8.0+/−0.1°, 9.2+/−0.1°, and 23.9+/−0.1°; or
xx) said x-ray diffraction pattern comprises peaks at 8.0+/−0.1°, 9.2+/−0.1°, 16.7+/−0.1°, and 20.9+/−0.1°; or
xxi) said x-ray diffraction pattern comprises peaks at 8.0+/−0.1°, 9.2+/−0.1°, 16.7+/−0.1°, and 23.9+/−0.1°; or
xxii) said x-ray diffraction pattern comprises peaks at 8.0+/−0.1°, 16.7+/−0.1°, 20.9+/−0.1° and 23.9+/−0.1°; or
xxiii) said x-ray diffraction pattern comprises peaks at 8.0+/−0.1°, 9.2+/−0.1°, 16.7+/−0.1°, and 23.9+/−0.1°; or
xxiv) said x-ray diffraction pattern comprises peaks at 4.3+−0.1°, 8.0+/−0.1°, 9.2+/−0.1°, and 20.9+/−0.1°; or
xxv) said x-ray diffraction pattern comprises peaks at 4.3+/−0.1°, 8.0+/−0.1°, 9.2+/−0.1°, and 23.9+/−0.1°; or
xxvi) said x-ray diffraction pattern comprises peaks at 4.3+/−0.1°, 8.0+/−0.1°, 9.2+/−0.1°, 20.9+/−0.1°, and 23.9+/−0.1°; or
xxvii) said x-ray diffraction pattern comprises peaks at 4.3+/−0.1°, 8.0+/−0.1°, 16.7+/−0.1°, 20.9+/−0.1°, and 23.9+/−0.1°; or
xxviii) said x-ray diffraction pattern comprises peaks at 4.3+/−0.1°, 8.0+/−0.1°, 9.2+/−0.1°, 16.7+/−0.1°, and 20.9+/−0.1°; or
xxix) said x-ray diffraction pattern comprises peaks at 8.0+/−0.1°, 9.2+/−0.1°, 16.7+/−0.1°, and 20.9+/−0.1°, and 23.9+/−0.1°; or
xxx) said x-ray diffraction pattern comprises peaks at 4.3+/−0.1°, 8.0+/−0.1°, 9.2+/−0.1°, 16.7+/−0.1°, 20.9+/−0.1° and 23.9+/−0.1°.

Another embodiment of the invention is polymorph form, Form 4 characterized by an x-ray diffraction pattern comprising peaks expressed in terms of 2 theta angles:
i) said x-ray diffraction pattern comprises a peak at 8.0+/−0.1°; or
ii) said x-ray diffraction pattern comprises peaks at 4.3+/−0.1° and 8.0+/−0.1°; or
iii) said x-ray diffraction pattern comprises peaks at 4.3+/−0.1°, 8.0+/−0.1° and 9.2+/−0.1°; or
iv) said x-ray diffraction pattern comprises peaks at 4.3+/−0.1°, 8.0+/−0.1°, 9.2+/−0.1°, and 16.7+/−0.1°; or
v) said x-ray diffraction pattern comprises peaks at 4.3+/−0.1°, 8.0+/−0.1°, 9.2+/−0.1°, 16.7+/−0.1°, and 20.9+/−0.1°; or
vi) said x-ray diffraction pattern comprises peaks at 4.3+/−0.1°, 8.0+/−0.1°, 9.2+/−0.1°, 16.7+/−0.1°, 20.9+/−0.1° and 23.9+/−0.1°

Another embodiment of the invention is polymorph, Form 4 having a powder X-ray diffraction pattern comprising a characteristic peak, in terms of 2θ at about 8.0+/−0.1° and at least 2 additional characteristic peaks in terms of 2θ, selected from 4.3+/−0.1°, 9.2+/−0.1°, 16.7+/−0.1°, 20.9+/−0.1° and 23.9+/−0.1°.

Another embodiment of the invention is polymorph, Form 4 having a powder X-ray diffraction pattern comprising a characteristic peak, in terms of 2θ at about 8.0+/−0.1° and at least 3 additional characteristic peaks in terms of 2θ, selected from 4.3+/−0.1°, 9.2+/−0.1°, 16.7+/−0.1°, 20.9+/−0.1° and 23.9+/−0.1°.

Another embodiment of the invention is polymorph, Form 4 wherein said polymorph is characterized by a melt onset as determined by DSC of about 218° C.

Another embodiment of the invention is polymorph, Form 4 wherein said polymorph is characterized by a melt onset as determined by DSC of about 218° C., in combination with the infrared spectrum of FIG. 16(a) and/or 16(b).

Another embodiment of the invention is wherein at least 30% by weight of total 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate in said composition is present in said composition as polymorph, Form 4. Suitably, at least 50%, at least 60, at least 70, at least 80, at least 90, at least 95, at least 97%, and at least 99% by weight of polymorph Form 4 is present.

Another embodiment of the invention is a composition comprising polymorphic Form 4 and a pharmaceutically acceptable excipient or carrier.

Another embodiment of the invention is a process for the preparation of substantially pure crystalline polymorph Form 4 comprising:

a) dissolving 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate in a suitable solvent, such as TBME: industrial methylated spirit (IMS), TMBE:IPA (9:1), or n-propanol, and warming if necessary to give a solution;

b) cooling the solution of step (a), optionally in an ice bath or optionally seed the solution with crystalline tosylate Form 4, to yield crystalline Form 4.

Suitably, the cooling rate for large scale manufacture is about or up to 1° C./min.

Another embodiment would be use other suitable solvents such as longer chain alcohols, e.g. butanol, isobutanol, or isopropanol, etc. or mixtures thereof, including TBME.

Preferably the solvent is TBME:IPA or n-propanol.

In another embodiment the crystallization method may first suspend the toyslate salt in a suitable solvent, such as tert-butylmethylether (TBME), toluene, butanol, or propanol, and then cooled for formation of the crystalline form (optionally with seeding). Another embodiment would be use other suitable solvents such as longer chain alcohols, e.g. isobutanol, or isopropanol, etc. or mixtures thereof, including TBME.

### Experimentals

Form 1 has been produced from a crystallization of methylene chloride and a co-solvent ethanol as demonstrated by Example Q, part (b) herein. Other solvents investigated which support the presence of Form I include crystallization from chloroform and chloroform/methanol (Example Q, part (c)), and those shown below.

Ostwald Ripening Experiments for Form 1: samples of the tosylate salt, were suspended and stirred in a designated solvent at a designated temperature for 3 to 5 days, then isolated and examined:
1) Using a mixture of form 1 and form 4, chloroform at 2° C., for 3 days produced DSC (231.9-233.8; dH=79.5 J/g) and XRPD consistent with support for Form
2) Using a mixture of form 1 and form 4, tetrahydrofuran at 2° C., for 3 days produced DSC 229.9-231.3; dH=85 J/g) and XRPD consistent with support for Form 1.
3) Using form 1, in water at 2° C. for 4 days, produced an XRPD shown to match form 1.
   Slow Evaporation Procedures that Gave Form 1 Material:

Samples of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate were dissolved in designated solvent at room temperature, filtered, an allowed to slowly evaporate at atmospheric pressure until solids appeared, then isolated and examined:
1) a stirred mixture of Form 1 tosylate salt was dissolved in a solvent, such as chloroform and the solvent was evaporated without stirring to produce a DSC and XRPD consistent with support for Form 1.

### Slow Evaporation Procedures for Form 2 Material:

Samples of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate form 1, was treated with acetonitrile/water 80/20 (vol/vol) until all solids dissolved. The clear solution was filtered to ensure no seed crystals remained. The clear solution was evaporated at room temperature under atmospheric pressure until solids appeared. The solids were isolated by filtration and analyzed. The solid was deemed to be form 2 by XRPD and DSC.

### Slow Evaporation Procedures for Form 3 Material:

Samples of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate form 1 were treated with MeOH until all solids were dissolved. The clear solution was filtered to ensure no seed crystals remained. The clear solution was evaporated at room temperature under atmospheric pressure until solids appeared. The solids were isolated by filtration and analyzed. The solid was deemed to be form 3 by XRPD and DSC.

Ostwald Ripening Experiments for Form 3 material: samples of the tosylate salt were stirred in a designated solvent at a designated temperature for 3 to 5 days, then isolated and examined:
1) a stirred mixture of Form 1 and Form 4 in cyclohexane at 30° C., for 5 days produced DSC and XRPD consistent with support for Form 3

### Form 4

There are many methods for the preparation of form 4. This has been deemed the thermodynamically most stable form at room temperature. It is enantiotropic with Form 1 with a cross-over temperature of about 135° C., a number which has been determined experimentally.

Crystallization from N-Propanol

As can be determined by a number of the working examples herein, and specifically that of Example R and S, 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one tosylate may be crystallized as form 4 from n-propanol; with seeding such as shown in Example D; and with seeding from TBME:TPA in Example C herein.

Ostwald Ripening Experiments for Form 4 material: samples of the tosylate salt were stirred in a designated solvent at a designated temperature for 3 to 5 days, then isolated and examined:
1) Stirred mixture of Form 1 and Form 4 in Tert-butylmethylether at 0° C. for 3 days support form 4 as determined by DSC and XRPD.
2) Stirred mixture of Form 1 and Form 4 in toluene at 0° C. for 3 days support form 4 as determined by DSC and XRPD.
3) Stirred mixture of Form 1 and Form 4 in toluene at 35° C. for 4 days support form 4 as determined by DSC and XRPD.
4) Stirred mixture of Form I and Form IV in 1-butanol at 30° C. for 5 days support form 4 as determined by DSC and XRPD.
5) Stirred mixture of Form I and Form IV in 1-butanol at 2° C. for 5 days support form 4 as determined by DSC and XRPD.
6) Stirred mixture of Form I and Form IV in 1-propanol at 0° C. for 4 days support form 4 as determined by DSC and XRPD.

### Amorphous Material

A non-crystalline solid form of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one tosylate has also been determined.

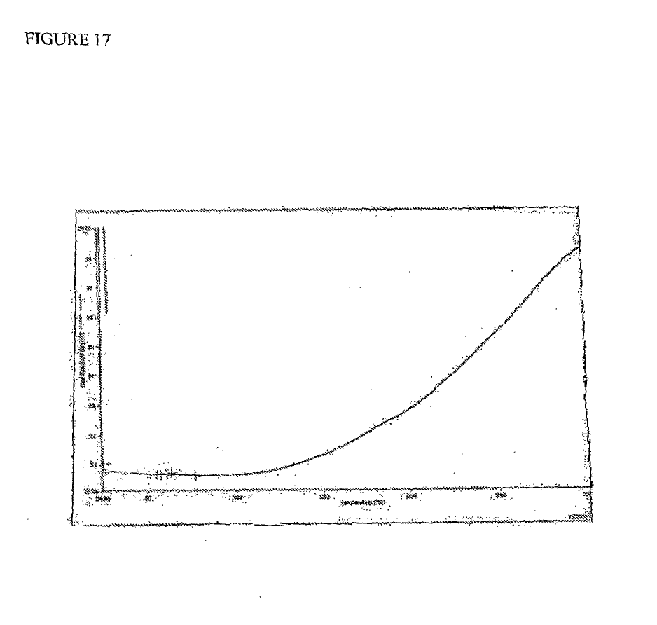

Ostwald Ripening Experiments for Amorphous material: samples of the tosylate salt were stirred in a designated solvent at a designated temperature for 3 to 5 days, then isolated and examined:
1) Using a mixture of Form 1 and Form 4 in water at 30° C. for 5 days, produces amorphous product as determined by XRPD and DSC (FIG. 17); the data suggests that some form 4 material remains, with the a predominate amorphous content.
2) Using a mixture of Form 1 and Form 4 in THF/water at a 1/10 ratio at 30° C. for 7 days, produced amorphous material a produces amorphous product as determined by XRPD and DSC; the data suggests that some form 4 material remains, with the a predominate amorphous content.

Thus another aspect of the invention is the amorphous form of -(2,6-difluorophenyl)-4-(4-fluoro-2-methylphenyl)-2-{[2-hydroxy-1-(hydroxymethyl)ethyl]amino}pyrido-[2,3-d]pyrimidin-7(8H)-one tosylate, and a pharmaceutical composition comprising the amphorous form and a pharmaceutically acceptable carrier or dituent.

### DEFINITIONS AND CONVENTIONS

The definitions and explanations below are for the terms as used throughout this entire document including both the specification and the claims.

### Definitions

All temperatures are in degrees Centigrade.

TLC refers to thin-layer chromatography.

HPLC refers to high pressure liquid chromatography.

Saline refers to an aqueous saturated sodium chloride solution.

Chromatography (column and flash chromatography) refers to purification/separation of compounds expressed as (support; eluent). It is understood that the appropriate fractions are pooled and concentrated to give the desired compound(s).

IR refers to infrared spectroscopy.

IMS refers to industrial methylated spirit

NMR refers to nuclear (proton) magnetic resonance spectroscopy, chemical shifts are reported in ppm (delta) downfield from tetramethylsilane.

MS refers to mass spectrometry expressed as m/c, m/z or mass/charge unit. [M+H].+ refers to the positive ion of a parent plus a hydrogen atom. El refers to electron impact.

CI refers to chemical ionization. FAB refers to fast atom bombardment.

Eq or eq refers to equivalents

Ether refers to diethylether.

DIPEA refers to N,N-diisopropylamine, which is also known as Hunig's base

DBN refers to 1,5-diazabicyclo[4.3.0]onon-5-ene]

DCM refers to dichloromethane

THF refers to Tetrahydrofuran

IMS refers to Industrial Methylated Spirits

CLLE refers to Centrifugal Liquid Liquid Extraction

NMP refers to N-Methyl 2-Pyrrolidinone

M refers to molar

THF refers to tetrahydrofuran

LiOH refers to lithium hydroxide

H or h refers to hours

MTBE or TBME are interchangeable and refer to Tertiary Butyl Methyl Ether

a/a refers to area/area

ca. refers to approximately.

USP refers to United States Pharmacopeia.

cps refers to centipoises.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When solvent pairs are used, the ratios of solvents used are volume/volume (v/v).

When the solubility of a solid in a solvent is used the ratio of the solid to the solvent is weight/volume (wt/v).

### SYNTHETIC EXAMPLES

The invention will now be described by reference to the following examples which are merely illustrative and are not to be construed as a limitation of the scope of the present invention. All temperatures are given in degrees centigrade, all solvents are highest available purity and all reactions run under anhydrous conditions in an Argon atmosphere where necessary.

### Example A

### 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one

Using the procedures exemplified in International Application Number: PCT/US01/50493, International Published Number WO 02/059083 A2 published on Aug. 1, 2002, Example 64, the free base 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one may be obtained.

As will be further exemplified, the free base may also be made in accordance with the working examples and schemes herein.

### Example B

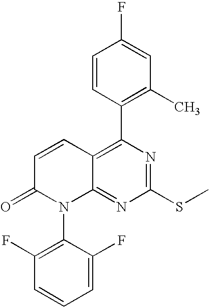

### Preparation of 8-(2,6-difluorophenyl)-4-(4-fluoro-2-methylphenyl)-2-(methylthio)pyrido[2,3-d]pyrimidin-7(8H)-one

4-[(2,6-difluorophenyl)amino]-6-(4-fluoro-2-methylphenyl)-2-(methylthio)-5-pyrimidinecarbaldehyde (18 g, 46 mmol), isopropylidene malonate (Meldrum's acid, 8.6 grams (hereinafter “g”), 60 millimoles (hereinafter “mmol”)) and anhydrous sodium acetate (3.4 g, 39 mmol) were stirred together in tetrahydrofuran (THF, 90 milliliters (hereinafter “mL”)) and the resultant mixture heated to 50-55° C. for 4 hours. The reaction temperature was reduced to 30° C., thioacetic acid (6.6 mL, 92 mmol) was added and the solution maintained at 30° C. for 16 hours. The resultant suspension was washed twice with 2 Molar (hereinafter “M”) aqueous sodium hydroxide (36 ml each wash) and then 10% w/v aqueous sodium chloride, industrial methylated spirit (IMS, 54 mL) and water (45 mL) were added, the solution was seeded, stirred for 2 hours and had more water (45 mL) added over 1 hour.

After 24 hours the slurry was filtered. The cake was washed with 3:3:5 THF:IMS:Water (36 mL) and twice with 20% aqueous IMS (2×36 mL) and dried in a vacuum oven to afford 8-(2,6-difluorophenyl)-4-(4-fluoro-2-methylphenyl)-2-(methylthio)pyrido[2,3-d]pyrimidin-7(8H)-one (14.5 g 761% th yield) 1H NMR (CDCl₃) δ: 7.49 (1H, m); 7.48 (1H, d, J=9.8); 7.28 (1H, d of d); 7.12 (2H, t, J=7.6); 7.00-7.10 (2H, m); 6.64 (1H, d, J=9.8); 2.26 (3H, s); 2.23 (3H, s).

4-[(2,6-difluorophenyl)amino]-6-(4-fluoro-2-methylphenyl)-2-(methylthio)-5-pyrimidinecarbaldehyde may be made by the route of Example 12 in WO 02/059083 or as described herein as Example F.

In a larger scale up, using similar conditions 8-(2,6-difluorophenyl)-4-(4-fluoro-2-methylphenyl)-2-(methylthio)pyrido[2,3-d]pyrimidin-7(8H)-one, was also obtained as follows:

To 4-[(2,6-difluorophenyl)amino]-6-(4-fluoro-2-methylphenyl)-2-(methylthio)pyrimidine-5-carbaldehyde (13.0 kilograms (hereinafter “kg”), 33.4 motes (hereinafter “mol”)), was added 2,2-dimethyl-1,3-dioxane-4,6-dione (Meldrum's acid, 6.26 kg, 43.4 mol) and anhydrous sodium acetate (2.2 kg, 39 mmol) and stirred together in tetrahydrofuran (65 Liters (hereinafter “L”)) with the resultant mixture heated to 50-55° C. for 4 hours. The reaction temperature was reduced to 30° C., thioacetic acid (5.1 kg, 92 mmol) was added and the solution maintained at 30° C. for 19 hours. The resultant suspension was washed twice with 2M aqueous sodium hydroxide (26 L each wash) and then 10% w/v aqueous sodium chloride (26 L). Industrial methylated spirit (39 L) and water (32.5 mL) were added, the solution was seeded, stirred for 2 hours and had more water (32.5 L) added.

After 16 hours the slurry was filtered. The cake was washed with 7:7:12 THF:IMS:Water (26 L) and twice with 20% aqueous IMS (2×26 L) and dried to give the title compound (10.4 kg 75% th yield). 1H NMR (CDCl₃) δ(ppm): 7.49 (1H, m, aromatic CH); 7.48 (1H, d, olefinic CH, J=9.8); 7.28 (1H, d of d, aromatic CH); 7.12 (2H, t, aromatic CH, J=7.6); 7.00-7.10 (2H, m, aromatic CH); 6.64 (1H, d, olefinic CH, J=9.8); 2.26 (3H, s, phenyl CH₃); 2.23 (3H, s, SMe)

In yet another variation the title compound was prepared: 4-[(2,6-difluorophenyl)amino]-6-(4-fluoro-2-methylphenyl)-2-(methylthio)-pyrimidine-5-carbaldehyde (Compound 1) (42 kg), sodium acetate (6.7 kg) and Meldrum's acid (20.2 kg) were heated in THF (126 L) at ca.62° C. for 3 hours. THF (210 L) was then added and the mixture was concentrated to 231 L via atmospheric distillation. The mixture was cooled to 32±2° C. and thioacetic acid (15.7 kg) was added slowly maintaining the contents temperature at 32±2° C. The mixture was then stirred at 32±2° C. for 10 hours.

The THF solution was cooled to 40±3° C. and washed with 2M sodium hydroxide solution (84 L) followed by 10% w/v potassium carbonate solution (2×84 L). The batch was cooled to 22±3° C., then isopropanol (126 L) and water (84 L) were added and the mixture was seeded with 8-(2,6-difluorophenyl)-4-(4-fluoro-2-methylphenyl)-2-(methylthio)pyrido[2,3-d]pyrimidin-7(8H)-one SB-691761 (0.42 kg). The solution was stirred at 22±3° C. for 1 hour. Further water (231 L) was added over 30 minutes at 22±3° C. The slurry was then cooled to 1±2° C. over 30 minutes and stirred at 112° C. for 30-60 minutes. The product was then collected by filtration. The filter cake was washed with isopropanol:water (4:1, 3×84 L) and the product was dried in vacuo at 60-65° C. to give the title compound (38.4 kg). 1H NMR (400 MHz, Chloroform-D) Tetramethylsilane as reference at 0.00 ppm. δ ppm 2.22 (s, 3H), 2.26 (s, 3H), 6.65 (d, J=9.78 Hz, 1H), 7.07 (td, J=8.62, 2.32 Hz, 2H), 7.14 (t, J=8.07 Hz, 2H), 7.28-7.30 (m, 1H), 7.47-7.53 (m, 2 H).

### Example C

### Preparation of 4-(4-fluoro-2-methylphenyl)-2-(methylthio)-7-oxo-8-(2,6-difluorophenyl)-7,8-dihydropyrido[2,3-d]pyrimidine-6-carboxylic acid

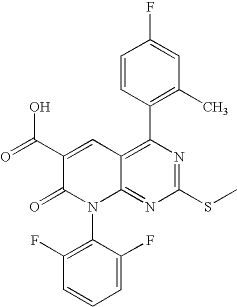

4-[(2,6-difluorophenyl)amino]-6-(4-fluoro-2-methylphenyl)-2-(methylthio)-pyrimidine-5-carbaldehyde (0.1 g, 0.26 mmol), 2,2-dimethyl-1,3-dioxane-4,6-dione (Meldrum's acid, 0.05 g, 0.35 mmol) and cesium acetate (0.029 g, 0.15 mmol) were stirred together in acetonitrile (1 ml) and the resultant mixture was heated to 50-55° C. for 1 hour. The mixture was stirred at ambient temperature overnight and water 0.3 mL was added. After stirring for 1 hour the mixture was filtered, the filter cake was washed with acetonitrile (2×0.5 mL) and dried to give the title compound (0.083 g, 69% th yield). 1H NMR (CDCl₃) δ(ppm): 2.28 (3H) s; 2.29 (3H) s; 7.09 (1H) t J=8.10 Hz; 7.12 (1H) d J=9.0 Hz; 7.20 (2H) t J=8.4 Hz; 7.28 (1H) dd J=8.8 and 3.2 Hz; 7.59 (1H) m; 8.71 (1H) s; 13.03 (1H) broad s;

### Example D

### Preparation of 8-(2,6-difluorophenyl)-4-(4-fluoro-2-methyl-phenyl)-2-{[2-hydroxy-1-(hydroxymethyl)ethyl]amino}pyrido[2,3-d]pyrimidin-7(8H)-one 4-methylbenzenesulfonate

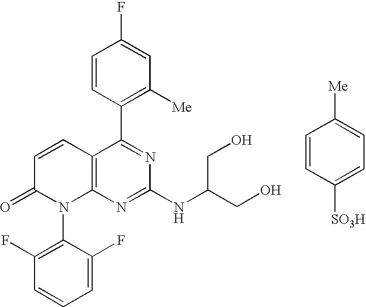

35% Hydrogen peroxide (7.6 L, 87 mol) was added at 18-21° C. to a stirred mixture of 8-(2,6-difluorophenyl)-4-(4-fluoro-2-methylphenyl)-2-(methylthio)-pyrido[2,3-d]pyrimidin-7(8R)-one (9.0 kg, 21.8 mol), tetrabutylammoniumhydrogen sulphate (370 g 1.1 mol), sodium tungstate dihydrate (140 g, 0.4 mol) in dichloromethane (46 L). Acetic acid (3.7 L) was then added and the mixture was stirred for 21 hours at 21° C. The phases were separated and the organic layer was washed with 1M sodium metabisulphite (18 L) and then water (2×18 L).

Dichloromethane (47 L) was added to the organic phase which was then concentrated to 45 L at atmospheric pressure. This solution was added to a solution of serinol (3.98 kg, 43.5 mol) in 1-methyl-2-pyrrolidone (NMP, 27 L) at 38° C. over 1 hour. After 1 hour the mixture was cooled to 19° C. and water (45 L) was added. The phases were separated and the organic phase was washed with water (4×36 L) before concentrating to 45 L by distillation at atmospheric pressure. Tert-butylmethyl ether (TBME, 35 L) was added to bring the volume to (80 L). TBME (230 L) was then added continuously with distillation at atmospheric pressure until the added volume had been removed. The organic solution (SOL) was diluted with isopropanol (IPA, 44 L) and TBME (19 L) and the temperature was adjusted to 48° C. To this warm solution was added 7 L of a solution of 4-methylbenzenesulfonic acid monohydrate (3.72 kg) in IPA (27 L) and TBME (27 L). The solution was seeded (20 g) with form 4, and the remaining 4-methyl-benzenesulfonic acid solution was added over 1 hour. The mixture was stirred for 1 hour at 48° C. before cooling to 20° C. at 1 deg° C./min. After stirring for 15 hours, the mixture was filtered, the filter cake was washed with TBME:IPA (9:1, 36 L) and TBME (2×36 L) and dried to give the title compound (10.7 kg, 80% th yield), (m.p. 215° C. by DSC); 400 MHz NMR in DMSO-d6. DMSO-d5 as reference at 2.52 ppm. δ(ppm): 2.22 (1.5H) s, 2.25 (1.5H) s, 2.31 (3H) s, 3.28-3.51 (4.5H) n, 4.03 (0.5H) m, 6.32 (0.5H) d J=9.4 Hz, 6.34 (0.5) d J=9.7 Hz, 7.15 (2H) d J=7.9 Hz, 7.17-7.26 (1.5H) m, 7.30 (1H) d J=9.9 Hz, 7.32-7.40 (3H) m, 7.43 (1H) dd J=8.5 and 6.1, 7.52 (2H) d J=8.0, 7.60-7.72 (1.5H) m.

### Example E

### Preparation of 8-(2,6-difluorophenyl-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate

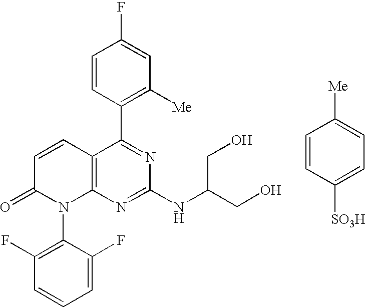

In an alternative crystallization to Example D above the following procedure has been devised: 30% Hydrogen peroxide (172 mL, 1.67 mol) was added at 18-21° C. to a stirred mixture of 8-(2,6-difluorophenyl)-4-(4-fluoro-2-methylphenyl)-2-(methylthio)pyrido-[2,3-d]pyrimidin-7(8H)-one (200 g, 0.48 mol), tetrabutylammoniumhydrogen sulphate (8.2 g, 24 mmol), sodium tungstate dihydrate (3.2 g, 9 mmol) in dichloromethane (1 L). After 20 h, acetic acid (86.6 mL) was added and the phases were separated and the organic layer was washed with 1M sodium metabisulphite and 5% aqueous sodium chloride in a continuous liquid-liquid extractor. 1-Methyl-2-pyrrolidone (NMP, 400 mL) was added, the solution was concentrated to 750 mL and added to a solution of serinol (88.4 g, 0.96 mol) in NMP (500 mL) at 42° C. over 1.5 h. After 2 h the solution was cooled to 20° C., washed with 5% aqueous sodium chloride in a continuous liquid-liquid extractor to remove NMP and distilled with the addition of 1-propanol (2.8 L) until all the dichloromethane was removed. 1-Propanol (300 mL) was added to bring the volume to 3 L and the solution was warmed to 75° C. To this warm solution was added a solution of 4-methylbenzenesulfonic acid monohydrate (82.6 g) in 1-propanol (500 mL). The solution was seeded (0.2 g) and the mixture was stirred for 2.5 h at 75° C. before cooling to 0° C.

The mixture was filtered, the filter cake was washed with 1-propanol (3×800 mL) and a portion dried to give the title compound (29.0 g). 400 MHz NMR in DMSO-d6. DMSO-d5 as reference at 2.52 ppm. δ(ppm): 2.22 (1.5H) s, 2.25 (1.5H) s, 2.31 (3H) s, 3.27-3.49 (4.5H) m, 4.01 (0.5H) m partly obscured by the water signal, 6.32 (0.5H) d J=9.7 Hz, 6.33 (0.5H) d J=9.5H, 7.14 (2H) d J=7.9 Hz, 7.21 (1H) tt J=8.5 and 2.0 Hz, 7.25-7.33 (1.5H) m, 7.33-7.46 (4H) m, 7.50 (2H) d J=8.0, 7.60-7.73 (1.5H) m

### Example F

### Preparation of 4-[(2,6-difluorophenyl amino]-6-(4-fluoro-2-methylphenyl)-2-(methylthio)-5-pyrimidinecarbaldehyde

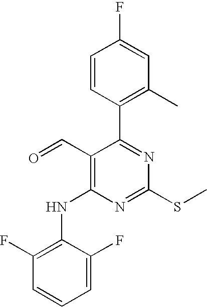

4,6-Dichloro-2-(methylthio)pyrimidine-5-carboxaldehyde (40 kg) was dissolved in anhydrous THF (200 L) at room temperature. Triethylamine (20 kg) was added followed by 2,6-difluoroaniline (26 kg) and the reaction mixture was heated at 60-65° C. for 12 h. The mixture was cooled to room temperature. To the mixture was added water (160 L) followed by triethylamine (25.6 kg), 4-fluoro-2-methylbenzeneboronic acid (33.2 kg), palladium acetate (0.5 kg, 2 mol %) and triphenylphosphine (1.88 kg, 4 mol %). The reaction mixture was heated to 65° C. and stirred vigorously at this temperature for 3 h. The mixture was cooled to 55-60° C. and THF (160 L) was added. The bottom aqueous phase was separated at 55-60° C. The organic phase was concentrated to 200 L, methanol (400 L) was added and the THF was removed (until <3 mol % THF by NMR) by atmospheric distillation, keeping the volume of the solution at ca.600 L by adding methanol (600-800 L). The solution was cooled to 55-60° C. and seeded with the title compound (0.16 kg). The slurry was stirred at 55° C. for at least 30 min and then cooled to 20° C. over 1 h. The slurry was stirred at 20° C. for at least 2 h and filtered. The cake was washed with methanol (160 L) followed by MeOH:water (4:1, 120 L) and dried in vacuo at 60-65° C. overnight to give the title compound (43.8 kg) 400 MHz NMR in CDCl₃. Tetramethylsilane as reference at 0.00 ppm. δ(ppm): 2.29 (3H) s, 2.33 (3H) s, 6.97-7.05 (4H) m, 7.24-7.32 (3H) m, 9.64 (1H) s, 10.38 (1H) s.

### Example G

### Preparation of 4-(2,4-difluorophenyl)-2-(methylthio)-8-(2,4,6-trifluorophenyl)pyrido[2,3-d]pyrimidin-7(8H)-one

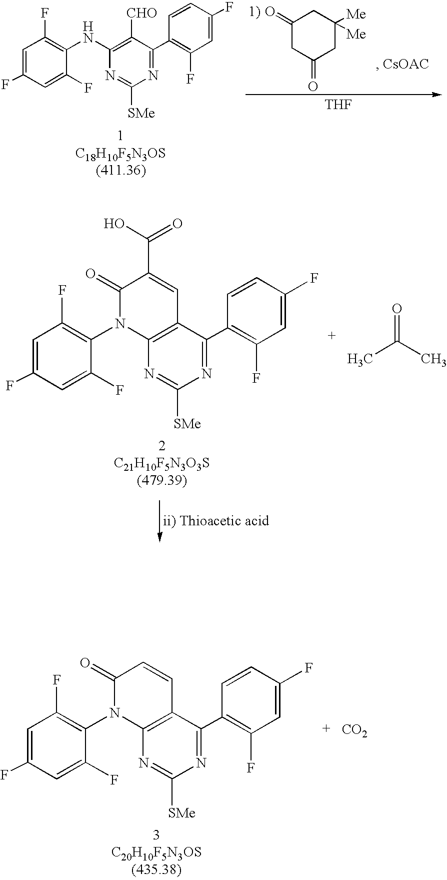

4-(2,4-difluorophenyl)-2-(methylthio)-6-[(2,4,6-trifluorophenyl)-amino]pyrimidine-5-carbaldehyde (1.35 Kg, 3.2 mol), 2,2-dimethyl-1,3-dioxane-4,6-dione (Meldrum's acid, 607 g, 4.2 mol) and anhydrous cesium acetate (257 g, 1.34 mol) were stirred together in tetrahydrofuran (6.75 L) and the resultant mixture was heated to 55±5° C. for 2.5 hours. The reaction mixture was cooled to 35° C. and thioacetic acid (229 mL, 3.2 mol) was added. After 2 hours the mixture was cooled to room temperature and stirred for a further 16 hours.

The mixture was washed with water (1.35 L), 1-propanol (2.03 L) was added to the organic phase followed by seed crystals and, after 15 minutes, water (675 mL) was added. After another 1 hour stir, more water (2.7 L) was added over 1 hour. After 1 hour stirring, more water (1.35 L) was added. After 16 hours stirring, the product slurry was filtered. The cake was washed twice with Industrial methylated spirit (2.7 L per wash) and dried in a vacuum oven at 55° C. to give the title compound (1.09 Kg, 76% th). 1H NMR (DMSO-d6) δ: 7.80 (1H, d, olefinic CH, J=9.8); 7.78 (1H, m, aromatic CH); 7.58 (2H, t, aromatic CH, J=9.1); 7.56 (1H, m, aromatic CH); 7.36 (1H, m, aromatic CH); 6.78 (1H, d, olefinic CH, J=9.8); 2.30 (3H, s, SMe).

Preparation of 4-(2,4-difluorophenyl)-2-(methylthio)-7-oxo-8-(2,4,6-trifluorophenyl)-7,8-dihydropyrido[2,3-d]pyrimidine may also be prepared as described in WO 03/088972, Example 1, part C.

### Example H

### Preparation of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate

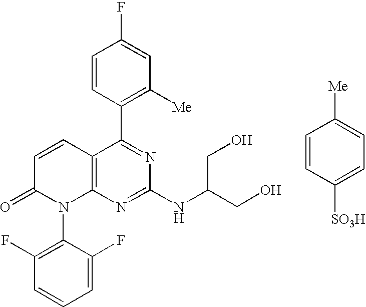

8-(2,6-difluorophenyl)-4-(4-fluoro-2-methylphenyl)-2-(methylthio)pyrido-[2,3-d]pyrimidin-7(8H)-one (18.0 kg) was dissolved in dichloromethane (90 L) and tetrabutylammonium hydrogensulphate (0.74 kg) and sodium tungstate dihydrate (0.3 kg) were added. 30% wt hydrogen peroxide (17 kg) was added over 2 h at 30° C. The reaction mixture was then stirred for 5 h at 30° C. and then cooled to 20±5 C. The bottom organic layer was separated and washed with 19% w/v aqueous sodium metabisulfite (90 L) and water (90 L). DCM (90 L) was added to the organics and the solution was concentrated at atmospheric pressure to 81 L. NMP (36 L) was added to give solution A. To a solution of serinol (9.9 kg) in NMP (45 L) at 42±2° C. was added solution A over 1 h. The mixture was stirred at 42±2° C. for 1 h and cooled to 20-25° C. Using CLLE, the above reaction solution was extracted into DCM with brine washing to remove the NMP. The DCM solution was concentrated to 72 L and n-propanol (234 L) was added. The solution was concentrated to 180 L, n-propanol (90 L) was added and the solution was heated to 80±5° C. A solution of 4-methylbenzene-sulfonic acid monohydrate (9.9 kg) in n-propanol (45 L) was added at 80±5° C. The solution was seeded with 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one 4-methylbenzenesulfonate (180 g) and stirred at 75±3° C. for 1.5 h before cooling to 1±3° C. The mixture was stirred at 1±3° C. for 1.5 h and then filtered. The filter cake was washed with n-propanol (3×72 L) and dried in vacuo at 70° C. to give the title compound (21.06 kg). 1H NMR (500 MHz, DMSO-d₆) Tetramethylsilane as reference at 0.00 ppm. δ ppm 2.22 and 2.25 (2×s, 3H), 2.30 (s, 3H), 3.34-3.43 (m, 2.5H), 3.48 (d, J=5.19 Hz, 2H), 4.04 (m, 0.5H), 6.33 (2×d, 1H), 7.14 (d, J=7.63 Hz, 2H), 7.17-7.25 (m, 1H), 7.29 (d, J=9.77 Hz, 1H), 7.33-7.40 (m, 3H), 7.41-7.45 (m, 1H), 7.52 (d, J=7.93 Hz, 2H), 7.61-7.69 (m, 2H).

### Example I

### Preparation of 3-[8-(2,6-difluorophenyl)-2-(methylthio)-7-oxo-7,8-dihydropyrido[2,3-d]pyrimidin-4-yl]-4-methylbenzoic acid

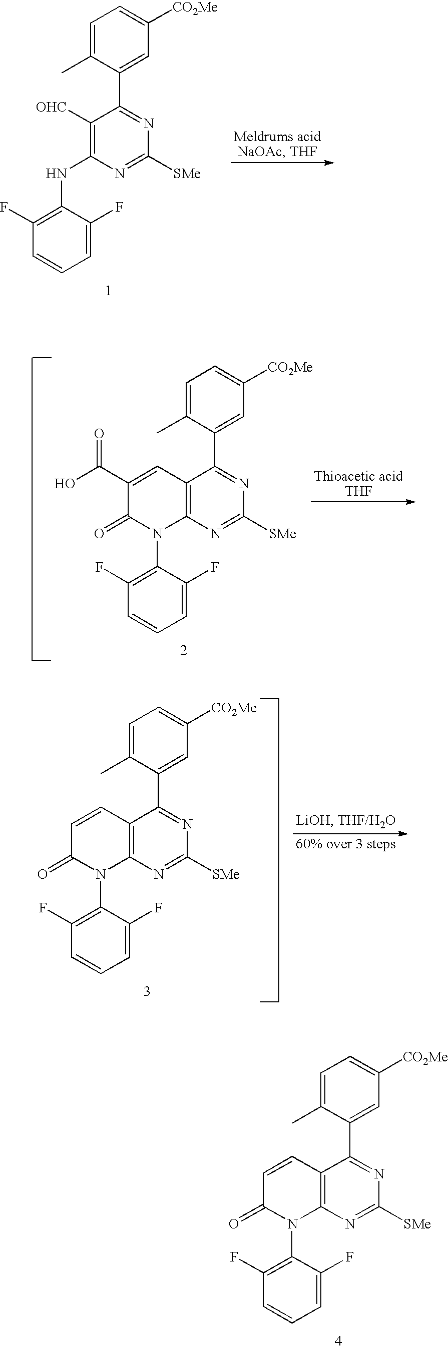

3-[6-(2,6-difluorophenyl)-amino]-5-formyl-2-methylthio-4-pyrimidinyl-4-methyl benzoate (30 kg), Meldrum's acid (13.1 kg), ground NaOAc (5.7 kg) and THF (135 L) were heated to reflux for about 6 to 12 hours. An additional 0.2 eq of NaOAc (1.1 kg) in water (4 L) was charged to the reaction mixture. The reaction was refluxed for about 12 h then additional Meldrum's acid (1.9 kg) in THF (ca. 6 L) was charged to the reaction mixture. The reaction mixture was refluxed for 13 hours. More Meldrum's acid (2.0 kg) in THF (6 L) was charged and the reaction was refluxed for 6 h.

Solvent was removed by vacuum distillation to adjust the volume to about 160 L. To the reaction mixture was charged thioacetic acid (5.83 kg) rinsed with THF (15 L). The reaction mixture was refluxed for 11 h and then cooled to room temperature. LiOH (11.7 kg) in water (140 L) was added and the reaction mixture was refluxed for 2-3 h. To the reaction mixture was charged water (130 L) and 50% NaOH (12 kg). Heptanes (150 L) were added to the reaction mixture for extraction. The organic layer was discarded. The aqueous layer was washed one more time with MTBE (150 L). The organic phase was again discarded. The pH of the aqueous was adjusted with 6M HCl to a target of pH≦2 (actual reading: pH=0.81). The aqueous was extracted twice with DCM (150 L). The combined organic phases were vacuum distilled down to 150 L. Heptanes (300 L) were added over a minimum of 4 h and then the mixture was then cooled to 0-5° C. and stirred at this temperature for 1 h. A further portion of heptanes (300 L) was added over 4 h. The product was collected by filtration washed, with heptanes (150 L) and dried on the filter to give the title compound (31.3 kg).

500 MHz NMR in DMSO-d6. TMS as reference at 0.00 ppm. δ(ppm): 2.25 (3H) s, 2.26 (3H) s, 6.72 (1H) d J=9.9 Hz, 7.42 (2H) dd J=8.6 and 8.6 Hz, 7.57 (2H) m, 7.57 (1H) d J=7.8 Hz, 7.71 (1H) m, 7.94 (1H) d J=1.4 Hz, 8.04 (1H) dd J=8.0 Hz and 1.6 Hz, 13.0 (1H) broad s.

### Example J

### Preparation of 4-(3-methyl-4-fluorophenyl)-2-(methylthio)-8-(2,6-trifluorophenyl)pyrido[2,3-d]pyrimidin-7(8H)-one

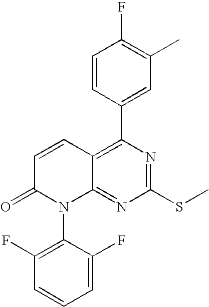

4-(3-Methyl-4-fluorophenyl)-2-(methylthio)-6-[(2,6-trifluorophenyl)amino]-pyrimidine-5-carbaldehyde (10.09 g, 25.9 mmol), 2,2-dimethyl-1,3-dioxane-4,6-dione (Meldrum's acid, 4.86 g, 33.7 mmol) and anhydrous sodium acetate (1.59 g, 19.4 mmol) were stirred together in tetrahydrofuran (30 mL) and the resultant mixture was heated to 60±5° C. for 6 hours. Tetrahydrofuran (50 mL) was added to the reaction and then solvent (30 mL) was distilled out of the reaction under atmospheric pressure. The reaction mixture was cooled to 34° C. and thioacetic acid (3.7 mL, 51.8 mmol) and tetrahydrofuran (50 mL) was added. After 24 hours at this temperature, solvent (40 mL) was distilled out under atmospheric pressure. The solution was cooled to room temperature before 2-propanol (30 mL) and water (20 mL) was added. The resulting solution was cooled slowly to 0° C. during which time precipitation occurred. After stirring for 90 minutes, the product slurry was filtered. The cake was washed three times with a mixture of 2-propanol and water (4:1, 3×20 mL) and dried in a vacuum oven at 50° C. to give the title compound (4.48 g, 84% th). 1H NMR (CDCl₃) δ(ppm): δ(ppm): 2.22 (3H) s, 2.39 (3H) s, 6.69 (1H) d, J=9.8 Hz, 7.12 (2H) t, J=8.1 Hz, 7.18 (1H) t, J=8.9 Hz, 7.45-7.51 (2H)m, 7.55 (1H) d, J=7.0 Hz, 7.90 (1H) d, J=10.1 Hz.

### Example K

### Preparation of 4-(2-methyl-5-fluorophenyl)-2-(methylthio)-8-(2,6-trifluorophenyl)-pyrido[2,3-d]pyrimidin-7(8H)-one

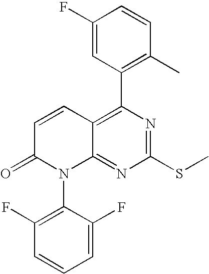

4-(2-Methyl-5-fluorophenyl)-2-(methylthio)-6-[(2,6-trifluorophenyl)-amino]pyrimidine-5-carbaldehyde (12 g, 30.8 mmol), 2,2-dimethyl-1,3-dioxane-4,6-dione (Meldrum's acid, 5.77 g, 40.1 mmol) and anhydrous sodium acetate (1.90 g, 23.1 mmol) were stirred together in tetrahydrofuran (36 mL) and the resultant mixture was heated to 64±5° C. for 3 hours. Tetrahydrofuran (60 mL) was added to the reaction and then solvent (36 mL) was distilled out of the reaction under atmospheric pressure. The reaction mixture was cooled to 33° C. and thioacetic acid (4.4 mL, 61.6 mmol) was added. After 23 hours at this temperature, the solution was cooled to room temperature and washed with 2M sodium hydroxide (24 mL) and then twice with 10% potassium carbonate solution (2×24 mL). 2-Propanol (36 mL) and water (24 mL) was added and then additional water (66 mL) was added over 1 hour. The resulting solution was cooled slowly to 0° C. After stirring for 1 hour, the product slurry was filtered. The cake was washed three times with a mixture of 2-propanol and water (4:1, 3×24 mL) and dried in a vacuum oven at 50° C. to give the title compound (8.50 g, 67% th). 1H NMR (CDCl₃) δ(ppm): 2.20 (3H) s, 2.23 (3H) s, 6.65 (1H) d, J=9.8 Hz, 7.04 (1H) dd, J=8.8, 2.7 Hz, 7.13 (2H) t, J=8.2 Hz, 7.32 (1H) dd, J=8.6, 5.6 Hz, 7.45-7.53 (2H) rm.

### Example L

### Preparation of 8-(2,6-difluorophenyl)-4-(2-methyl-5-(methoxycarbonyl)phenyl)-2-(methylthio)-7-oxo-7,8-dihydropyrido[2,3-d]pyrimidine-6-carboxylic acid

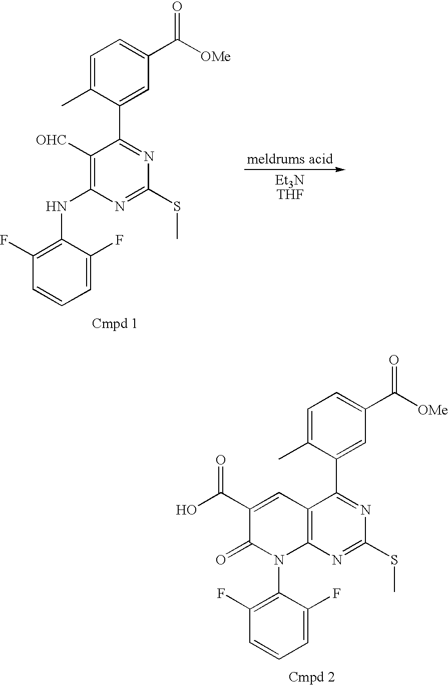

Triethylamine (0.52 ml, 3.7 mmol) was added at ambient to a stirred suspension of 3-[6-(2,6-difluorophenyl)-amino]-5-formyl-2-methylthio-4-pyrimidinyl-4-methyl benzoate (1.6 g, 3.7 mmol) and meldrums acid (0.7 g, 4.8 mmol, 1.3 eq) in THF (45 ml). The resultant yellow solution was stirred at ambient temperature for 2 hrs. Water (25 ml) was added and the mixture extracted into ethyl acetate (25 ml). The phases were separated and the organics washed with water (25 ml), dried over magnesium sulphate and concentrated to give the title compound (1.89 g). 400 MHz NMR in DMSO-d6. TMS as reference at 0.00 ppm. δ(ppm): 2.27 (3H) s, 2.29 (3H) s, 3.87 (3H) s, 7.45 (2H) t J=8.6 Hz, 7.63 (1H) d J=8.1 Hz, 7.73 (1H) m, 8.03 (1H) s, 8.03 (1H) d J=1.8 Hz, 8.08 (1H) dd J=8.0 and 1.8 Hz, 13.19 (1H) br s.

### Example M

### Preparation of 4-(2,4-difluorophenyl)-2-(methylthio)-7-oxo-8-(2,4,6-trifluorophenyl)-7,8-dihydropyrido[2,3-d]pyrimidine-6-carboxylic acid

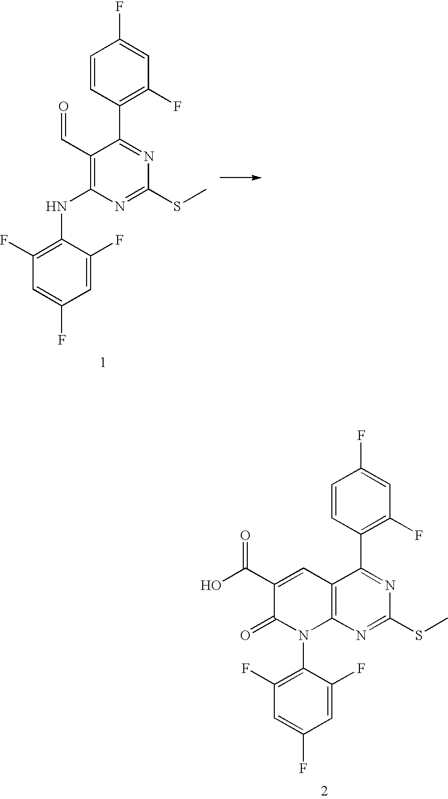

Compound 1, in the scheme above, 4-(2,4,6-trifluorophenyl)-6-(2,4-difluorophenyl)-2-methylthio)-5-pyrimidine carboxaldehyde (2.8 g, 1 eq) was stirred in DCM (28 ml) and pyridine (1.3 ml) with Meldrum's acid (0.97 g, 1 eq) overnight at room temperature. The resulting solution was heated at reflux until 6% starting material remained by HPLC and then DCM (15 ml) and 2M HCl (5 ml) were added. The DCM phase was washed with 2M HCL (5 ml) and the DCM evaporated off in vacuo. 1:1 McCN:H₂O (20 ml) was added to the residue which was stirred and heated at 65° to give a solid. The mixture was stirred and allowed to cool overnight and then filtered. The filter cake washed with 1:1 MeCN:H₂O (3×5 ml) and dried to give the title compound (20.4 g, 74% th yield). 1H NMR (400 MHz, Chloroform-D) Tetramethylsilane as reference at 0.00 ppm. δ ppm 2.35 (s, 3 H), 6.98 (t, J=8.07 Hz, 2H), 7.04-7.11 (m, 1H), 7.15 (t, J=8.07 Hz, 1H), 7.58-7.69 (m, 1H), 8.81 (d, J=4.16 Hz, 1H).

In an alternative embodiment, the reaction conditions were screened to use solvents and bases at higher temperatures than possible using DCM as shown above. Compound 1, 4-(2,4,6-trifluorophenyl)-6-(2,4-difluorophenyl)-2-methylthio)-5-pyrimidine carboxaldehyde (0.5 g) and Meldrum's acid (0.22 g, 1.25 eq) were heated at 60° for 3 h in either acetonitrile, toluene or ethyl acetate in the presence of DIPEA (0.1 ml, 0.5 eq) or potassium carbonate (0.08 g, 0.5 eq). All the reactions using DIPEA gave the title compound as did the use of potassium carbonate in ethyl acetate. With potassium carbonate in acetonitrile the reaction was very slow and in toluene no reaction was observed.

### Example N

### Preparation of 4-(2-methyl-4-fluorophenyl)-2-(methylthio)-8-(2,6-trifluorophenyl)pyrido[2,3-d]pyrimidin-7(8H)-one

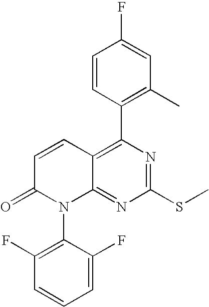

4-(2-Methyl-4-fluorophenyl)-2-(methylthio)-6-[(2,6-difluorophenyl)-amino]pyrimidine-5-carbaldehyde (5.30 g, 13.6 mmol), 2,2-dimethyl-1,3-dioxane-4,6-dione (Meldrum's acid, 2.55 g, 17.7 mmol) and anhydrous sodium acetate (0.84 g, 10.2 mmol) were stirred together in tetrahydrofuran (16 mL) and the resultant mixture was heated to 65±3° C. for 3 hours. The reaction mixture was cooled to 35° C. and potassium thioacetate (2.00 g, 17.7 mmol) was added. After 60 hours at this temperature, the solution was cooled to room temperature and washed with 5% potassium carbonate solution (21 mL) and then 10% potassium carbonate solution (10.5 mL). 2-Propanol (16 mL) and water (10.5 mL) was added and then additional water (29 mL) was added over 1 hour. The resulting solution was cooled slowly to 0° C. After stirring for 1 hour, the product slurry was filtered. The cake was washed three times with a mixture of 2-propanol and water (4:1, 3×10 mL) and dried in a vacuum oven at 50° C. to give the title compound (4.74 g, 84% th). 1H NMR consistent with previously prepared material.

### Example O

### Preparation of 4-(2,4-difluorophenyl)-2-(methylthio)-7-oxo-8-(2,4,6-trifluorophenyl)-7,8-dihydropyrido[2,3-d]-pyrimidine-6-carboxylic acid

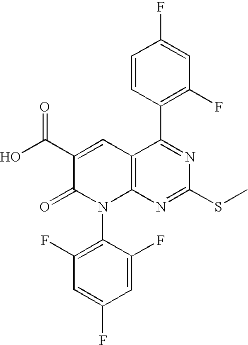

Additional experiments were run to screen alternative bases for use in the condensation reaction to obtain the title compound.

### Solid Bases

To 4-(2,4,6-trifluorophenyl)-6-(2,4-difluorophenyl)-2-methylthio)-5-pyrimidine carboxaldehyde (50 mg, 1 eq) was added Meldrum's acid (0.021 g, +/−3 mg, 1.2 eq) followed by one of the following bases and then acetonitrile (0.5 ml).
1) Cesium hydroxide monohydrate (0.035 g, 1.75 eq)
2) Lithium hydroxide (0.003 g, 1 eq)
3) Cesium carbonate (0.024 g, 0.6 eq)
4) Sodium hydroxide (0.005 g, 1 eq)
5) Lithium carbonate (0.007, 0.65 eq)
6) Calcium carbonate (0.0077 g, 0.63 eq)
7) Potassium bicarbonate (0.0114 g, 0.94 eq)
8) Sodium acetate (0.0049 g, 0.5 eq)
9) Magnesium carbonate (hydrated base) (0.0084 g, unknown eq)
10) Diphenylamine (0.0094 g, 0.5 eq)
11) Tetramethylpyrazine (0.0105 g, 0.5 eq)

The mixtures were stirred and heated at 55° for 4 hours.

### Liquid Bases

To 4-(2,4,6-trifluorophenyl)-6-(2,4-difluorophenyl)-2-methylthio)-5-pyrimidine carboxaldehyde (50 mg, 1 eq) was added Meldrum's acid (0.021 g, +/−3 mg, 1.2 eq) followed by a solution of one of the following bases and acetonitrile.
A) Triethylamine (0.03 ml) in acetonitrile (2 ml)
B) Hunig's base (0.04 ml) in acetonitrile (2 ml)
C) Pyridine (0.02 ml) in acetonitrile (2 ml)
D) 2,4,6-collidine (0.03 ml) in acetonitrile (2 ml)
E) Di-sec-butylamine (0.04 ml) in acetonitrile (2 ml)
F) 2,6-dimethylpiperidine in acetonitrile (2 ml)
G) Dihexylamine (0.06 ml) in acetonitrile (2 ml)
H) DBN (diazabicyclo[4.3.0]non-5-ene) (0.06 ml) in acetonitrile (2 ml)
I) 2,6-di-tert-butyl pyridine (0.03 ml) in acetonitrile (11 ml)
J) Isoquinoline (0.03 ml) in acetonitrile (2 ml)
K) N-methyl piperidine (0.03 ml) in acetonitrile (2 ml)
L) 2,6-Lutidine (0.03 ml) in acetonitrile (2 ml)
M) Pyrrolidine (0.0 ml) in acetonitrile (2 ml)

The mixtures were stirred and heated at 55° for 4 hour.

### Conclusion:

Inorganic bases: LiOH, NaOH, Li₂CO₃, CaCO₃, KHCO₃, MgCO₃ had no significant reaction; CsOH, and Cs₂CO₃ reacted with high impurities; sodium acetate had a clean reaction, almost complete.

Organic bases: tetramethylpyrazine, 2,6-di-tert-butylpyridine, diphenylamine had no significant reaction; Pyridine, di-sec-butylamine, isoquinoline, 2,6-lutidine had a partial reaction; DIPEA, triethylamine, DBN were complete but contained impurities; pyrrolidine, N-methylpiperidine, dihexylamine, dimethylpiperidine and 2,4,6-collidine had complete and reasonably clean reactions.

Additional larger scale reactions were run using potassium acetate, cesium acetate, and sodium acetate. 4-(2,4,6-trifluorophenyl)-6-(2,4-difluorophenyl)-2-methylthio)-5-pyrimidine carboxaldehyde (0.5 g), Meldrum's acid (0.23 g, 1.3 eq) and one of the 3 bases above (0.5 eq) were stirred and heated in acetonitrile at 60° All 3 acetates reacted cleanly with cesium being the quickest. Acetic acid addition to a sodium acetate reaction tube did not help product solubility.

### Example P

### Preparation of 4-(2,4-Difluorophenyl)-8-(2,4,6-trifluorophenyl)-2-(methylthio)pyrido[2,3-D]pyrimidine-7(8H)-one

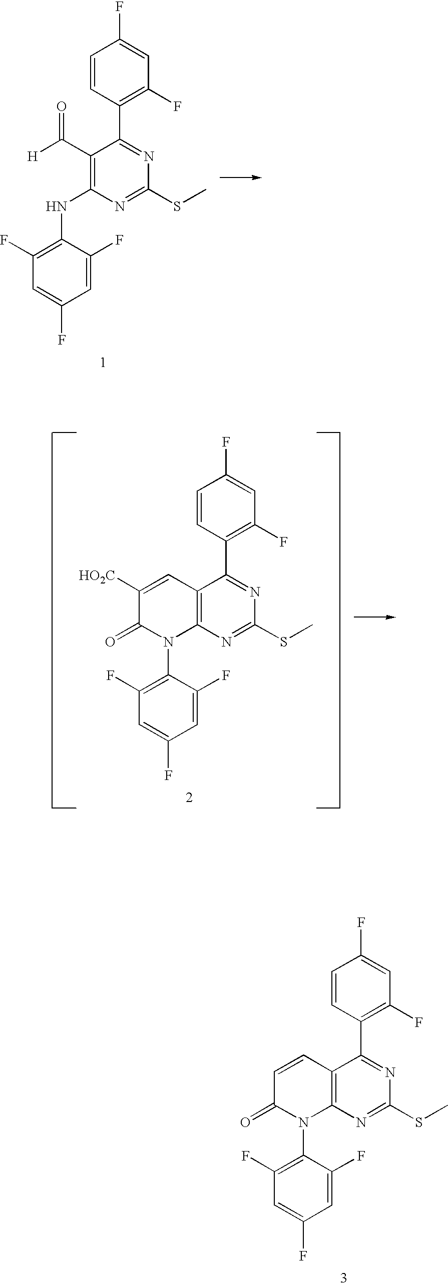

4-(2,4,6-trifluorophenyl)-6-(2,4-difluorophenyl)-2-methylthio)-5-pyrimidine carboxaldehyde (Compound 1 in the scheme above) (0.48 g, 1 wt., 1 eq), malonic acid (0.15 g, 1.2 eq, Aldrich) and toluene were stirred together. Piperidine (0.05 ml, 0.5 eq) was added to the mixture which was heated in an oil bath set at 95° C. After 1½ hours, pyridine was added to the reaction and heating continued overnight to give the title compound as the major product.

Alternatively in another experiment, the following were mixed together 4-(2,4,6-trifluorophenyl)-6-(2,4-difluorophenyl)-2-methylthio)-5-pyrimidine carboxaldehyde (100 mg). malonic acid (8 mg), piperidine (30 ml), acetic acid (5 ml), Ac₂O (5 ml) and DMF (360 ml). To the resulting mixture was added a further 260 ml of DMF. The solution was heat at 50° C. (bath) for 30 minutes. After 2½ hr, HPLC indicated 8:1 change.

The title compound is also produced in WO 03/088972, Example 1, part c whose disclosure is incorporated herein by reference.

In an alternative reaction to the above compound 1 (100 mg, 0.243 mmol, 1 eq), malonic acid (28 mg, 0.268 mmol, 1.1 eq), piperidine (30 ml), AcOH (5 ml), Ac₂O (5 ml), DMF (360 ml) were all mixed together. Further DMF (360 ml) was added to the solution and heated at 50° C. (bath) for 2.5 hours. HPLC indicated an 8:1 mixture of starting material and the title compound.

### Example Q

### Preparation of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate, form 1

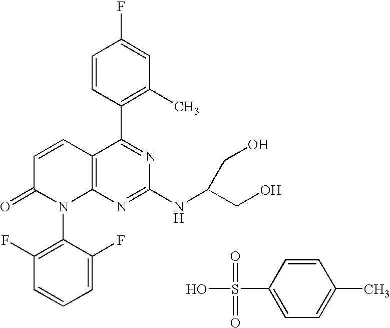

### (a) Acetonitrile Crystalline Polymorph

Three batches of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one (6.34 g; labeled “A”, 0.77 g; and labeled “B” 3.33 g-10.44 g total) were taken up in CH₃CN (30 mL) and added to a solution of p-toluenesulfonic acid monohydrate [4.75 g. (0.025 mol), Aldrich Chem.] with stirring. There was a very moderate exotherm; cooling gave crystals. A 1^{st} crop of 9.4 g (m.p. 217°-19° d.) was obtained; concentrating the filtrate to ⅓ volume afforded a 2^{nd} crop of 2.4 g (m.p. 217°-19° d.); a 3^{rd} crop of 0.8 g (m.p. 210°-12° d.) was obtained by cooling this filtrate overnight.

The 2^{nd} Crop (2.4 g) was stirred and sonicated with some acetone to give a uniform mixture. Solid was collected and dried. Wt. 1.5 g (m.p. 115°-16° d.).

The 1^{st} crop from above (9.4 g.), and the 1.5 g were combined and recrystallized from CH₃CN to give a white solid. Wt. 8.2 g. The material was pure (100%) by analytical HPLC with p-toluenesulfonic acid peak, LC/MS (100%) with p-toluenesulfonic acid peak; ¹H NMR (400 MHz, MeOD₄) δ 7.65-7.71 (m, 3H), 7.40-7.47 (m, 2H), 7.24-7.27 (m, 6H), 6.59 (s, 1H), 3.63 (s, 4H), 2.35-2.40 (m, 6H) LC MS (m/e)=457 (MH+) Rt=1.67 min. m.p. 217°-18° d. Anal. (C₃₀H₂₇F₃N₄O₆S) calcd: C, 57.32; H, 4.33; N, 8.91. found: C, 57.33; H, 4.18; N, 8.75

### (b) Chloroform Ethanol Ether Derived Crystalline Polymorph

8.1 g of the acetonitrile polymorph, described above, was taken up in CHCl₃ (300 mL) and, with stirring, warmed to a gentle reflux; sufficient co-solvent, EtOH (200 proof) was added to give a solution (about 6 μL). The clear solution was cooled with stirring and ethyl ether added to incipient turbidity. The mixture crystallized ‘en masse’. After cooling in an ice bath, the solid was collected and dried in vacuo to afford the product as a white solid. Wt. 7.9 g. The material was pure (100%) by analytical HPLC with p-toluenesulfonic acid peak, LC/MS (100%) with p-toluenesulfonic acid peak; ¹H NMR (400 MHz, DMSOd₆) δ 7.55-7.75 (m, 2H), 7.10-7.48 (m, 9H), 6.29-6.33 (m, 1H), 4.50-5.50 (bm, 2H), 3.99 (s, 1H), 3.30-3.35 (m, 5H), 2.29 (s, 3H), 2.22 (d, J=3.2 Hz, 3H) LC MS (m/e)=457 (MH+) Rt=1.67 min. m.p. 230°-31° d. Anal. (C₃₀H₂₇F₃N₄O₆S) calcd: C, 57.32; H, 4.33; N, 8.91. found: C, 56.99; H, 4.28; N, 8.82. mp=230-231° C. This sample was submitted for XRPD and designated as Form 1.

### Example R

### Preparation of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate, Form 4

4-methylbenzenesulfonic acid monohydrate (4.4 g) in 1-propanol (20 mL) was added at 75° C. to a solution of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one (8.83) in n-propanol (120 ml, prepared as described in Example H). The mixture was heated to 80° C. and the solution was stirred at this temperature until it crystallised (about 1.5 h). The suspension was then stirred and cooled to 0° C. and filtered. The filter cake was washed with n-propanol (4×32 ml) and dried to give the title compound (8.6 g) with an XRPD consistent with that of Form 4.

### Example S

### Preparation of 8-(2,6-difluorophenyl)-4-(4-fluoro-2-methylphenyl-2-{[2-hydroxy-1-(hydroxymethyl)ethyl]amino}pyrido[2,3-d]pyrimidin-7(8H)-one 4-methylbenzenesulfonate, Form 4

Charge a reactor with a solution of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one (15.0 g; 0.033 mole) in n-propanol (195 mL). Heat the contents of the reactor to about 80° C. Add a solution of p-toluene sulfonic acid monohydrate (6.6 g; 1.05 equivalent) in n-propanol (30 mL). Seed the solution at about 75° C. with Form 4 seeds. Cool to 0° C., and filter. The yield is 17.1 g of the form 4 tosylate salt, confirmed by DSC and XRPD.

### Example T

### Preparation of 8-(2,6-difluorophenyl-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate, Form 1

6 gm of the 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, hydrogen bromide salt was suspended in methanol (15 ml) and NaOH (1.5 gm in 10 ml) solution was added dropwise until pH 14 was achieved. The solution darkened, and the reaction was diluted with 50 ml of EtOAc, and 50 ml of water. The organics were washed with 50 ml of water and dried over sodium sulfate (Na2SO4) and filtered and concentrated to a foam (5.1 gm).

The para-toluene sulfonic acid (166 g) was dissolved in 35 ml of ACN. The free base (3.66 gm) was dissolved in a 105 ml of acetonitrile. The para-toluene sulfonic acid was added at room temperature to the free base solution. The reaction was allowed to stir at room temperature and was seeded with form 1. No crystallization occurred. The mixture was chilled in an acetone ice bath. After 1 hour the mixture was filtered and washed with acetonitrile and dried overnight to yield 3.5 gm of product.

3.5 gm of the product was suspended in 47 ml chloroform and 3 ml of methanol and heated to reflux to obtain complete dissolution and allowed to cool to room temperature. The precipitate was stirred for 60-minutes and filtered and washed with 10 ml of chloroform and air dried to give 2 gm of tosylate salt.

Product was analyzed by DSC and supports the presence of Form 1.

Chloroform has been found to make Form 1 at almost any temperature, e.g., from about 2° C. to about 40° C. in a slurry experimental basis.

### Example U

### Preparation of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate, form 1 and form 4 mixture

In a flask was added 6 gm of the 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate, hydrogen bromide salt suspended in methanol (15 ml). In another flask was added NaOH (1.5 gm dissolved in 10 ml of water) to yield a solution which was added dropwise to the HBr salt solution until pH 14 was achieved. 50 ml of EtOAc and 50 ml of water was added to the flask. The organics were dried over sodium sulfate (Na2SO4) and filtered and concentrated to a foam (5-7 gm) of the free base.

The free base (5.7 gm) was dissolved in 16 ml of acetonitrile. The para-toluene sulfonic acid (2.6 gm) was dissolved in 40 ml of acetonitrile. Both portions were combined and stirred. After 10 minutes a thick solution was observed. This was filtered and washed with acetonitrile to yield 2 gm of product. The filtrate was concentrated and combined with the 2 gm product and slurried in 50 ml of 96:4 chloroform:methanol v/v. The mixture was chilled but no precipitate was formed. 2 ml of ether was added and the product crystallized. The product was then filtered and washed with ether. The product was air-dried over weekend to yield 5 gm. Analysis by DSC and XRPD supports the presence of both form 1 and form 4.

### Example V

### Preparation of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate, Form 1

8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate (1 g, Form 4) was suspended in chloroform (15 ml) and temperature cycled at 0-40° C. for 3 days using the following cycle programme. Heat from 20° C. to 40° C. at 4° C./min, stir at 40° C. for 1 h, cool to 0° C. at 0.66° C./min, stir at 0° C. for 1 h, heat to 40° C. at 4° C./min. The mixture was filtered and the filter cake dried to give the title compound. XRPD and DSC analysis indicates Form 1.

### Example W

### Preparation of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate, Form 3

8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate (50 mg) was dosed into a reaction block. Methanol (750 μl) was manually dispensed and the slurry was set to shake at 500 rpm on a vortex mixer at 20° C. for 2 hours. The experiment was then filtered and the filtrate was evaporated to dryness under a flow of nitrogen. As the sample was dry after 2 hours under nitrogen, it was isolated for analysis. Raman, XRPD and DSC analysis indicate Form 3.

### Example X

### Preparation of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one 4-methylbenzenesulfonate Form 4

4-methylbenzenesulfonic acid monohydrate (4.4 g) in 1-propanol (20 mL) was added at 75° C. to a solution of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one (8.83) in n-propanol (120 ml, prepared as described in Example H). The mixture was heated to 80° C. and the solution was stirred at this temperature until it crystallised (about 1.5 h). The suspension was then stirred and cooled to 0° C. and filtered. The filter cake was washed with n-propanol (4×8 ml) and dried to give the title compound (8.6 g). XRPD and DSC analysis demonstrate form 4.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A sustained-release pharmaceutical composition comprising a water-soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, and a sustained release formulation.

2. The sustained release composition according to claim 1 wherein the salt is the tosylate salt.

3. The sustained release composition according to claim 1 wherein substantially all of the active agent is released from the formulation from about 2 to about 24 hours after administration to a patient.

4. The sustained release composition according to claim 1 which has an in vitro dissolution profile as determined by the basket apparatus of the USP (USP I, Chapter <711>) in which about 40 to about 60% of the active agent is dissolved after 3 hours.

5. The sustained release composition according to claim 1 which has an in vitro dissolution profile as shown in Examples 2 and 3 in FIG. 1; as shown in Examples 2, 3 and 4 in FIG. 2; or as shown in Examples 5 and 6 in FIG. 4.

6. The sustained release composition according to claim 1 which has an in vivo plasma concentration/time profile wherein the Area Under the Curve value is between 80% and 125% for each of Example 1 to 6 as depicted in FIG.

7.-17. (canceled)

18. An orally deliverable immediate release tablet comprising 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate in the form of a tablet.

19. The tablet according to claim 18 wherein the compound is present in an amount of 0.5 mg, 0.75 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg per tablet.

20. An orally deliverable tablet comprising a water-soluble salt of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one dispersed in a matrix comprising a hydrophilic polymer in the form of a tablet.

21. The tablet according to claim 20 wherein the water soluble salt is a tosylate salt.

22. The tablet according to claim 20 wherein the hydrophilic polymer is selected from at least one member of the group consisting of hydroxypropyl methylcellulose, hydroxyethyl cellulose, or hydroxypropyl cellulose.

23-24. (canceled)

25. The tablet of claims 23 wherein the hydroxypropyl methylcellulose is hydroxypropyl methylcellulose type 2208, or hydroxypropyl methylcellulose 2910, or mixtures thereof and is present in an amount of about 30% to about 45% by weight.

26. The tablet of claim 23 wherein the hydroxypropylmethylcellulose is hydroxypropyl methylcellulose type 2208 and is present in an amount of about 20% to about 25% by weight.

27. The compound 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate.

28. A pharmaceutical composition comprising the compound according to claim 27 and a pharmaceutically acceptable carrier or diluent.

29. The pharmaceutical composition according to claim 28 wherein the composition is in a unit dosage form for oral administration selected from a tablet, capsule, sachet, soft gel capsule, suspension, solution; or is a composition for topical administration selected from a liquid, gel, cream, suspension, or suppository; or is a composition for injection selected from an injectable gel, an intravenous, an intraocular, or intramuscular injectable.

30. A method of treatment, including prophylaxis, of a condition or disease state mediated by p38 kinase activity or mediated by cytokines produced by the activity of the p38 kinase comprising administering an effective amount of a compound or composition according to claim 27 to a mammal in need thereof.

31. The method according to claim 30 wherein the condition or disease state is rheumatoid arthritis, acute or chronic inflammatory disease states such as the inflammatory reaction induced by endotoxin or inflammatory bowel disease, atherosclerosis, neuropathic pain, chronic obstructive pulmonary disease (COPD), asthma, cystic fibrosis, and multiple myeloma.

32. A composition according comprising a compound according to claim 27 and a second therapeutic agent.

33. The composition according to claim 32 wherein the second therapeutic agent is a disease modifying antirheumatic drug selected from abatacept, entanercept, infliximab, adalimumab, methotrexate, hydroxychloroquine, sulfasalazi, leflunomide, Anakinra, rituximab, a corticosteroid, an NSAID, a COX-2 inhibitors, or a nonacetylated salicylates.

34. A compound of the formula: 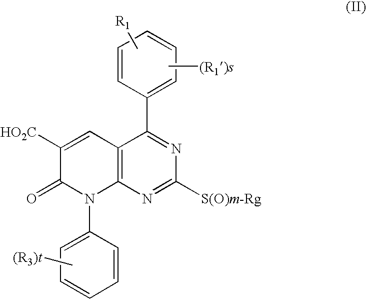
wherein
R₁ is independently selected from hydrogen, C(Z)N(R_{10′})(CR₁₀R₂₀)ᵥR_{b}, C(Z)O(CR₁₀R₂₀)ᵥR_{b}, N(R_{10′})C(Z)(CR₁₀R₂₀)ᵥR_{b}, N(R_{10′})C(Z)N(R_{10′})(CR₁₀R₂₀)ᵥR_{b}, or N(R_{10′})OC(Z)(CR₁₀R₂₀)ᵥR_{b};
R_{1′} is independently selected at each occurrence from hydrogen, halogen, C₁₋₄ alkyl, halo-substituted-C₁₋₄ alkyl, cyano, nitro, (CR₁₀R₂₀)_{v′}NR_{d}R_{d′}, (CR₁₀R₂₀)_{v′}C(O)R₁₂, SR₅, S(O)R₅, S(O)₂R₅, or (CR₁₀R₂₀)_{v′}OR₁₃;
R₃ is independently selected at each occurrence from hydrogen, halogen, C₁₋₄alkyl, or halosubstituted C₁₋₄alkyl;
R₄ and R₁₄ are each independently selected at each occurrence from hydrogen or C₁₋₄ alkyl, or R₄ and R₁₄ together with the nitrogen to which they are attached form a heterocyclic ring of 5 to 7 members, which ring optionally contains an additional heteroatom selected from NR₉;
R₅ is independently selected at each occurrence from hydrogen, C₁₋₄ alkyl, C₂₋₄ alkenyl, C₂₋₄ alkynyl or NR₄R₁₄, excluding the moieties SR₅ being SNR₄R₁₄, S(O)₂R₅ being SO₂H and S(O)R₅ being SOH;
R₉ and R_{9′} are independently selected at each occurrence from hydrogen, or C₁₋₄ alkyl;
R₁₂ is independently selected at each occurrence from hydrogen, C₁₋₄ alkyl, halo-substitutedC₁₋₄ alkyl, C₂₋₄ alkenyl, C₂₋₄ alkynyl, C₃₋₇cycloalkyl, C₃₋₇cycloalkylC₁₋₄ alkyl, C₅₋₇cycloalkenyl, C₅₋₇cycloalkenylC₁₋₄alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R₁₃ is independently selected at each occurrence from hydrogen, C₁₋₄ alkyl, halo-substituted C₁₋₄ alkyl, C₂₋₄ alkenyl, C₂₋₄ alkynyl, C₃₋₇ cycloalkyl, C₃₋₇cycloalkyl C₁₋₄ alkyl, C₅₋₇ cycloalkenyl, C₅₋₇cycloalkenyl C₁₋₄ alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R_{d} and R_{d′} are each independently selected at each occurrence from hydrogen, C₁₋₄ alkyl, C₃₋₆cycloalkyl, C₃₋₆cycloalkyl C₁₋₄alkyl moiety, and wherein each of these moieties, excluding hydrogen, may be optionally substituted; or R_{d} and R_{d′} together with the nitrogen which they are attached form an optionally substituted heterocyclic ring of 5 to 6 members, which ring optionally contains an additional heteroatom selected from oxygen, sulfur or NR_{9′};
R_{b} is hydrogen, C₁₋₁₀ alkyl, C₃₋₇ cycloalkyl, C₃₋₇ cycloalkyl C₁₋₁₀ alkyl, aryl, arylC₁₋₁₀alkyl, heteroaryl, heteroarylC₁₋₁₀ alkyl, heterocyclic, or heterocyclylC₁₋₁₀ alkyl moiety, which moieties, excluding hydrogen, may all be optionally substituted;
R_{g} is C₁₋₁₀ alkyl, or aryl;
m is 0 or an integer having a value of 1, or 2;
s is an integer having a value of 1, 2, 3 or 4; and
t is an integer having a value of 1, 2, 3 or 4.
v is 0 or an integer having a value of 1 or 2;
v′ is independently selected at each occurrence from 0 or an integer having a value of 1 or 2;
Z is independently selected from oxygen or sulfur;
R₁₀ and R₂₀ are independently selected at each occurrence from hydrogen or C₁₋₄ alkyl; and
R_{10′} is independently selected at each occurrence from hydrogen or C₁₋₄alkyl.

35. The compound according to claim 34 wherein R_{1′} is independently selected from halogen, or C₁₋₄alkyl.

36-47. (canceled)

48. The compound according to claim 34 which is
4-(4-fluoro-2-methylphenyl)-2-(methylthio)-7-oxo-8-(2,6-difluorophenyl)-7,8-dihydropyrido[2,3-d]pyrimidine-6-carboxylic acid;
4-(2,4-difluorophenyl)-2-(methylthio)-7-oxo-8-(2,4,6-trifluorophenyl)-7,8-dihydropyrido[2,3-d]pyrimidine-6-carboxylic acid; and
8-(2,6-difluorophenyl)-4-(2-methyl-5-(methoxycarbonyl)phenyl)-2-(methylthio)-7-oxo-7,8-dihydropyrido[2,3-d]pyrimidine-6-carboxylic acid.

49. A process for producing a compound of Formula (III) 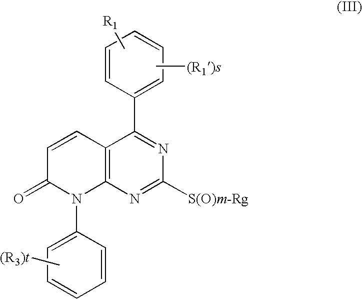
wherein
R₁ is independently selected from hydrogen, C(Z)N(R_{10′})(CR₁₀R₂₀)ᵥR_{b}, C(Z)O(CR₁₀R₂₀)ᵥR_{b}, N(R_{10′})C(Z)(CR₁₀R₂₀)ᵥR_{b}, N(R_{10′})C(Z)N(R_{10′})(CR₁₀R₂₀)ᵥR_{b}, or N(R_{10′})OC(Z)(CR₁₀R₂₀)ᵥR_{b};
R_{1′} is independently selected at each occurrence from hydrogen, halogen, C₁₋₄ alkyl, halo-substituted-C₁₋₄ alkyl, cyano, nitro, (CR₁₀R₂₀)_{v′}NR_{d}R_{d′}, (CR₁₀R₂₀)_{v′}C(O)R₁₂, SR₅, S(O)R₅, S(O)₂R₅, or (CR₁₀R₂₀)_{v′}OR₁₃;
R₃ is independently selected at each occurrence from hydrogen, halogen, C₁₋₄alkyl, or halosubstituted C₁₋₁₄alkyl;
R₄ and R₁₄ are each independently selected at each occurrence from hydrogen or C₁₋₄ alkyl, or R₄ and R₁₄ together with the nitrogen to which they are attached form a heterocyclic ring of 5 to 7 members, which ring optionally contains an additional heteroatom selected from NR₉;
R₅ is independently selected at each occurrence from hydrogen, C₁₋₄ alkyl, C₂₋₄ alkenyl, C₂₋₄ alkynyl or NR₄R₁₄, excluding the moieties SR₅ being SNR₄R₁₄, S(O)₂R₅ being SO₂H and S(O)R₅ being SOH;
R₉ and R_{9′} are independently selected at each occurrence from hydrogen, or C₁₋₄ alkyl;
R₁₂ is independently selected at each occurrence from hydrogen, C₁₋₄ alkyl, halo-substitutedC₁₋₄ alkyl, C₂₋₄ alkenyl, C₂₋₄ alkynyl, C₃₋₇cycloalkyl, C₃₋₇cycloalkylC₁₋₄ alkyl, C₅₋₇cycloalkenyl, C₅₋₇cycloalkenylC₁₋₄alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R₁₃ is independently selected at each occurrence from hydrogen, C₁₋₄ alkyl, halo-substituted C₁₋₄ alkyl, C₂₋₄ alkenyl, C₂₋₄ alkynyl, C₃₋₇ cycloalkyl, C₃₋₇cycloalkyl C₁₋₄ alkyl, C₅₋₇ cycloalkenyl, C₅₋₇cycloalkenyl C₁₋₄ alkyl, aryl, arylC₁₋₄ alkyl, heteroaryl, heteroarylC₁₋₄ alkyl, heterocyclyl, or a heterocyclylC₁₋₄ alkyl moiety, and wherein each of these moieties, excluding hydrogen, may be optionally substituted;
R_{d} and R_{d′} are each independently selected at each occurrence from hydrogen, C₁₋₄ alkyl, C₃₋₆cycloalkyl, C₃₋₆cycloalkyl C₁₋₄alkyl moiety, and wherein each of these moieties, excluding hydrogen, may be optionally substituted; or R_{d} and R_{d′} together with the nitrogen which they are attached form an optionally substituted heterocyclic ring of 5 to 6 members, which ring optionally contains an additional heteroatom selected from oxygen, sulfur or NR_{9′};
R_{b} is hydrogen, C₁₋₁₀ alkyl, C₃₋₇ cycloalkyl, C₃₋₇ cycloalkyl C₁₋₁₀ alkyl, aryl, arylC₁₋₁₀alkyl, heteroaryl, heteroarylC₁₋₁₀ alkyl, heterocyclic, or heterocyclylC₁₋₁₀ alkyl moiety, which moieties, excluding hydrogen, may all be optionally substituted;
R_{g} is C₁₋₁₀ alkyl, or aryl;
m is 0 or an integer having a value of 1, or 2;
s is an integer having a value of 1, 2, 3 or 4; and
t is an integer having a value of 1, 2, 3 or 4.
v is 0 or an integer having a value of 1 or 2;
v′ is independently selected at each occurrence from 0 or an integer having a value of 1 or 2;
Z is independently selected from oxygen or sulfur;
R₁₀ and R₂₀ are independently selected at each occurrence from hydrogen or C₁₋₄ alkyl; and
R_{10′} is independently selected at each occurrence from hydrogen or C₁₋₄alkyl;
which comprises decarboxylating a compound of Formula (II) according to claim 30 with a thioacid derivative to yield a compound of Formula (III).

50. The process according to claim 49 wherein the thioacids derivative is thioacetic acid, thiobenzoic acid, thioproprionic acid, sodium thioacetate, potassium thioacetate, lithium thioacetate, cesium thioacetate, or magnesium thioacetate.

51. The process according to claim 50 wherein the thioacids derivative is thioacetic acid.

52. The process according to claim 49 which further comprises an organic solvent, optionally in combination with water.

53. The process according to claim 52 wherein the solvent is selected from THF, toluene, DMF, n-methylpyrrolidine, methylene chloride, ethyl acetate, 2-methyl-THF, dioxane, DIPEA, pyridine, or acetonitrile.

54. The process according to claim 49 wherein the reaction is conducted at about 20 to about 50° C.

55. A process for producing a compound of Formula (II) according to claim 34 which comprises reacting a compound of Formula (IV) 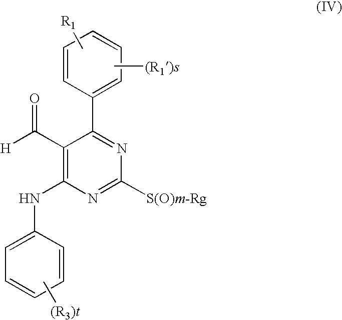
wherein R₁, R_{1′}, R₃, R_{g}, m, s and t are as described above for Formula (II), with a condensation agent selected from meldrums acid or malonic acid, in an organic solvent with a base to yield a compound of Formula (II).

56. The process according to claim 55 wherein the condensation agent is meldrum's acid.

57. The process according to claim 55 wherein the base is cesium hydroxide, cesium carbonate, sodium acetate, 2,4,6-collidine, DIPEA, DBN, dihexylamine, diethylamine, 2,6-dimethylpiperidine, di-secbutylamine, isopropylamine, isoquinoline, 2,6,-lutidine, N-methylpiperidine, pyridine, pyrrolidine, or triethylamine.

58. (canceled)

59. The process according to claim 55 wherein the organic solvent is selected from THF, toluene, DMF, n-methylpyrrolidine, methylene chloride, ethyl acetate, 2-methyl-THF, dioxane, or acetonitrile.

60-73. (canceled)

74. The process according to claim 55 wherein the compound of Formula (II) is decarboxylated in situ to yield a compound of Formula (III).

75. The process according to claim 55 wherein the compound of Formula (IV) is
4-(4-fluoro-2-methylphenyl)-2-(methylthio)-7-oxo-8-(2,6-difluorophenyl)-7,8-dihydropyrido[2,3-d]pyrimidine-6-carboxylic acid;
4-(2,4-difluorophenyl)-2-(methylthio)-7-oxo-8-(2,4,6-trifluorophenyl)-7,8-dihydropyrido[2,3-d]pyrimidine-6-carboxylic acid; or
8-(2,6-difluorophenyl)-4-(2-methyl-5-(methoxycarbonyl)phenyl)-2-(methylthio)-7-oxo-7,8-dihydropyrido[2,3-d]pyrimidine-6-carboxylic acid.

76. The process according to claim 74 wherein the compound of Formula (III) is
8-(2,6-Difluorophenyl)-4-(4-fluoro-2-methylphenyl)-2-(methylthio)pyrido[2,3-D]pyrimidin-7(8H)-one; or
3-[8-(2,6-difluorophenyl)-2-(methylthio)-7-oxo-7,8-dihydropyrido[2,3-d]pyrimidin-4-yl]-4-methyl benzoic methyl ester; or
4-(2,4-Difluorophenyl)-8-(2,4,6-trifluorophenyl)-2-(methylthio)pyrido[2,3-D]pyrimidin-7(8H)-one.

77-78. (canceled)

79. A polymorphic form, Form 1, of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate substantially as shown in at least one of the X-ray diffraction pattern of FIG. 5, the differential scanning calorimetry thermogram of FIG. 9, and the infrared spectrum of FIG. 13 (a) and/or 13(b).

80. A polymorphic form, Form 1, of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate wherein said polymorphic form is characterized by an x-ray diffraction pattern comprising peaks expressed in terms of 2 theta angles, wherein
i) said x-ray diffraction pattern comprises a peak at 8.2+/−0.1°; or
ii) said x-ray diffraction pattern comprises peaks at 7.5 and 8.2+/−0.1°; or
iii) said x-ray diffraction pattern comprises peaks at 7.5, 8.2, and 9.9+/−0.1°; or
iv) said x-ray diffraction pattern comprises peaks at 7.5, 8.2, 9.9, and 13.0+/−0.1°; or
v) said x-ray diffraction pattern comprises peaks at 7.5, 8.2, 9.9, 13.0, and 16.3+/−0.1°; or
vi) said x-ray diffraction pattern comprises peaks at 7.5, 8.2, 9.9, 13.0, 16.3, 19.8, and 21.1+/−0.1°; or
vii) said x-ray diffraction pattern comprises peaks at 7.5, 8.2, 9.9, 13.0, 16.3, 19.8, 21.1 and 21.8+/−0.1°.

81. A polymorphic form, Form 1 of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate having a powder X-ray diffraction pattern comprising a characteristic peak, in terms of 2θ at about 7.5+/−0.1° and 8.2+/−0.1° and at least 3 additional characteristic peaks in terms of 2θ, selected from 9.9+/−0.1°, 13.0+/−0.1°, 16.3+/−0.1°, 19.8+/−0.1°, 21.1+/−0.1° and 21.8+/−0.1°.

82. A polymorphic form, Form 1 of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate wherein said polymorphic form is characterized by an infrared spectrum comprising one or more characteristic peaks selected from about 3442, 3219, 3072, 2935, 1697, 1654, 1619, 1558, 1501, 1479, 1454, 1382, 1360, 1341, 1314, 1282, 1247, 1150, 1119, 1107, 1076, 1062, 1030, 1011, 1005, 983, 947, 913, 876, 838, 820, 798 and 709 cm⁻¹, and having a melt onset as calculated by DSC of about 230° C.

83. The polymorphic form, Form 1 according to claim 79 which is of substantially pure crystalline form.

84. A composition comprising a polymorphic form, Form 1 according to claim 79 wherein at least 30% by weight of total 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate in said composition is present as said polymorphic form.

85. The composition according to claim 84 wherein at least 50% by weight of polymorphic form, Form 1 is present.

86. The composition according to claim 84 wherein at least 70% by weight of polymorphic form, Form 1 is present.

87. The composition according to claim 84 wherein at least 90% by weight of polymorph Form 1 is present.

88. A pharmaceutical composition comprising a polymorphic form, Form 1 of 8-(2,6-difluorophenyl)-4-(4-fluoro-2-methylphenyl)-2-{[2-hydroxy-1-(hydroxymethyl)-ethyl]amino}pyrido[2,3-d]pyrimidin-7(8H)-one, tosylate, according to claim 79 and a pharmaceutically acceptable excipient or carrier.

89. A process for the preparation of a polymorphic form, Form 1 according to claim 79 comprising
a) dissolving 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate in a solvent which is methylene chloride and a co-solvent and warming if necessary to give a solution;
b) cooling the solution of step (a), optionally in an ice bath or optionally seed the solution with 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one tosylate polymorphic form, Form 1 to yield crystalline polymorphic form, Form 1.

90. The process according to claim 89 wherein the Form 1 produced in pure or substantially pure.

91. A process for the preparation of a polymorph according to claim 79 comprising suspending the tosylate in a solvent which is chloroform or a chloroform so-solvent mixture and then cooled for formation of polymorphic form, Form 1.

92. The process according to claim 91 wherein the co-solvent is methanol or ethanol.

93. The process according to claim 91 wherein the solvent is chloroform.

94.-99. (canceled)

100. A polymorphic form, Form 4, of 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate substantially as shown in the X-ray diffraction pattern of FIG. 8, or differential scanning calorimetry thermogram of FIG. 12, or the infrared spectrum of FIG. 16(a) and/or 16(b).

101.-116. (canceled)

117. The compound 8-(2,6-difluorophenyl)-4-(4-fluoro-2-methylphenyl)-2-{[2-hydroxy-1-(hydroxymethyl)ethyl]amino}pyrido[2,3-d]pyrimidin-7(8H)-one, tosylate in amorphous form.

118. A pharmaceutical composition comprising the amorphous compound according to claim 117 and a pharmaceutically acceptable carrier or diluent.
